SEQUENCE LISTING (112 sequences)

SEQ ID NO: 1 - DNA, 363 bp, Artificial Sequence
  Description of Artificial Sequence Synthetic polynucleotide
caggttcaactgcagcagtctggggctgagctggtgaggcctggggcttcagtgacgctgtcctgcaaggcttcgggctacacatttactgactatgaaatgcactgggtgaagcagacacctgtgcatggcctggaatggattggagctattgatcctgaaactggtggttctgcctacaatcagaagttcaaggccaaggccatactgactgcagacagatcctccagcacagcctacatggagctccgcagcctgacatctgaggactctgccgtctattactgtacaagaactcgttactacggaagtagtcctgctatggactactggggtcaaggaacctcagtcaccgtctcctca

SEQ ID NO: 2 - DNA, 321 bp, Artificial Sequence
  Description of Artificial Sequence Synthetic polynucleotide
gatatccagatgacacagactacatcctccctgtctgcctctctgggagacagagtcaccatcagttgcagtgcaagtcagggcattggcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccgtacacgttcggaggggggaccaagctggaaataaaa

SEQ ID NO: 3 - protein, 121 aa, Artificial Sequence
  Description of Artificial Sequence Synthetic polypeptide
QVQLQQSGAELVRPGASVTLSCKASGYTFTDYEMHWVKQTPVHGLEWIGAIDPETGGSAYNQKFKAKAILTADRSSSTAYMELRSLTSEDSAVYYCTRTRYYGSSPAMDYWGQGTSVTVSS

SEQ ID NO: 4 - protein, 107 aa, Artificial Sequence
  Description of Artificial Sequence Synthetic polypeptide
DIQMTQTTSSLSASLGDRVTISCSASQGIGNYLNWYQQKPDGTVKLLIYYTSSLHSGVPSRFSGSGSGTDYSLTISNLEPEDIATYYCQQYSKLPYTFGGGTKLEIK

SEQ ID NO: 5 - protein, 10 aa, Artificial Sequence
  Description of Artificial Sequence Synthetic peptide
GYTFTDYEMH

SEQ ID NO: 6 - protein, 17 aa, Artificial Sequence
  Description of Artificial Sequence Synthetic peptide
AIDPETGGSAYNQKFKA

SEQ ID NO: 7 - protein, 12 aa, Artificial Sequence
  Description of Artificial Sequence Synthetic peptide
TRYYGSSPAMDY

SEQ ID NO: 8 - protein, 11 aa, Artificial Sequence
  Description of Artificial Sequence Synthetic peptide
SASQGIGNYLN

SEQ ID NO: 9 - protein, 7 aa, Artificial Sequence
  Description of Artificial Sequence Synthetic peptide
YTSSLHS

SEQ ID NO: 10 - protein, 9 aa, Artificial Sequence
  Description of Artificial Sequence Synthetic peptide
QQYSKLPYT

SEQ ID NO: 11 - DNA, 639 bp, Artificial Sequence
  Description of Artificial Sequence Synthetic polynucleotide
atggaatggagctgggtctttctcttcctcctgtcagtaattgcaggtgtccaatcccaggttcaactgcagcagtctggggctgagctggtgaggcctggggcttcagtgacgctgtcctgcaaggcttcgggctacacatttactgactatgaaatgcactgggtgaagcagacacctgtgcatggcctggaatggattggagctattgatcctgaaactggtggttctgcctacaatcagaagttcaaggccaaggccatactgactgcagacagatcctccagcacagcctacatggagctccgcagcctgacatctgaggactctgccgtctattactgtacaagaactcgttactacggaagtagtcctgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagc

SEQ ID NO: 12 - DNA, 606 bp, Artificial Sequence
  Description of Artificial Sequence Synthetic polynucleotide
atgtcctctgctcagttccttggtctcctgttgctctgttttcaaggtaccagatgtgatatccagatgacacagactacatcctccctgtctgcctctctgggagacagagtcaccatcagttgcagtgcaagtcagggcattggcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaag

SEQ ID NO: 13 - protein, 213 aa, Artificial Sequence
  Description of Artificial Sequence Synthetic polypeptide
MEWSWVFLFLLSVIAGVQSQVQLQQSGAELVRPGASVTLSCKASGYTFTDYEMHWVKQTPVHGLEWIGAIDPETGGSAYNQKFKAKAILTADRSSSTAYMELRSLTSEDSAVYYCTRTRYYGSSPAMDYWGQGTSVTVSSAKTTPPSVYPLAPGSAAQTNSMVTLGCLVKGYFPEPVTVTWNSGSLSSGVHTFPAVLQSDLYTLSSSVTVPSS

SEQ ID NO: 14 - protein, 202 aa, Artificial Sequence
  Description of Artificial Sequence Synthetic polypeptide
MSSAQFLGLLLLCFQGTRCDIQMTQTTSSLSASLGDRVTISCSASQGIGNYLNWYQQKPDGTVKLLIYYTSSLHSGVPSRFSGSGSGTDYSLTISNLEPEDIATYYCQQYSKLPYTFGGGTKLEIKRADAAPTVSIFPPSSEQLTSGGASVVCFLNNFYPKDINVKWKIDGSERQNGVLNSWTDQDSKDSTYSMSSTLTLTK

SEQ ID NO: 15 - protein, 8 aa, Artificial Sequence
  Description of Artificial Sequence Synthetic peptide
GYTFTDYE

SEQ ID NO: 16 - protein, 8 aa, Artificial Sequence
  Description of Artificial Sequence Synthetic peptide
IDPETGGS

SEQ ID NO: 17 - protein, 14 aa, Artificial Sequence
  Description of Artificial Sequence Synthetic peptide
TRTRYYGSSPAMDY

SEQ ID NO: 18 - protein, 6 aa, Artificial Sequence
  Description of Artificial Sequence Synthetic peptide
QGIGNY

SEQ ID NO: 19 - protein, 3 aa, Artificial Sequence
  Description of Artificial Sequence Synthetic peptide
YTS

SEQ ID NO: 20 - protein, 9 aa, Artificial Sequence
  Description of Artificial Sequence Synthetic peptide
QQYSKLPYT

SEQ ID NO: 21 - DNA, 636 bp, Artificial Sequence
  Description of Artificial Sequence Synthetic polynucleotide
atggaatggagctgggtctttctcttcctcctgtcagtaattgcaggtgtccaatcccaggttcaactgcagcagtctggggctgagctggtgaggcctggggcttcagtgacgctgtcctgcaaggcttcgggctacacatttactgactatgaaattcactggatgaagcagacacctgtgcatggcctggaatggattggaactattgatcctgaaactggtggtactgcctacaatcagaagttcaagggcaaggccatactgactgcagacaaatcctccagcacagcctacatggagctccgcagcctgacatctgaggactctgccgtctattactgtacaagatgttactacggtagtagttggtacttcgatgtctggggcacagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagc

SEQ ID NO: 22 - DNA, 606 bp, Artificial Sequence
  Description of Artificial Sequence Synthetic polynucleotide
atgtcctctgctcagttccttggtctcctgttgctctgttttcaaggtaccagatgtgatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacttgaagatattgccacttactattgtcagcagtatagtaaggttcctcggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaag

SEQ ID NO: 23 - protein, 212 aa, Artificial Sequence
  Description of Artificial Sequence Synthetic polypeptide
MEWSWVFLFLLSVIAGVQSQVQLQQSGAELVRPGASVTLSCKASGYTFTDYEIHWMKQTPVHGLEWIGTIDPETGGTAYNQKFKGKAILTADKSSSTAYMELRSLTSEDSAVYYCTRCYYGSSWYFDVWGTGTTVTVSSAKTTPPSVYPLAPGSAAQTNSMVTLGCLVKGYFPEPVTVTWNSGSLSSGVHTFPAVLQSDLYTLSSSVTVPSS

SEQ ID NO: 24 - protein, 202 aa, Artificial Sequence
  Description of Artificial Sequence Synthetic polypeptide
MSSAQFLGLLLLCFQGTRCDIQMTQTTSSLSASLGDRVTISCSASQGISNYLNWYQQKPDGTVKLLIYYTSSLHSGVPSRFSGSGSGTDYSLTISNLELEDIATYYCQQYSKVPRTFGGGTKLEIKRADAAPTVSIFPPSSEQLTSGGASVVCFLNNFYPKDINVKWKIDGSERQNGVLNSWTDQDSKDSTYSMSSTLTLTK

SEQ ID NO: 25 - protein, 8 aa, Artificial Sequence
  Description of Artificial Sequence Synthetic peptide
GYTFTDYE

SEQ ID NO: 26 - protein, 8 aa, Artificial Sequence
  Description of Artificial Sequence Synthetic peptide
IDPETGGT

SEQ ID NO: 27 - protein, 13 aa, Artificial Sequence
  Description of Artificial Sequence Synthetic peptide
TRCYYGSSWYFDV

SEQ ID NO: 28 - protein, 6 aa, Artificial Sequence
  Description of Artificial Sequence Synthetic peptide
QGITNY

SEQ ID NO: 29 - protein, 3 aa, Artificial Sequence
  Description of Artificial Sequence Synthetic peptide
YTS

SEQ ID NO: 30 - protein, 9 aa, Artificial Sequence
  Description of Artificial Sequence Synthetic peptide
QQYSKVPRT

SEQ ID NO: 31 - DNA, 30 bp, Artificial Sequence
  Description of Artificial Sequence Synthetic oligonucleotide
ggctacacatttactgactatgaaatgcac

SEQ ID NO: 32 - DNA, 51 bp, Artificial Sequence
  Description of Artificial Sequence Synthetic oligonucleotide
gctattgatcctgaaactggtggttctgcctacaatcagaagttcaaggcc

SEQ ID NO: 33 - DNA, 36 bp, Artificial Sequence
  Description of Artificial Sequence Synthetic oligonucleotide
actcgttactacggaagtagtcctgctatggactac

SEQ ID NO: 34 - DNA, 33 bp, Artificial Sequence
  Description of Artificial Sequence Synthetic oligonucleotide
agtgcaagtcagggcattggcaattatttaaac

SEQ ID NO: 35 - DNA, 21 bp, Artificial Sequence
  Description of Artificial Sequence Synthetic oligonucleotide
tacacatcaagtttacactca

SEQ ID NO: 36 - DNA, 27 bp, Artificial Sequence
  Description of Artificial Sequence Synthetic oligonucleotide
cagcagtatagtaagcttccgtacacg

SEQ ID NO: 37 - DNA, 27 bp, Artificial Sequence
  Description of Artificial Sequence Synthetic oligonucleotide
ggctacacatttactgactatgaaatg

SEQ ID NO: 38 - DNA, 24 bp, Artificial Sequence
  Description of Artificial Sequence Synthetic oligonucleotide
attgatcctgaaactggtggttct

SEQ ID NO: 39 - DNA, 42 bp, Artificial Sequence
  Description of Artificial Sequence Synthetic oligonucleotide
acaagaactcgttactacggaagtagtcctgctatggactac

SEQ ID NO: 40 - DNA, 18 bp, Artificial Sequence
  Description of Artificial Sequence Synthetic oligonucleotide
cagggcattggcaattat

SEQ ID NO: 41 - DNA, 9 bp, Artificial Sequence
  Description of Artificial Sequence Synthetic oligonucleotide
tacacatca

SEQ ID NO: 42 - DNA, 27 bp, Artificial Sequence
  Description of Artificial Sequence Synthetic oligonucleotide
cagcagtatagtaagcttccgtacacg

SEQ ID NO: 43 - DNA, 24 bp, Artificial Sequence
  Description of Artificial Sequence Synthetic oligonucleotide
ggctacacatttactgactatgaa

SEQ ID NO: 44 - DNA, 24 bp, Artificial Sequence
  Description of Artificial Sequence Synthetic oligonucleotide
attgatcctgaaactggtggtact

SEQ ID NO: 45 - DNA, 39 bp, Artificial Sequence
  Description of Artificial Sequence Synthetic oligonucleotide
acaagatgttactacggtagtagttggtacttcgatgtc

SEQ ID NO: 46 - DNA, 18 bp, Artificial Sequence
  Description of Artificial Sequence Synthetic oligonucleotide
cagggcattagcaattat

SEQ ID NO: 47 - DNA, 9 bp, Artificial Sequence
  Description of Artificial Sequence Synthetic oligonucleotide
tacacatca

SEQ ID NO: 48 - DNA, 27 bp, Artificial Sequence
  Description of Artificial Sequence Synthetic oligonucleotide
cagcagtatagtaaggttcctcggacg

SEQ ID NO: 49 - protein, 7 aa, Artificial Sequence
  Description of Artificial Sequence Synthetic peptide
IDPETGG

SEQ ID NO: 50 - protein, 5 aa, Artificial Sequence
  Description of Artificial Sequence Synthetic peptide
YYGSS

SEQ ID NO: 51 - protein, 6 aa, Artificial Sequence
  Description of Artificial Sequence Synthetic peptide
  MOD_RES (4)..(4): Any amino acid
QGIXNY

SEQ ID NO: 52 - protein, 9 aa, Artificial Sequence
  Description of Artificial Sequence Synthetic peptide
  MOD_RES (6)..(6): Any amino acid
  MOD_RES (8)..(8): Any amino acid
QQYSKXPXT

SEQ ID NO: 53 - protein, 19 aa, Homo sapiens
LTISGEDKILQNTAPKSRN

SEQ ID NO: 54 - protein, 8 aa, Homo sapiens
ILQNTAPK

SEQ ID NO: 55 - DNA, 4863 bp, Homo sapiens
atgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 56 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 57 - protein, 682 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVH

SEQ ID NO: 58 - protein, 10 aa, Artificial Sequence
  Description of Artificial Sequence Synthetic peptide
GGGGSGGGGS

SEQ ID NO: 59 - protein, 5 aa, Artificial Sequence
  Description of Artificial Sequence Synthetic peptide
GGGGS

SEQ ID NO: 60 - protein, 15 aa, Artificial Sequence
  Description of Artificial Sequence Synthetic peptide
GGGGSGGGGSGGGGS

SEQ ID NO: 61 - protein, 20 aa, Artificial Sequence
  Description of Artificial Sequence Synthetic peptide
GGGGSGGGGSGGGGSGGGGS

SEQ ID NO: 62 - protein, 25 aa, Artificial Sequence
  Description of Artificial Sequence Synthetic peptide
GGGGSGGGGSGGGGSGGGGSGGGGS

SEQ ID NO: 63 - protein, 30 aa, Artificial Sequence
  Description of Artificial Sequence Synthetic polypeptide
GGGGSGGGGSGGGGSGGGGSGGGGSGGGGS

SEQ ID NO: 64 - protein, 35 aa, Artificial Sequence
  Description of Artificial Sequence Synthetic polypeptide
GGGGSGGGGSGGGGSGGGGSGGGGSGGGGSGGGGS

SEQ ID NO: 65 - protein, 20 aa, Artificial Sequence
  Description of Artificial Sequence Synthetic peptide
  SITE (1)..(20): This sequence may encompass 1-10 “Gly Ser” repeating units
GSGSGSGSGSGSGSGSGSGS

SEQ ID NO: 66 - protein, 30 aa, Artificial Sequence
  Description of Artificial Sequence Synthetic polypeptide
  SITE (1)..(30): This sequence may encompass 1-10 “Gly Gly Ser” repeating units
GGSGGSGGSGGSGGSGGSGGSGGSGGSGGS

SEQ ID NO: 67 - protein, 40 aa, Artificial Sequence
  Description of Artificial Sequence Synthetic polypeptide
  SITE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 68 - protein, 40 aa, Artificial Sequence
  Description of Artificial Sequence Synthetic polypeptide
  SITE (1)..(40): This sequence may encompass 1-10 “Gly Gly Ser Gly” repeating units
GGSGGGSGGGSGGGSGGGSGGGSGGGSGGGSGGGSGGGSG

SEQ ID NO: 69 - protein, 50 aa, Artificial Sequence
  Description of Artificial Sequence Synthetic polypeptide
  SITE (1)..(50): This sequence may encompass 1-10 “Gly Gly Ser Gly Gly” repeating units
GGSGGGGSGGGGSGGGGSGGGGSGGGGSGGGGSGGGGSGGGGSGGGGSGG

SEQ ID NO: 70 - protein, 50 aa, Artificial Sequence
  Description of Artificial Sequence Synthetic polypeptide
  SITE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

SEQ ID NO: 71 - protein, 4 aa, Artificial Sequence
  Description of Artificial Sequence Synthetic peptide
CPSC

SEQ ID NO: 72 - protein, 4 aa, Artificial Sequence
  Description of Artificial Sequence Synthetic peptide
CPPC

SEQ ID NO: 73 - protein, 42 aa, Artificial Sequence
  Description of Artificial Sequence Synthetic polypeptide
ISGEDKILQNTAPKSRNLERNPNKELKPGENSPRQTPIFDPC

SEQ ID NO: 74 - protein, 8 aa, Artificial Sequence
  Description of Artificial Sequence Synthetic peptide
DYKDDDDK

SEQ ID NO: 75 - DNA, 21 bp, Artificial Sequence
  Description of Artificial Sequence Synthetic oligonucleotide
cattcagggagacgcccattt

SEQ ID NO: 76 - DNA, 21 bp, Artificial Sequence
  Description of Artificial Sequence Synthetic oligonucleotide
ccacaacatcacctattggat

SEQ ID NO: 77 - DNA, 21 bp, Artificial Sequence
  Description of Artificial Sequence Synthetic oligonucleotide
ccctacaaatcctttccgttt

SEQ ID NO: 78 - DNA, 21 bp, Artificial Sequence
  Description of Artificial Sequence Synthetic oligonucleotide
gcaggttctatctgtgagaaa

SEQ ID NO: 79 - DNA, 21 bp, Artificial Sequence
  Description of Artificial Sequence Synthetic oligonucleotide
cctggttacaactcatgaatt

SEQ ID NO: 80 - DNA, 21 bp, Artificial Sequence
  Description of Artificial Sequence Synthetic oligonucleotide
cccatgaagaacacgtgtaaa

SEQ ID NO: 81 - DNA, 21 bp, Artificial Sequence
  Description of Artificial Sequence Synthetic oligonucleotide
cctatgtcgatgctgaacaaa

SEQ ID NO: 82 - DNA, 21 bp, Artificial Sequence
  Description of Artificial Sequence Synthetic oligonucleotide
ccagcagcattcggtaagaaa

SEQ ID NO: 83 - DNA, 21 bp, Artificial Sequence
  Description of Artificial Sequence Synthetic oligonucleotide
gcagcgtcagaatcgtgttaa

SEQ ID NO: 84 - protein, 57 aa, Homo sapiens
SLDCYLTISGEDKILQNTAPKSRNLFERNPNKELKPGENSPRQTPIFDPTVHWLFTT

SEQ ID NO: 85 - DNA, 13 bp, Artificial Sequence
  Description of Artificial Sequence Synthetic oligonucleotide
agaaacgagagaa

SEQ ID NO: 86 - DNA, 19 bp, Homo sapiens
agaaacctgtttgagagaa

SEQ ID NO: 87 - protein, 6 aa, Homo sapiens
RNLFER

SEQ ID NO: 88 - protein, 60 aa, Rattus norvegicus
TVAFDNISISLDCYLTISGEEKMSLNAVPKSRNLFEKNPNKEPKPWANISGPTPIFDPTV

SEQ ID NO: 89 - protein, 60 aa, Mus musculus
TVGFDNISISLDCYLTISGEEKMSLNSVPKSRNLFEKNPNKESKSWANISGPTPIFDPTV

SEQ ID NO: 90 - protein, 60 aa, Dasypus novemcinctus
TVAFDNISVSLDCYLTISGEDTVPQNAAPKSRNLFDRNPNKEPKPWESTPRETPIFDPTV

SEQ ID NO: 91 - protein, 57 aa, Echinops telfairi
TVAFDNISISLDCYLTISGEDKNAAPKSRNLFERNPNKELTPWRNPPRETPIFDPTV

SEQ ID NO: 92 - protein, 60 aa, Trichechus manatus
TVAFDNISISLDCYLTISGEDRMPQNTAPKSRNLFERSPNKEMKPWGNTPRETPIFDPTV

SEQ ID NO: 93 - protein, 60 aa, Bos taurus
TVAFDNISVSLDCYLTISGEDKIPQNTAPKSRNLFERNLNNELKPWVNPSKETPIFDPTV

SEQ ID NO: 94 - protein, 60 aa, Condylura cristata
TVAFDNISISLDCYLTISGEDKMPQNEAPKSRNLFERNSSKEPKTGESTPSETPTFDPTV

SEQ ID NO: 95 - protein, 59 aa, Canis lupus familiaris
TVAFDNISISLDCYLTISGEEKMLQNTAPKSRNLFERNPNKDPRPWENTRETPVFDPTV

SEQ ID NO: 96 - protein, 60 aa, Homo sapiens
IVAFDNISISLDCYLTISGEDKILQNTAPKSRNLFERNPNKELKPGENSPRQTPIFDPTV

SEQ ID NO: 97 - protein, 60 aa, Microcebus murinus
TVAFDNISISLDCYLTISGEDKMSQNAAPKSRNLFDRNPNVELKPLENIPRQTPDFDPTV

SEQ ID NO: 98 - protein, 22 aa, Chelonia mydas
DIAMDNITFGVGCFPVGNVPVV

SEQ ID NO: 99 - protein, 52 aa, Ictalurus punctatus
DIALDDISLSATCFDTDLKALLLGRQHDLDSISQLDFLSPLPEPSASEVSQI

SEQ ID NO: 100 - protein, 51 aa, Zootermopsis nevadensis
YMSLDDFSMSPECFGFGVPANVSGDYVYNSTYCYQTDAADKDIHQDFINKT

SEQ ID NO: 101 - protein, 47 aa, Drosophila melanogaster
DVAVDDFSLSPECFGLNIPEDHLGGYNYWDVRQNLKSPTYKDFEYTN

SEQ ID NO: 102 - protein, 10 aa, Homo sapiens
PKSRNLFERN

SEQ ID NO: 103 - protein, 4 aa, Artificial Sequence
  Description of Artificial Sequence Synthetic peptide
PERN

SEQ ID NO: 104 - protein, 10 aa, Artificial Sequence
  Description of Artificial Sequence Synthetic peptide
PKSRNRDERN

SEQ ID NO: 105 - protein, 10 aa, Artificial Sequence
  Description of Artificial Sequence Synthetic peptide
PKSRKRDERN

SEQ ID NO: 106 - protein, 10 aa, Artificial Sequence
  Description of Artificial Sequence Synthetic peptide
PKSRNFYERN

SEQ ID NO: 107 - protein, 8 aa, Artificial Sequence
  Description of Artificial Sequence Synthetic peptide
PKSRNERN

SEQ ID NO: 108 - DNA, 27 bp, Artificial Sequence
  Description of Artificial Sequence Synthetic oligonucleotide
acccaaatcaagaaacctaaacaagga

SEQ ID NO: 109 - DNA, 32 bp, Homo sapiens
agaaacctgtttgagagaaacccaaacaagga

SEQ ID NO: 110 - DNA, 17 bp, Artificial Sequence
  Description of Artificial Sequence Synthetic oligonucleotide
agaaacctaaacaagga

SEQ ID NO: 111 - DNA, 17 bp, Artificial Sequence
  Description of Artificial Sequence Synthetic oligonucleotide
agaaacccaaacaagga

SEQ ID NO: 112 - protein, 9 aa, Artificial Sequence
  Description of Artificial Sequence Synthetic peptide
  MOD_RES (3)..(3): L or P
RNXFERNPN

(19) United States
(12) Patent Application Publication
(10) Pub. No.: US 2024/0262917 A1
(43) Pub. Date: Aug. 8, 2024

(54) ANTIBODIES AGAINST ALK AND METHODS OF USE THEREOF

(71) Applicant: Dana-Farber Cancer Institute, Inc., Boston, MA (US)

(72) Inventors: Rani E. GEORGE, Jamaica Plain, MA (US); Bandana SHARMA, Brookline, MA (US); Edward GREENFIELD, Stoughton, MA (US)

(73) Assignee: Dana-Farber Cancer Institute, Inc., Boston, MA (US)

(21) Appl. No.: 18/289,568

(22) PCT Filed: May 5, 2022
(86) PCT No.: PCT/US2022/027882

§ 371 Date: Nov. 5, 2023

Related U.S. Application Data
(60) Provisional application No. 63/185,112, filed on May 6, 2021.

(51) Int. Cl. C07K 16/28; A61K 39/00; A61K 47/68; A61K 51/10; A61P 35/04; C12N 5/0783; G01N 33/574

(52) U.S. Cl. CPC C07K 16/2863; A61K 39/4611; A61K 39/4613; A61K 39/4631; A61K 39/464403; A61K 47/6813; A61K 47/6849; A61K 51/103; A61P 35/04; C12N 5/0636; C12N 5/0646; G01N 33/57407; A61K 2039/505; A61K 2121/00; C07K 2317/21; C07K 2317/24; C07K 2317/31; C12N 2510/00; G01N 2333/912

(57) ABSTRACT

The present invention is directed to human monoclonal antibodies that bind to Anaplastic Lymphoma Kinase (ALK).

### CROSS-REFERENCE TO RELATED APPLICATIONS

This application is a National Stage application, filed under 35 U.S.C. 371, of International Application No. PCT/US2022/027882, filed on May 5, 2022, which claims benefit of U.S. Provisional Application No. 63/185,112, filed May 6, 2021, the entire contents of the latter being hereby incorporated herein by reference.

### SEQUENCE LISTING

The instant application contains a Sequence Listing which has been submitted electronically in ASCII format and is hereby incorporated by reference in its entirety. Said ASCII copy, created on Jun. 15, 2022, is named 5031461-000099-WO1_SL.txt and is 71,783 bytes in size.

### FIELD

Antibodies against ALK (also known as Anaplastic Lymphoma Kinase), and methods of use thereof, are provided.

### BACKGROUND

The full-length anaplastic lymphoma kinase (ALK) tyrosine receptor kinase consists of an extracellular (ECD), a transmembrane and an intracellular domain. The ECD, which contains binding sites for the ALK ligands FAM100A and FAM100B, consists of an N-terminal signal peptide, two meprin-A5-protein receptor protein tyrosine phosphatase Mu (MAM1 and MAM2) domains separate by a low-density lipoprotein class A motif (LDLa), and a glycine-rich region. The intracellular domain of ALK includes a catalytic protein tyrosine kinase (PTK) domain.

The anaplastic lymphoma kinase (ALK) tyrosine kinase receptor is aberrantly expressed in several cancers through translocation or point mutation, leading to the use of ALK inhibitors in the clinic. In neuroblastoma, ALK mutations occur as single nucleotide alterations or amplification of the full length receptor and are seen in approximately 10% of tumors. By contrast, the wild-type receptor, whose expression is limited to the nervous system during development, is aberrantly expressed in the vast majority of neuroblastomas, and is associated with a poor patient outcome.

### SUMMARY

Aspects of the invention are directed to isolated monoclonal antibodies or fragments thereof that specifically bind to Anaplastic Lymphoma Kinase (ALK).

In embodiments, the monoclonal antibody comprises a heavy chain, light chain or a combination thereof.

In embodiments, ALK is human ALK.

In embodiments, the antibodies were identified in ALK−/− mice.

In embodiments, the antibody is fully human or humanized.

In embodiments, the antibody is monospecific, bispecific, or multispecific.

In embodiments, the antibody is an IgG.

In embodiments, the antibody comprises 8G7, 5H3 and 7F7. In other embodiments, the antibody competes with the binding of 8G7, 5H3 and 7F7.

In embodiments, the antibody comprises a heavy chain with three CDRs comprising the amino acid sequence GYTFTDYE (SEQ ID NO: 15), IDPETGG (SEW ID NO: 49), and YYGSS (SEQ ID NO: 50), and a light chain with three CDRs comprising QGIXNY (SEQ ID NO: 51), YTS (SEQ ID NO: 19), QQYSKXPXT (SEQ ID NO: 52).

In embodiments, the antibody comprises a heavy chain with three CDRs comprising the amino acid sequences GYTFTDYEMH (SEQ ID NO:5), AIDPETGGSAYNQKFKA (SEQ ID NO:6), and TRYYGSSPAMDY (SEQ ID NO:7) respectively, and/or a light chain with three CDRs comprising the amino acid sequences SASQGIGNYLN (SEQ ID NO:8), YTSSLHS (SEQ ID NO:9), and QQYSKLPYT (SEQ ID NO:10) respectively; a heavy chain with three CDRs comprising the amino acid sequences GYTFTDYE (SEQ ID NO:15), IDPETGGS (SEQ ID NO:16), and TRTRYYGSSPAMDY (SEQ ID NO:17) respectively, and/or a light chain with three CDRs comprising the amino acid sequences QGIGNY (SEQ ID NO:18), YTS (SEQ ID NO:19), and QQYSKLPYT (SEQ ID NO:20) respectively; and heavy chain with three CDRs comprising the amino acid sequences GYTFTDYE (SEQ ID NO:25), IDPETGGT (SEQ ID NO:26), and TRCYYGSSWYFDV (SEQ ID NO:27) respectively, and/or a light chain with three CDRs comprising the amino acid sequences QGITNY (SEQ ID NO:28), YTS (SEQ ID NO:29), and QQYSKVPRT (SEQ ID NO:30) respectively.

In embodiments, the antibody comprises a VH amino acid sequence having SEQ ID NO: 03, SEQ ID NO: 13, or SEQ ID NO: 23, or a sequence at least 90% identical thereto.

In embodiments, the antibody comprises a VL amino acid sequence having SEQ ID NO: 04, SEQ ID NO: 14, or SEQ ID NO: 24, or a sequence at least 90% identical thereto.

In embodiments, the antibody comprises a VH according to the amino acid sequence of SEQ ID NO: 03 or at least 90% identical thereto, and a VL according to the amino acid sequence of SEQ ID NO: 04 or at least 90% identical thereto; a VH according to the amino acid sequence of SEQ ID NO: 13 or at least 90% identical thereto, and a VL according to the amino acid sequence of SEQ ID NO: 14 or at least 90% identical thereto; or a VH according to the amino acid sequence of SEQ ID NO: 23 or at least 90% identical thereto, and a VL according to the amino acid sequence of SEQ ID NO: 24 or at least 90% identical thereto.

In embodiments, the antibody comprises a VH encoded by a nucleic acid according to SEQ ID NO: 01, SEQ ID NO: 11, or SEQ ID NO: 21, or a sequence at least 90% identical thereto.

In embodiments, the antibody comprises a VL encoded by a nucleic acid according to SEQ ID NO: 02, SEQ ID NO: 12, SEQ ID NO: 22, or a sequence at least 90% identical thereto.

In embodiments, the antibody is encoded by a VH encoded by a nucleic acid according to SEQ ID NO: 01 or at least 90% identical thereto, and a VL encoded by a nucleic acid according to SEQ ID NO: 2 or at least 90% identical thereto; a VH encoded by a nucleic acid according to SEQ ID NO: 11 or at least 90% identical thereto, and a VL encoded by a nucleic acid according to SEQ ID NO: 12 or at least 90% identical thereto; or a VH encoded by a nucleic acid according to SEQ ID NO: 21 or at least 90% identical thereto, and a VL encoded by a nucleic acid according to SEQ ID NO: 22 or at least 90% identical thereto.

In embodiments, the antibody is linked to a therapeutic agent. For example, the therapeutic agent is a toxin, a radiolabel, a siRNA, a small molecule, or a cytokine.

In embodiments, the antibody is a single chain fragment.

Aspects of the invention are also drawn to a nucleic acid encoding an antibody as described herein. For example, an embodiment of the invention can comprise a vector comprising a nucleic acid described herein.

Embodiments of the invention can also comprise a cell comprising a nucleic acid or vector as described herein. For example, the cell can produce an antibody as described herein.

Still further, aspects of the invention are drawn to a kit, for example, a kit comprising an antibody as described herein; a syringe, needle, or applicator for administration of the at least one antibody to a subject; and instructions for use.

Aspects of the invention are also drawn towards an isolated scFv antibody that specifically binds to Anaplastic Lymphoma Kinase (ALK), comprising a heavy chain, light chain or a combination thereof. For example, the scFv antibody comprises a heavy chain with three CDRs comprising the amino acid sequence GYTFTDYE (SEQ ID NO: 15), IDPETGG (SEQ ID NO: 49), and YYGSS (SEQ ID NO: 50), and a light chain with three CDRs comprising QGIXNY (SEQ ID NO: 51), YTS (SEQ ID NO: 19), QQYSKXPXT (SEQ ID NO: 52).

In embodiments, the scFv antibody comprises: a heavy chain with three CDRs comprising the amino acid sequences GYTFTDYEMH (SEQ ID NO:5), AIDPETGGSAYNQKFKA (SEQ ID NO:6), and TRYYGSSPAMDY (SEQ ID NO:7) respectively, and/or a light chain with three CDRs comprising the amino acid sequences SASQGIGNYLN (SEQ ID NO:8), YTSSLHS (SEQ ID NO:9), and QQYSKLPYT (SEQ ID NO:10) respectively; a heavy chain with three CDRs comprising the amino acid sequences GYTFTDYE (SEQ ID NO:15), IDPETGGS (SEQ ID NO:16), and TRTRYYGSSPAMDY (SEQ ID NO:17) respectively, and/or a light chain with three CDRs comprising the amino acid sequences QGIGNY (SEQ ID NO:18), YTS (SEQ ID NO:19), and QQYSKLPYT (SEQ ID NO:20) respectively; and heavy chain with three CDRs comprising the amino acid sequences GYTFTDYE (SEQ ID NO:25), IDPETGGT (SEQ ID NO:26), and TRCYYGSSWYFDV (SEQ ID NO:27) respectively, and/or a light chain with three CDRs comprising the amino acid sequences QGITNY (SEQ ID NO:28), YTS (SEQ ID NO:29), and QQYSKVPRT (SEQ ID NO:30) respectively.

Embodiments of the invention comprise an scFv antibody comprising a VH amino acid sequence having SEQ ID NO: 03, SEQ ID NO: 13, or SEQ ID NO: 23, or a sequence at least 90% identical thereto.

Embodiments of the invention comprise an scFv antibody comprising a VL amino acid sequence having SEQ ID NO: 04, SEQ ID NO: 14, or SEQ ID NO: 24, or a sequence at least 90% identical thereto.

In embodiments, the scFv antibody comprises a VH according to the amino acid sequence of SEQ ID NO: 03 or at least 90% identical thereto, and a VL according to the amino acid sequence of SEQ ID NO: 04 or at least 90% identical thereto; a VH according to the amino acid sequence of SEQ ID NO: 13 or at least 90% identical thereto, and a VL according to the amino acid sequence of SEQ ID NO: 14 or at least 90% identical thereto; or a VH according to the amino acid sequence of SEQ ID NO: 23 or at least 90% identical thereto, and a VL according to the amino acid sequence of SEQ ID NO: 24 or at least 90% identical thereto.

In embodiments, the scFv antibody comprises a VH encoded by a nucleic acid according to SEQ ID NO: 01, SEQ ID NO: 11, or SEQ ID NO: 21, or a sequence at least 90% identical thereto.

In embodiments, the scFv antibody comprises a VL encoded by a nucleic acid according to SEQ ID NO: 02, SEQ ID NO: 12, SEQ ID NO: 22, or a sequence at least 90% identical thereto.

In embodiments, the scFv antibody comprises a VH encoded by a nucleic acid according to SEQ ID NO: 01 or at least 90% identical thereto, and a VL encoded by a nucleic acid according to SEQ ID NO: 2 or at least 90% identical thereto; a VH encoded by a nucleic acid according to SEQ ID NO: 11 or at least 90% identical thereto, and a VL encoded by a nucleic acid according to SEQ ID NO: 12 or at least 90% identical thereto; or a VH encoded by a nucleic acid according to SEQ ID NO: 21 or at least 90% identical thereto, and a VL encoded by a nucleic acid according to SEQ ID NO: 22 or at least 90% identical thereto.

Embodiments also comprise a nucleic acid encoding the scFv antibody as described herein.

Embodiments further comprise a vector comprising the nucleic acid encoding the scFv antibody as described herein

Embodiments also comprise a cell comprising a nucleic acid as described herein, or a vector as described herein.

Embodiments further comprise a cell producing the scFv antibody as described herein.

Further aspects of the invention are also drawn to a kit comprising the scFv antibody as described herein; a syringe, needle, or applicator for administration of the at least one antibody to a subject; and instructions for use.

In embodiments, the scFv antibody is linked to a therapeutic agent. For example, the therapeutic agent is a toxin, a radiolabel, a siRNA, a small molecule, or a cytokine.

Aspects of the invention are further drawn to a pharmaceutical composition. For example, the pharmaceutical composition can comprise one or more antibody compositions as described herein and a pharmaceutically acceptable carrier or excipient.

In embodiments, the pharmaceutical composition can comprise at least one additional therapeutic agent. For example, the therapeutic agent can be a toxin, a radiolabel, a siRNA, a small molecule, or a cytokine.

Aspects of the invention are also drawn to an engineered cell comprising a chimeric antigen receptor. For example, the chimeric antigen receptor comprises an extracellular ligand binding domain that is specific for an antigen on the surface of a cancer cell, wherein the antigen comprises anaplastic lymphoma kinase (ALK). In embodiments, the extracellular ligand binding domain comprises an antibody or fragment thereof. In embodiments, the antibody comprises a heavy chain with three CDRs comprising the amino acid sequence GYTFTDYE (SEQ ID NO: 15), IDPETGG (SEQ ID NO: 49), and YYGSS (SEQ ID NO: 50), and a light chain with three CDRs comprising QGIXNY (SEQ ID NO: 51), YTS (SEQ ID NO: 19), QQYSKXPXT (SEQ ID NO: 52).

For example, embodiments of the engineered cell comprises a heavy chain with three CDRs comprising the amino acid sequences GYTFTDYEMH (SEQ ID NO:5), AIDPETGGSAYNQKFKA (SEQ ID NO:6), and TRYYGSSPAMDY (SEQ ID NO:7) respectively, and/or a light chain with three CDRs comprising the amino acid sequences SASQGIGNYLN (SEQ ID NO:8), YTSSLHS (SEQ ID NO:9), and QQYSKLPYT (SEQ ID NO:10) respectively; a heavy chain with three CDRs comprising the amino acid sequences GYTFTDYE (SEQ ID NO:15), IDPETGGS (SEQ ID NO:16), and TRTRYYGSSPAMDY (SEQ ID NO: 17) respectively, and/or a light chain with three CDRs comprising the amino acid sequences QGIGNY (SEQ ID NO:18), YTS (SEQ ID NO:19), and QQYSKLPYT (SEQ ID NO:20) respectively; and a heavy chain with three CDRs comprising the amino acid sequences GYTFTDYE (SEQ ID NO:25), IDPETGGT (SEQ ID NO:26), and TRCYYGSSWYFDV (SEQ ID NO:27) respectively, and/or a light chain with three CDRs comprising the amino acid sequences QGITNY (SEQ ID NO:28), YTS (SEQ ID NO:29), and QQYSKVPRT (SEQ ID NO:30) respectively.

In embodiments, the antibody comprises a VH amino acid sequence having SEQ ID NO: 03, SEQ ID NO: 13, or SEQ ID NO: 23, or a sequence at least 90% identical thereto.

In embodiments, the antibody comprises a VL amino acid sequence having SEQ ID NO: 04, SEQ ID NO: 14, or SEQ ID NO: 24, or a sequence at least 90% identical thereto.

In embodiments, the antibody comprises a VH according to the amino acid sequence of SEQ ID NO: 03 or at least 90% identical thereto, and a VL according to the amino acid sequence of SEQ ID NO: 04 or at least 90% identical thereto; a VH according to the amino acid sequence of SEQ ID NO: 13 or at least 90% identical thereto, and a VL according to the amino acid sequence of SEQ ID NO: 14 or at least 90% identical thereto; or a VH according to the amino acid sequence of SEQ ID NO: 23 or at least 90% identical thereto, and a VL according to the amino acid sequence of SEQ ID NO: 24 or at least 90% identical thereto.

In embodiments, the cell comprises a T cell, an NK cell, or an NKT cell. For example, the T cell is CD4+, CD8+, CD3+ pan T cells, or any combination thereof.

Aspects of the invention are also drawn to a kit comprising an engineered cell described herein, a syringe, needle, or applicator for administration of the engineered cell to a subject; and instructions for use.

Embodiments, for example, can comprise a pharmaceutical composition comprising an engineered cell described herein and a pharmaceutically acceptable carrier or excipient. In embodiments, the pharmaceutical composition can comprise at least one additional therapeutic agent. For example, the therapeutic agent is a toxin, a radiolabel, a siRNA, a small molecule, or a cytokine.

Aspects of the invention are drawn to methods of detecting the presence of a peripheral sympathetic nervous system cancer in a sample. In embodiments, the method comprises contacting the sample with the monoclonal antibody as described herein or an scFv antibody as described herein; and detecting the presence or absence of an antibody-antigen complex, thereby detecting the presence of ALK in the sample.

In embodiments, the contacting comprises immunohistochemistry. For example, immunohistochemistry comprises precipitation, immunofluorescence, western blot, or ELISA.

For example, the sample can be whole blood, a blood component, a body fluid, a biopsy, a tissue, serum or one or more cells. For example, the sample comprises a normal sample or a cancerous sample. For example, the tissue comprises brain tissue or nervous system tissue. For example, the body fluid comprises pleural fluid, peritoneal fluid, CSF, or urine.

In embodiments, the one or more cells comprise an in vitro culture.

In embodiments, the one or more cells comprise ALK-expressing cells.

In embodiments, the sample is an in vitro sample.

Embodiments can further comprise a step of obtaining a sample from a subject.

In embodiments, the sample can be a cancer sample. For example, the cancer expresses ALK, such as full-length ALK. For example, the cancer comprises neuroblastoma, glioma, rhabdomyosarcoma, non-small cell lung cancer, inflammatory myofibroblastic tumors, anaplastic large cell lymphoma, or thyroid cancer.

Aspects of the invention are also drawn towards a method of treating cancer. For example, the cancer comprises neuroblastoma, glioma, rhabdomyosarcoma, non-small cell lung cancer, inflammatory myofibroblastic tumors, anaplastic large cell lymphoma, or thyroid cancer. In embodiments, the method comprises contacting a sample with a monoclonal antibody or scFv antibody as described herein; detecting the presence or absence of an antibody-antigen complex, wherein the presence of an antibody-antigen complex indicates the presence of cancer in the subject; and administering to the subject an anticancer agent, thereby treating cancer in the subject.

In embodiments, the contacting comprises immunohistochemistry, such as immunoprecipitation, immunofluorescence, western blot, ELISA.

In embodiments, the sample is whole blood, a blood component, a body fluid, a biopsy, a tissue, serum or one or more cells. For example, the one or more cells comprise ALK-expressing cells.

In embodiments, the sample comprises a normal sample or a cancerous sample.

In embodiments, the tissue comprises brain tissue or nervous system tissue.

In embodiments, the body fluid comprises pleural fluid, peritoneal fluid, CSF, or urine.

In embodiments, the one or more cells comprise an in vitro culture.

In embodiments, the sample is an in vitro sample.

Embodiments further comprise the step of obtaining a sample from a subject, such as a cancer sample. In embodiments, the cancer expresses ALK, such as full-length ALK.

Embodiments can also comprise administering to a subject in need thereof a therapeutically effective amount of a composition comprising an antibody or scFv as described herein or the engineered cell comprising a CAR as described herein.

Embodiments can further comprise administering to the subject an anti-cancer agent.

Still further, aspects of the invention are drawn to a method of decreasing metastasis in a subject. For example, the method comprises administering to a subject in need thereof a therapeutically effective amount of a composition comprising an antibody or scFv as described herein, or the engineered cell comprising a CAR.

In embodiments, the subject is afflicted with a cancer, such as a cancer that expresses ALK, for example full-length ALK.

Other objects and advantages of this invention will become readily apparent from the ensuing description.

### BRIEF DESCRIPTION OF THE FIGURES

FIG. 1A shows ALK undergoes extracellular cleavage in NB cells at Asn654-Leu655. (A) Schematic illustration of domain structure of ALK. TM, transmembrane; SP, signal peptide; LDL, low-density lipoprotein receptor domain; MAM, meprin, A-5 protein, and receptor protein-tyrosine phosphatase mu domain; G-rich, glycine-rich; and PTK, protein tyrosine kinase domains. Numbers indicate amino acid positions. Dashed gray line indicates putative ligand binding region. (B) Western blot (WB) analysis of ALK expression in the indicated NB cell lysates with a C-terminal anti-ALK antibody (C26G7). WT, wild-type; M, ALK-mutated; FL, full-length ALK; CTF, membrane-bound C-terminal ALK fragment here and throughout the figure. The upper band is a non-specific band as determined by a glycosylation assay (see also, FIG. 11, panel A). (C) WB analysis of ALK expression in PDX NB models using C-terminal anti-ALK antibodies. Lanes T1 and T2 (tumor sample SJNBL046148_X1 in duplicate); lane T3 (SJNBL013761_1). (D) WB analysis of ALK expression in brain tissue from P0 (S1) and P7 (S2) mice using an N-terminal anti-ALK antibody. (E) WB analysis of ALK expression in conditioned media (CM) or cell lysates of NB cells with an N-terminal anti-ALK antibody (8G7). SF, secreted extracellular fragment. (F) Schematic illustration of the putative extracellular cleavage site of ALK inferred from LC-MS/MS analysis. The red arrow indicates the cleavage position between amino acids 654 and 655. FIG. 1F discloses SEQ ID NOS 53, 54, and 84, respectively, in order of appearance. (G) WB analysis of ALK in 293T cells engineered to express the cleaved fragment (ALK1-654) and conditioned media from Kelly NB cells (NB ALK) that endogenously express full-length, cleaved ALK) using the ALK N-terminal 8G7 antibody. Empty vector-expressing (V) and untransfected (U) 293T cells are negative controls. (H) WB analysis of ALK expression in 293T cell lysates expressing wild-type ALK (ALK WT) or engineered variants. Cells expressing the MSCV vector and ALK A638-682 were used as negative controls. (I) Cell surface protein analysis of ALK in 293T cells engineered to express the indicated WT or mutant constructs. Cell surface proteins were labeled with sulfo-NHS-SS-biotin and isolated with streptavidin beads. See also FIG. 8, FIG. 9, and FIG. 10.

FIG. 2 shows endogenous inhibition of ALK ECD cleavage leads to altered NB cell morphology. (A) Electropherogram of the region in which the ALK LF655del mutation was introduced through biallelic CRISPR-cas9 editing. FIG. 2A discloses SEQ ID NOS 85-87, respectively, in order of appearance. (B) Representative phase contrast microscopy images of NGP NB cells in which the ECD cleavage site was genomically altered using CRISPR-cas9 editing (NGP^{ALK(LF655del)}). Unedited NGP cells (NGP) and vector control (NGP^{CRISPR ctrl}) cells were used as controls. Scale bar, 25 μm. (C) WB analysis of ALK expression in NGP^{ALK(LF655del)} or NGP^{CRISPR ctrl} control cells in which ALK WT (ALK) or empty vector (MSCV) was expressed. GAPDH was used as the loading control throughout. (D) WB analysis of ALK expression in conditioned media (CM) or lysates of the indicated NGP cells. Downstream signaling in these cells are also shown. The same cell lysates were used to perform the WB analysis shown in FIG. 5F (in the same experiment); hence the ALK and GAPDH are the same for both blots. (E) Representative immunofluorescence (IF) images of ALK expression in the same cells as in (B). Nuclei were stained with DAPI. Scale bar, 10 μm. See also FIG. 11.

FIG. 3 shows inhibition of ALK ECD cleavage in NB cells suppresses migration and invasion. (A) Representative crystal violet-stained images of transwell migration assays of the indicated cells (left). Scale bar, 100 μm. Quantification of cell migration determined by counting the number of migratory cells per field and calculating the relative numbers of modified to control cells (right). Values are reported as means±SD; *P<0.05, **P<0.01, n=3. (B) Quantification of cell invasion in NGP cells in which cleavage was inhibited through genome editing (LF655del) vs. control cells (NGP ctrl) and in cells in which ALK WT (LF655del+MSCV-ALK) or vector control (LF655del+MSCV) were expressed. Invasive capacity was quantified as means±SD, n=3; *P<0.05, **P<0.01. (C) Representative phase-contrast microscopy images of wound healing in the indicated NGP cells, at 0 and 48 hr. (left). Scale bar, 500 μm. Percentage of wound closure depicted as means±SD, n=12; **P<0.01 (right). (D) Crystal violet-stained images of transwell migration of SK-N-AS NB cells expressing the indicated ALK proteins (left). Empty vector, MSCV was used as a negative control here and in (E). Scale bar, 100 μm. Quantification of cell migration (right). Values are means±SD; **P<0.01, n=3. (E) Crystal violet-stained images of transwell migration assays of CHP-212 NB cells expressing the indicated ALK proteins (left). Scale bar, 100 μm. Quantification of cell migration (right). Values are means±SD; **P<0.01, *P<0.05, n=3. See also FIG. 12 and FIG. 13

FIG. 4 shows inhibition of ALK ECD cleavage reduces NB cell migration in vivo. (A) Representative bioluminescence images (BLIs) of NOD/SCID mice 35 days after intracardiac injection of the indicated luciferase-labeled NGP NB cells (1×10⁵ cells per animal). (B) Comparison of bioluminescence in the animals described in (A) at day 20 and day 35 post-injection. Bioluminescence was quantified as photon flux per second over time. The data are presented as mean±SEM, n=7 mice per group; *P<0.05, ***P<0.001. (C) Kaplan-Meier analysis of overall survival of mice in (A). Significance was calculated by the log-rank test (P=0.006; n=7 per group). (D) Representative BLIs of NOD/SCID mice 42 days after tail-vein injection of the indicated luciferase-labeled NGP NB cells (5×10⁵ per animal) (left). Comparison of bioluminescence quantified as in (B) at day 42 post-injection (right). Data are presented as mean±SEM, *P<0.05; n=11 (NGP^{CRISPR ctrl}), n=9 (NGP^{ALK(LF655del)}). (E) Representative BLIs of NOD/SCID mice 46 days after intracardiac injection of 1×10⁵ luciferase-labeled SK-N-AS NB cells expressing ALK WT (n=5) and the ALK LF655del CSM (n=4) (left). Comparison of bioluminescence at day 46 (right). Data are presented as mean±SEM, **P<0.01.

FIG. 5 shows inhibition of ALK ECD cleavage leads to a downregulated EMT signature. (A) Volcano plot showing gene expression changes between NGP^{CRISPR ctrl} and NGP^{ALK(LF655del)} cells. Downregulated transcripts, 0.26%; upregulated transcripts, 0.21% [false discovery rate (FDR)<0.05; fold change >2). The x-axis represents the fold change in gene expression (log 2 ratio of NGP^{ALK(LF655del)} vs. NGP^{CRISPR ctrl}) and the y-axis, the −log₁₀ (adjusted P-value). Dashed red lines denote the selected 2-fold change cutoff, while the green line denotes the selected P-value cutoff. (B) GSEA plot of an EMT signature in NGP^{ALK(LF655del)} vs NGP^{CRISPR ctrl} cells. NES, normalized enrichment scores. (C) Heat map of the top 100 genes differentially expressed between NGP^{ALK(LF655del)} cells and NGP^{CRISPR ctrl} cells. The highest ranked upregulated (yellow) and downregulated (blue) genes are listed. (D) Quantitative real-time PCR (qRT-PCR) analysis of selected differentially expressed genes in NGP^{ALK(LF655del)} vs. NGP^{CRISPR ctrl} cells. Data were normalized to GAPDH. Quantification is shown as log 2 normalized fold-change. Error bars represent means±SD (n=3 replicates). (E) GO analysis of significantly up- or downregulated genes in NGP^{ALK(LF655del)} vs. NGP^{CRISPR ctrl} cells. “Negative regulation of neuron migration” comprised the most enriched category in NGP^{ALK(LF655del)} cells (>40-fold; P=3.93e-6). (F) WB analysis of ALK and the indicated EMT markers in NGP^{ALK(LF655del)} vs. NGP^{CRISPR ctrl} cells. Unedited cells (NGP) were used as a reference. GAPDH was used as a loading control.

FIG. 6 shows ALK ECD cleavage affects EMT gene expression through changes in nuclear β-catenin localization. (A) WB analysis of co-immunoprecipitated (co-IP) ALK and β-catenin in 293T cells engineered to express WT or the indicated mutant ALK constructs and immunoprecipitated with an anti-ALK C-terminal antibody. (B) WB analysis of β-catenin expression in subcellular fractions and total cell lysates of the indicated NB cells. Lamin B1 expression was used as a control for the nuclear fraction. (C) IF analysis of β-catenin expression in the indicated cells (left). Nuclei are stained with DAPI (blue). Red, β-catenin. Scale bar, 10 μm. Quantification of β-catenin signal in the nucleus (right); ****P<0.0001, Student t-test. (D) WB analysis of total and phospho-β-catenin in total cell lysates of the indicated cells. GAPDH used as a loading control. (E) ChIP-qPCR analysis of β-catenin and TCF4 occupancy at putative TCF4/LEF/TCF-4E binding sites of the indicated gene promoters in NGP CRISPR ctrl and NGP^{ALK(LF655del)} cells. Numbers on the x-axis indicate the genomic locations of the putative TCF4 sites with reference to the transcription start sites. Data represent means±SD, n=3 biological replicates (blue dots). *P<0.05; one-tailed Welch's t-test.

FIG. 7 shows ECD cleavage of ALK is mediated by MMP-9 whose inhibition suppresses cell migration. (A) WB analysis of ALK expression in lysates and CM of NGP cells treated with the broad-spectrum protease inhibitor GM6001 and the MMP-9/13 inhibitor CAS 204140-01-2, at the indicated doses, for 12 hr. Untreated (U) or DMSO-treated cells were used as controls. The 8G7 N-terminal anti-ALK antibody was used to detect the shed fragment in CM and a C-terminal anti-ALK antibody used for the cell lysates in this and subsequent panels. β-actin is used as a loading control throughout this figure. (B) WB analysis of ALK expression in NGP cells treated with the MMP-9 (CTK8G1150) and MMP-3/12/13 (MMP408) inhibitors (2 μM×12 hr.). (C) WB analysis of ALK expression in the indicated NB cell lines treated with or without CTK8G1150 (2 μM for 12 hr.). (D) WB analysis of MMP-9 and ALK expression in NGP and BE (2)-C NB cells expressing either a control shRNA (shGFP) or two different shRNAs against MMP-9. (E) Crystal violet-stained images of NGP and IMR-5 NB cells treated with CTK8G1150 (2 M) or DMSO for 16 hr. and then subjected to transwell migration assays for 48 hr. (left). Scale bar, 100 μm. Quantification of migration (right) reported as means±SD; ** p<0.01, n=4. See also, FIG. 14.

FIG. 8 shows mass spectrometry analysis of the shed ALK ectodomain fragment (see also, FIG. 1). (A) Coomassie blue-stained SDS-PAGE gel of ALK immunoprecipitated with the N-terminal 8G7 antibody from conditioned media of NGP NB cells that endogenously express wild-type ALK (left). Lane M: protein molecular weight marker; lane S: loaded sample. Arrow indicates the band cut out and subjected to mass spectrometry. WB analysis of ALK in CM from NGP cells (right). SF, secreted fragment. (B) Representative ALK peptides identified by tandem MS of chymotrypsin-digested ALK protein and subsequent database search. Chymotrypsin was used in the enzymatic digestion of the sample. The amino acid sequence of full-length ALK is shown in the single-letter code. The peptide regions identified from mass spectrometry are shown in gray. The peptide (⁶³⁶LTISGEDKILQNTAPKSRN⁶⁵⁴ (SEQ ID NO: 53)) near the cleavage site is in red, the transmembrane domain is underlined. Note that only peptides from the extracellular domain were detected. (C) Representative tandem MS spectra of the peptide identified in (B) using Scaffold software (http://www.proteomesoftware.com/products/scaffold/). (D) Representative ALK peptides identified by tandem MS of trypsin digested and database searching of the trypsin-digested fragment from conditioned media of ALK-expressing cells. The peptide (⁶⁴⁴ILQNTAPK⁶⁵¹ (SEQ ID NO: 54)) near the cleavage site is highlighted in red, and the remainder of the peptide regions are as in (B). Again, only peptides from the extracellular domain were detected. (E) Representative tandem MS spectra of the peptide identified in (D). (F) ALK protein sequence alignment of the ALK ECD using Clustal Omega (https://www.ebi.ac.uk/Tools/msa/clustalo/). Arrow points to the ECD cleavage site. FIG. 8 discloses SEQ ID NOS 56, 53, 56, 54, and 88-101, respectively, in order of appearance.

FIG. 9 shows ALK CSMs are displayed on the cell surface and inhibit ECD shedding (see also, FIG. 1). (A) Schema of mutant ALK constructs generated in this study. Arrows indicate the cleavage site. FIG. 9A discloses SEQ ID NOS 102-107, respectively, in order of appearance. (B) Immunoblot analysis of ALK cleavage in the indicated ALK variants. pcDNA3.1, vector control. (C) WB analysis of ALK in NIH3T3 cells expressing ALK WT or ALK variants. (D) Cell surface biotinylation assay of ALK WT and mutated ALK proteins expressed in 293T cells. ALK ECD del, an ALK mutant construct lacking the entire extracellular domain (without the signal peptide) acts as a negative control.

FIG. 10 shows effects of ALK cleavage inhibition on the ALK receptor (See also, FIG. 1). (A) Co-immunoprecipitation (Co-IP) analysis of ALK dimerization. WB analysis of HA-tag expression in HEK293 cells expressing WT or LF655del ALK (HA- or FLAG-tagged) singly or in combination and immunoprecipitated with anti-ALK antibodies. (B) Relative kinase activity measured by in-vitro kinase assay, of the indicated WT and mutant ALK proteins when overexpressed in NIH3T3 cells and immunoprecipitated with anti-ALK antibodies. Result are reported as means±SD (n=3 replicates). (C) WB analysis of phosphorylated and total ALK, and downstream signaling molecules in NIH3T3 cells engineered to express the indicated WT and CSM ALK constructs. (D) WB analysis of ALK and pALK in NIH3T3 cells stably expressing the indicated WT and variant ALK proteins in the presence or absence of media from cells expressing the ALKAL1/2 proteins. © Scanning electron microscope images of the Drosophila eye ectopically expressing human (ALK-WT) and CSM ALK variants (ALK LF655del) through the eye-specific GMR-GAL4 driver, in the presence or absence of the ALK ligand Alkal2. The ALK-F1174L mutation was used as the positive control.

FIG. 11 shows inhibition of ECD cleavage does not affect downstream signaling or cell proliferation (See also, FIG. 2). (A) WB analysis of ALK expression in NGP (ALK WT) and NGP^{ALK(LF655del)} (ALK LF655del) cells treated with or without PNGase F in the deglycosylation assay as described in Methods. Of the two bands at the 140 kDa marker, the lower band shows a decrease in molecular weight upon deglycosylation, hence this is the truncated ALK fragment. The upper fragment is a non-specific band. (B) ALK expression in NB cell lines. mRNA expression shown as transcripts per million (TPM). (Cancer Cell Line Encyclopedia (CCLE) https://portals.broadinstitute.org/ccle). (C) Cell surface protein analysis of ALK in SK-N-AS cells engineered to express the indicated WT and mutant constructs. Cell surface proteins were labeled with sulfo-NHS-SS-biotin and isolated with streptavidin beads. (D) WB analysis of phosphorylated and total ALK, and downstream signaling molecules in SK-N-AS (left) and CHP-212 (right) cells engineered to express the indicated WT and mutant ALK construct© (E) Growth curve of NGP, NGP^{CRISPR ctrl}, and NGP^{ALK(LF655del)} cells analyzed using the Cell Titer-Glo assay. Values are means±SD (n=3), RLU, relative light units. (F) Phase-contrast micrographs of soft agar colonies of the cells described in (C). Cells were grown for 14 days. Scale bar, 100 μm. (G) Flow cytometry analyses of propidium iodide staining in NGP^{CRISPR ctrl} and NGP^{ALK(LF655del)} cells. Percentage of cells in G1, S, and G2/M are shown.

FIG. 12 shows ALK ECD cleavage inhibition decreases cell migration and invasion of NIH3T3 but not ALK(F1174L)-mutated NB cells (See also, FIG. 3). (A) Crystal violet-stained transwell migration images of NIH3T3 cells expressing ALK WT or ALK variants (left). Cells were incubated for 16 hr. to facilitate analysis of migration. Representative fields of migratory cells on the membrane and quantification of migratory cells per field are shown. Scale bar, 150 μm. Quantification of cell migration determined by counting the number of migratory cells per field and calculating the relative numbers of modified to control cells (right). Values are means±SD, n=3; ** p<0.01. (B) Cell invasion of NIH3T3 cells expressing WT ALK or variants. Values are means±SD; n=3, ** p<0.01. (C) Electropherogram of the region in Kelly cells NB cells in which the ALK CSM was introduced through biallelic CRISPR-cas9 editing (Kelly C-35) FIG. 12C discloses SEQ ID NOS 108-112, respectively, in order of appearance. (D) WB analysis of ALK, pALK and downstream signaling in the cells in (A) with Kelly and Kelly CRISPR cells used as controls. (E) Representative crystal violet-stained images of transwell migration assays of the indicated cells (left). Scale bar, 100 μm. Quantification is shown on the right. Values are means±SD; n=3. (F) Analysis of cell invasion in the cells in (B). Invasive capacity was quantified as means±SD, n=3. (G) Representative phase-contrast microscopy images of wound healing in the indicated Kelly cells, at 0 and 48 hr. Scale bar, 500 μm. Percentage of wound closure depicted as means±SD, n=6 is shown on the right.

FIG. 13 shows individual expression of either the shed or truncated ALK fragments have no effect on cell migration (See also, FIG. 3). (A) WB analysis of the ALK ECD in the conditioned medium (left). Crystal violet-stained transwell migration images of the indicated NGP cells incubated for 24 hr. with conditioned medium containing the ALK ECD fragment (middle). Quantification of cell migration (right). Values are means±SD; n=3. Scale bar, 150 μm. (B) Crystal violet-stained transwell migration images of CHP-212 cells incubated with the indicated CM for 24 hr. (left). Quantification of cell migration (right). Values are means±SD; n=3. Scale bar, 150 μm.

FIG. 14 shows MMP-9 mediates ALK ECD cleavage (See also, FIG. 7). (A) Immunoblot analyses of ALK expression in NGP cells after shRNA depletion of ADAM17 or ADAM10. Five separate shRNAs for ADAM17 and two shRNAs for ADAM10 were used. GADPH or β-actin were included as internal loading controls in this and subsequent panels. (B) Schematic illustration of the protease predicted to cleave ALK at the identified ECD cleavage site (Asn654-Leu655) generated using PROSPER and CleavPredict software. FIG. 14B discloses SEQ ID NO: 73. (C) WB analysis of ALK expression in the indicated lysates or conditioned media from cells engineered to express WT ALK and treated with or without the MMP-9 inhibitor, CTK8G1150 (2 μM for 12 hr.). Untreated cells or cells treated with DMSO were used as controls. The 8G7 N-terminal anti-ALK antibody was used to detect the shed fragment in conditioned medium (CM) and a C-terminal anti-ALK antibody was used for the cell lysates in this and subsequent panels. (D) WB analysis of ALK expression in NGP cells treated with the indicated concentrations of CTK8G1150 for 12 hr. (E) WB analysis of ALK expression in NGP cells treated with CTK8G1150 (2 μM) for the indicated time periods. (F) WB analysis of ALK following incubation of recombinant MMP-9 to ALK protein purified from 293T cells engineered to express WT or ALK LF655del.

FIG. 15 shows the 8G7 anti-ALK antibody binds specifically to the N-terminal region of the ALK protein. (A) Western blot analysis of ALK in Kelly and SHEP neuroblastoma cells overexpressing GFP and mutated ALK using the mAb-7 and 8G7 anti-ALK antibodies. The mAb-7 antibody was developed at the Vigny laboratory (INSERM, Unité 706/Université Pierre et Marie Curie, Paris F-75005, France) (Moog-Lutz et al, 2005; PMID: 15886198) and obtained through a Materials Transfer Agreement. (B) Western blot analysis of ALK expression in various non-neuroblastoma cell lines: H1373 (human colon carcinoma), HeLa (cervical cancer), NIH-3T3 (transformed mouse fibroblast cells) using 8G7 and C26G7 (rabbit monoclonal C-terminal anti-ALK antibody; Cell Signaling Technology; Cat #3333; RRID:AB_836862) anti-ALK antibodies.

FIG. 16 shows the 8G7 anti-ALK antibody can immunoprecipitate the full-length ALK receptor. Cell lysates from the indicated neuroblastoma cell lines were subjected to immunoprecipitation (IP) using 8G7 and subsequently to immunoblotting analysis using the commercial anti-ALK C-terminal antibody. 8G7 immunoprecipitated only the full-length 220 kDa form of ALK, indicating specificity towards the N-terminal region of ALK.

FIG. 17 shows the 8G7 antibody can be used for analysis of ALK expression by immunofluorescence (IF) assays. (A) IF images (40×) of NGP neuroblastoma cells expressing wild type ALK stained with the 8G7 antibody. A 1:100 antibody dilution was used. (B) IF analysis of ALK expression using the 8G7 antibody in NIH3T3 cells engineered to express the following wild-type (NIH3T3-ALK WT) or mutant ALK proteins: NIH3T3-ALKLF655del, NIH3T3 ALK LF655FY, (cleavage-inhibited ALK) and NIH3T3-ALK-del MAM2 to 654 aa (deletion of the majority of the ECD, and hence not recognized by 8G7). The last plasmid serves as a negative control similar to NIH3T3-MSCV cells in which the vector only is expressed.

FIG. 18 shows the 8G7 antibody can be used for ELISA analysis of ALK expression in patient sera. Detection of the purified ALK-ECD protein using 8G7 (detection) and 7F7 (capture) anti-ALK antibodies by sandwich ELISA. (A) Standard curve showing the gradual increase in signal with increasing concentrations of the ALK-ECD protein in the media of NGP cells that express cleaved ALK. Quantification is shown below. (B) ELISA of the shed ALK ECD protein in sera from patients with neuroblastoma. Ample diluent buffer was used to dilute the serum samples, Media represents Dulbecco's Modified Eagle media in which human embryonic kidney 293T cells were grown, 293T media, conditioned media from 293T cells; ALK ECD 293T media, media collected from 293T cells in which the ALK-ECD-mFc protein was overexpressed.

FIG. 19 shows generation of ALK-ECD-fusion constructs. The indicated vectors were used to clone the human ALK-ECD domain that was selected as an immunogen. (A) pFUse-mIgG2A-FC2-human ALK_ECD. (B) pFUse-hIgG2-FC2-human ALK_ECD. Constructs were transfected into 293T cells to express the 150 kDa recombinant proteins (ALK-ECD-mFc and ALK-ECD-hFc) and expression was confirmed in the cell supernatants (supes) by western blotting. (C) Western blot analysis of conditioned media (CM) from 293T cells engineered to express the mIgG2A-FC2-human ALK_ECD and (D) hIgG2-FC2-human ALK_ECD plasmids. V, vector control. Sufficient protein was produced by large scale transfections of 293T cells and purified using affinity chromatography. (F) The human ALK extracellular domain (19-682 aa) fused to the mouse Fc gene was used as the immunogen to raise antibodies.

FIG. 20 shows immunization and testing of mouse sera. The purified ALK-ECD-mFc fusion protein was injected subcutaneously into three transgenic ALK knockout (homozygous null) immunocompetent mice twice a week, for a total of 8 injections followed by an intraperitoneal boost. Sera were collected at the end of the 3rd and 5th weeks and tested for their ability to kill Baf3-ALK F1174L (mouse hematopoetic cells engineered to express human ALKF1174L) cells. Viability of Baf3-ALK F1174L cells exposed to sera from mice before and after immunization with the ALK protein is shown. 1×10⁶ cells were seeded into 96-well plates and treated 24 hours later with the indicated dilutions of sera from immunized and non-immunized mice. Data were analyzed after 24 hrs using the MTT assay.

FIG. 21 shows immunization and testing of mouse sera. The purified ALK-ECD-mFc fusion protein was injected subcutaneously into three transgenic ALK knockout (homozygous null) immunocompetent mice twice a week, for a total of 8 injections followed by an intraperitoneal boost. Sera were collected at the end of the 3rd and 5th weeks and tested for their ability to recognize the ALK antigen by ELISA, WB and FACS analyses. Shown here is the FACS analysis of the binding ability of sera from mice immunized with the ALK ECD-mFc fusion protein in four different cell lines, with [SHSY5Y, NGP (human neuroblastoma) and Ba/F3ALKF1174L (mouse hematopoetic cells engineered to express human ALKF1174L)] and without (Ba/F3 MSCVVuCO) ALK expression. Based on these assays, we prioritized one mouse for hybridoma fusion with two others as back-up. Hybridomas were generated by fusing spleen cells from mouse #3 with the murine myeloma cell line Sp/20.

FIG. 22 shows generation and screening of hybridomas. HAT-selected fused cells were distributed in 96 well plates and the hybridoma supernatants were subjected to several rounds of screening. Initial screening was performed using ELISA against human Fc to test antigen binding with the ALK ECD-human Fc fusion protein. Of 800+ supernatants screened, 82 were found to be positive. These were rescreened by FACS against Ba/F3 and neuroblastoma (NB) cells expressing ALK. From this screen, 22 positive hybridoma supernatants were tested in an antigen-binding capture ELISA assay and 11 were subsequently selected for further analysis. (A) Binding of hybridoma supernatants using an antigen-binding capture assay. The plate was coated with goat-anti-mouse IgG Fc, incubated with hybridoma supernatants, followed by ALK-HRP and visualized using an HRP substrate. (B) Binding of selected hybridoma supernatants to a human Fc antibody using ELISA screening. Plates were coated with the ALK ECD-human Fc fusion protein (ALK-hFc) together with an irrelevant hFc protein and visualized using an anti-mouse kappa light chain antibody. Only the supernatants with the highest binding are shown.

FIG. 23 shows ALK-binding and -inhibitory capacity of the hybridoma supernatants. (A). Western blot analysis of the indicated hybridoma clone supernatants in NIH3T3 mouse fibroblast and NGP, SHSY5Y and Kelly neuroblastoma cells (from left to right in each of the blots). Actin as a loading control is shown on the right. The hybridoma supernatants were used as the primary antibody. (B) The effect of the supernatants on cell viability when exposed to neuroblastoma and non-neuroblastoma cells that express ALK was also tested, but they were not found to have significant direct cell killing ability. Cells were seeded at 5000 cells/well in a 96-well plate, and 24 h later treated with 100 μl of antibody supernatant. Cell viability was analyzed after 72 h using the Cell titer Glo assay (Promega). NIH3T3 cells that do not express ALK were used as a control. Percent viability is shown.

FIG. 24 shows the hybridomas were subcloned into 96 well plates by limiting dilution to ensure clonality and stability. Supernatants from individual clones were then screened by ELISA and FACS analysis for ALK binding. Shown here is the viability of NGP neuroblastoma cells (expressing ALK) in the presence of anti-mouse IgG antibody in hybridoma clone supernatants. Taken together, following 3 rounds of subcloning, one individual clone each was isolated from the 7F7, 8G7 and 5H3 supernatants for HPLC purification.

FIG. 25 shows ALK mAbs bind to ALK. The three HPLC-purified monoclonal antibodies (5H3, 7F7 and 8G7) all of the IgG1 subtype, were tested for their ability to bind to ALK in neuroblastoma cells expressing ALK. Both cell lysates and supernatants from cells grown with and without serum (to increase the expression of ALK) were tested by western blotting. All three monoclonal antibodies recognized a 220 kDa protein in the cell lysates of neuroblastoma cells and an approximately 100 kDa protein in the media which correspond to the full length ALK protein and the shed ECD domain respectively. (A) Western blot analysis of the indicated hybridoma clones in neuroblastoma cell lines expressing WT (NGP) and mutated (SHSY5Y, Kelly) ALK. NIH3T3 cells were used as controls. (B) Western blot analysis of conditioned media from NGP neuroblastoma and 293T cells engineered to express the WT ALK receptor, with the three ALK mAbs showing binding with the shed ALK extracellular domain.

FIG. 26 shows anti-ALK antibodies have minimal direct effects on cell viability. SHSY5Y neuroblastoma cells expressing ALK were treated with the monoclonal antibodies and cell growth inhibition measured. The maximum cell growth inhibition (approximately 58%) was observed only at high antibody concentrations. Moreover, there was no clear difference between ALK expressing and non-overexpressing cells. To test whether blocking mouse Fc would increase growth inhibition, cells were treated with mouse IgG Fc (30 to 60 μg/mL) prior to treatment with the ALK antibodies. This did result in some increase in cell death however this was not significant in comparison with non-neuroblastoma cell lines (A) Viability of SHSY5Y neuroblastoma cells that express ALK after 24 hr exposure to 30, 60 or 120 μg of the 8G7, 5H3 and 7F7 purified ALK mAbs. Viability measured using the CellTiter Glo assay (Promega). (B) Viability of the same cells as in (A) after 24 hr exposure to the mAbs (30 μg) with varying concentrations of the mouse IgG Fc mAb. 293T cells without ALK expression were used as controls.

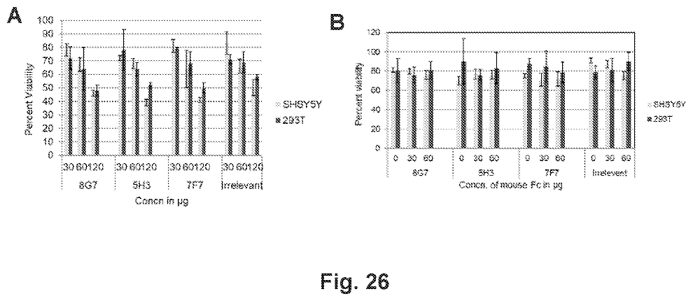

FIG. 27 shows ALK mAbs bind to the ALK receptor in immunofluorescence assays. Immunofluorescence detection of ALK with 8G7 and 7F7 antibodies in SHEP NB cells engineered to express the ALKF1174L mutation. Staining with the M13 antibody obtained from the Vigny laboratory under an MTA is shown for comparison (Moog-Lutz et al, 2005). Cells expressing the vector only (SHEP) were used as controls. Cells were stained with a 1:100 dilution of antibody. Images were captured at 40× with a Zeiss AXIO Imager Z1 fluorescence microscope.

FIG. 28 shows ALK mAbs bind to the ALK receptor in immunocyto- and immunohistochemistry assays. (A). Immunocytochemistry staining with the 8G7 antibody of 293T and NB9464 (mouse neuroblastoma cells derived from Th-MycN transgenic tumor model; Weiss et al, 1997) cells expressing a control vector or WT ALK. 5×10\hat{ }6 cells were fixed in 10% formalin and images captured using a Zeiss AXIO Imager Z1 fluorescence microscope following staining. (B) Immunohistochemistry of tumor tissue from the ALK F1174L/MYCN transgenic mouse model using the 8G7 antibody. 8G7 showed binding in engineered cells and transgenic mice tumors.

FIG. 29 shows inhibition of ALK cleavage sensitizes NB cells to retinoic acid-induced differentiation. (A) Phase contrast images of NGP, NGP^{CRISPR ctrl}, and NGP^{ALK(LF655del)} cells treated with retinoic acid (10 nM for 48 hr. followed by 48 hr. of washout) or DMSO control. Scale bar, 50 μm. (B) Quantification of neurite outgrowth (left panel) and percentage of cells with neurites (right panel) in the cells used in (A). Results are presented as means±SD (n=3). RA, retinoic acid (C) WB analysis of ALK and PRAME expression in the indicated cells. NGPALK-KO, cells in which WT ALK was depleted using CRISPR-Cas9 editing. (D) Immunofluorescence images of PRAME expression in the indicated cells. Nuclei are stained with DAPI. Scale bar, 10 μm. (E) Phase contrast microscopy images (left panel) and quantification of neurite outgrowth (right panel) of the indicated cells treated with or without RA as in (A), and engineered to express PRAME or a control vector. Quantification is presented as means±SD (n=3). (F) Top panel, WB analysis of PRAME expression in NGP cells expressing shRNAs against PRAME or GFP. Two separate shRNAs were used. Bottom panel, Quantification of neurite outgrowth of the same cells following treatment with RA (as in (A). The data are reported as means±SD (n=4).

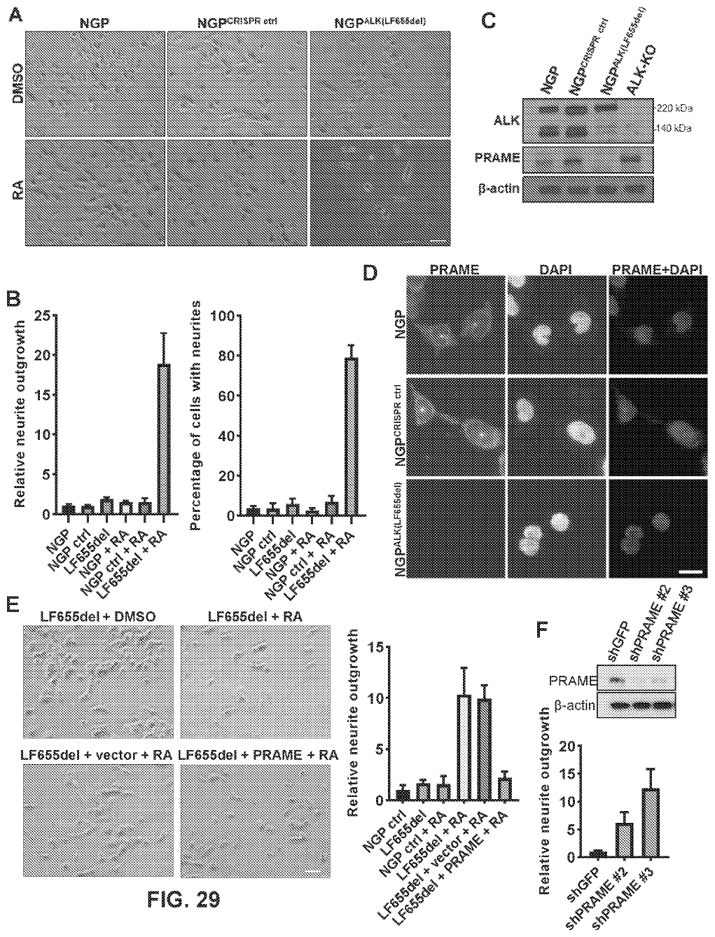

FIG. 30 shows endogenous inhibition of ECD cleavage does not affect cell proliferation. (A) Electropherogram of the region in which the ALK LF655del mutation was introduced through biallelic CRISPR-cas9 editing. FIG. 30A discloses SEQ ID NOS 85-87, respectively, in order of appearance. (B) WB analysis of phosphorylated and total downstream signaling molecules in NGP, NGP^{CRISPR ctrl}, and NGP^{ALK(LF655del)} cells. (C) Growth curve of NGP, NGP^{CRISPR ctrl}, and NGP^{ALK(LF655del)} cells as analyzed using the Cell Titer-Glo assay. Values are means±SD (n=3), RLU, relative light units. (D) Representative images of crystal violet-stained NGP^{CRISPR ctrl} and NGP^{ALK(LF655del)} cells analyzed for colony formation assays for 14 days. (E) Phase-contrast micrographs of soft agar colonies of the cells described in (D). Cells were allowed to grow for 14 days. Scale bar, 100 μm. (F) Heat map of the top 100 genes differentially expressed between NGP^{ALK(LF655del)} cells and NGP^{CRISPR ctrl} cells. The highest ranked upregulated (yellow) and downregulated (blue) genes are listed. (G) Quantitative real-time PCR (qRT-PCR) analysis of the indicated genes in NGP^{ALK(LF655del)} vs. NGP^{CRISPR ctrl} cells. Data were normalized to GAPDH. Relative quantities were showed as log 2 normalized fold change. Error bars represent means±SD (n=3 replicates). (H) GSEA plot an epithelial-to-mesenchymal transition signature NGP^{ALK(LF655del)} cells vs. NGP^{CRISPR ctrl} cells.

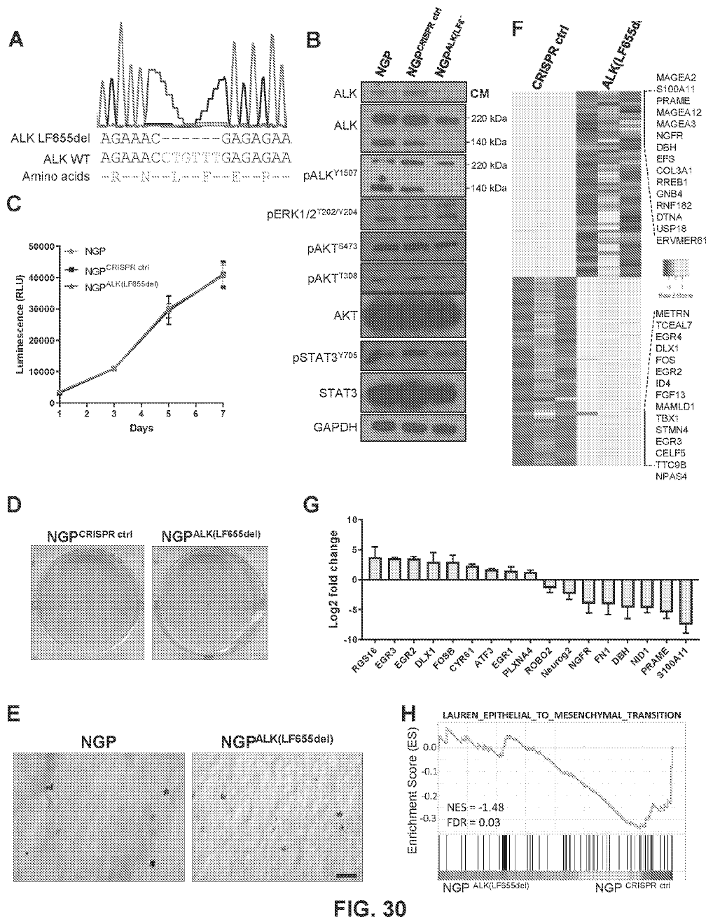

FIG. 31 shows ALK cleavage affects NIH3T3 cell migration and invasion. (A) Left panel, Transwell migration assay of NIH3T3 cells expressing ALK or ALK variants. Cells were incubated for 16 hr. to facilitate analysis of migration. Representative fields of migratory cells on the membrane and quantification of migratory cells per field are shown. Scale bar, 100 μm. Right panel, Quantification of cell migration determined by counting the number of migratory cells per field, and calculating the relative numbers of modified to control cells. Values are means±SD, n=3; ** p<0.01. (B) Cell invasion of NIH3T3 cells expressing WT ALK or variants. Values are means±SD; n=3, ** p<0.01. (C) Wound healing of NIH3T3 cells expressing wild-type or mutant ALK constructs. Representative images show the scratch (wound) at 0 and 24 hours. Scale bar, 500 μm.

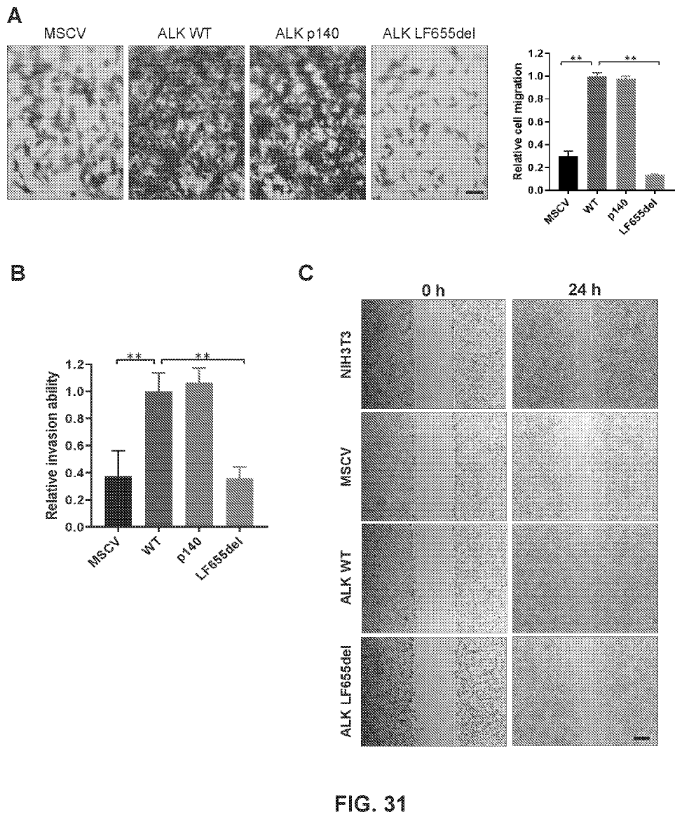

FIG. 32 shows PRAME expression correlated with a poor outcome in NB patients. (A) Kaplan-Meier analysis of overall survival probability in NB patients based on PRAME expression levels. Results generated using the R2 microarray analysis and visualization platform (r2.amc.nl). Results from four different datasets are shown. (B) Volcano plot of gene expression changes in NGP^{ALK(LF655del)} vs. NGP^{CRISPR ctrl} cells. The top three significantly downregulated genes are depicted in red. Dashed red lines denote the selected 2-fold change cutoff, while the green line denotes the selected P-value cutoff. (C) WB analysis of the indicated proteins in cell lysates from NGP^{ALK(LF655del)} cells in which HA-tagged PRAME was re-expressed.

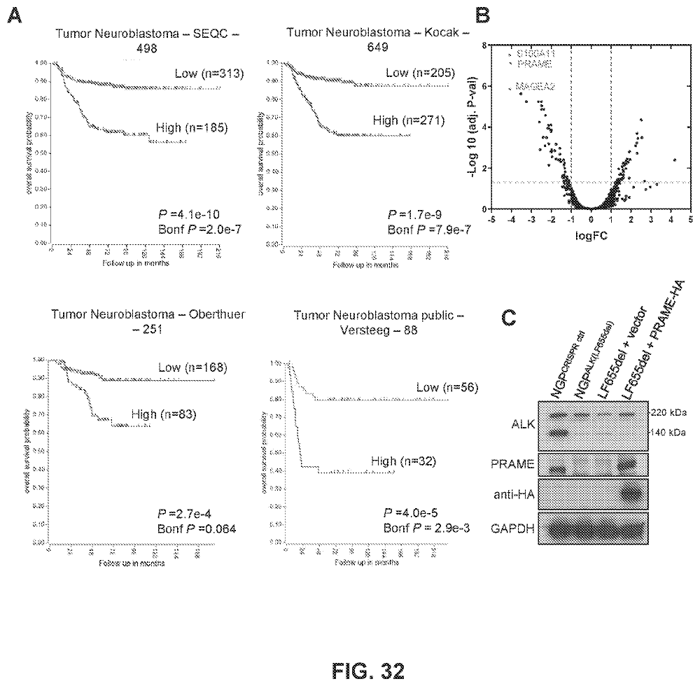

### DETAILED DESCRIPTION

Anti-ALK antibodies are provided. The antibodies can detect the ALK protein in neuroblastoma and other cancer cell lines and tumor samples in which the full-length receptor is expressed using a variety of techniques, including, immunoblotting, immunofluorescence and immunoprecipitation. The antibodies can detect the cleaved/shed ALK extracellular domain in serum samples of patients with full-length ALK-expressing tumors. Thus, the clinical utility of these antibodies includes, but is not limited to, the diagnosis of full-length ALK expressing tumors, for monitoring response to treatment and early identification of tumor progression and/or relapse. These antibodies also can be utilized in various forms of immunotherapy in combination with other targeted agents.

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### ALK

Anaplastic lymphoma kinase (ALK) is a receptor tyrosine kinase that was first identified in a chromosomal translocation associated with anaplastic large cell lymphoma (ALCL), a subtype of T-cell non-Hodgkin's lymphoma. Chromosomal translocations involving the kinase domain of ALK are seen in many cancers. In addition to ALCL, ALK fusion proteins are seen in diffuse large B-cell lymphoma (DLBCL), inflammatory myofibroblastic tumor (IMT), breast cancer, colorectal cancer, esophageal squamous cell cancer (ESCC), renal cell cancer (RCC), and non-small-cell lung cancer (NSCLC). ALK fusion partners drive dimerization of the ALK kinase domain, leading to autophosphorylation, which in turn causes the kinase to become constitutively active.

Oncogenic ALK can also be expressed due to point mutations in the kinase domain as is seen in neuroblastoma (NB), where germline mutations in ALK have been documented to drive the majority of hereditary neuroblastoma cases and for worsened prognosis when present somatically in primary tumors (Chen, et al. (2008). Oncogenic mutations of ALK kinase in neuroblastoma. Nature 455, 971-974; George, et al. (2008). Activating mutations in ALK provide a therapeutic target in neuroblastoma. Nature 455, 975-978; Janoueix-Lerosey, I et al. (2008). Somatic and germline activating mutations of the ALK kinase receptor in neuroblastoma. Nature 455, 967-970; Mosse, et al. (2008). Identification of ALK as a major familial neuroblastoma predisposition gene. Nature 455, 930-935). Constitutively active oncogenic ALK signals through multiple pathways, including PI3K/AKT, RAS/ERK, and JAK/STAT3; this signaling leads to enhanced cell proliferation and survival.

ALK inhibitors have been developed as anti-cancer agents, including the FDA approved ALK inhibitors crizotinib and ceritinib. Although the development of these inhibitors for mutationally-activated ALK has been an important step in the treatment of neuroblastomas with ALK mutations, these ALK mutations are notably found to be present in only 10% of neuroblastomas. By contrast, wild-type ALK is expressed in more than 90% of neuroblastomas, and is phosphorylated and thus activated in the majority. In addition, in advanced or metastatic neuroblastoma, high levels of ALK expression, rather than ALK mutation, is most strongly associated with poor outcome. Indeed, inhibition of wild-type ALK in some of these neuroblastoma cell lines arrests growth and leads to cell death (Mosse, et al. (2008) Identification of ALK as a major familial neuroblastoma predisposition gene. Nature 455, 930-935; Passoni, L., et al. (2009). Mutation-independent anaplastic lymphoma kinase overexpression in poor prognosis neuroblastoma patients. Cancer research 69, 7338-7346). Together with the observation that neuroblastoma possesses a surprisingly low number of genetic mutations, an alternative, DNA mutation-independent mechanism drives tumorigenesis in these neuroblastomas.

Analysis of tumor samples and neuroblastoma cell lines revealed that ALK is expressed as a 220 kDa full-length receptor and a shorter 140 kDa isoform. This finding indicated that the extracellular portion of ALK can be cleaved, leading to the shedding of the ALK ectodomain. As described herein, a monoclonal antibody specific for the shed ALK ectodomain is generated and characterized. The antibodies described herein can be used as a laboratory tool for multiple applications, such as but not limited 1 to immunofluorescence, western blotting, immunoprecipitation, and ELISA. The antibodies also have clinical utility in detecting levels of cleaved ALK as a biomarker in the serum of neuroblastoma patients, which could be directly leveraged as a diagnostic tool to detect tumor presence, tumor relapse or response to chemotherapy. In addition, without wishing to be bound by theory, the antibodies described herein can have therapeutic utility, for example by mediating complement activation against human neuroblastoma cells in vitro, as well as an ability to reduce tumor growth in a murine model of neuroblastoma when administered in vivo. In embodiments, these antibodies can be utilized as part of immunotherapy applications in combination with other targeted agents, such as MMP inhibitors to inhibit cleavage and thus to increase the level of the full-length receptor on the cell surface, thereby enhancing the role of ALK as a tumor-associated antigen.

Aspects of the invention provide isolated monoclonal antibodies specific to ALK. The term “isolated” as used herein with respect to cells, nucleic acids, such as DNA or RNA, can refer to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides.

Unique recombinant monoclonal ALK antibodies are described herein. These include 8G7, 5H3, and 7F7. “Recombinant” as it pertains to polypeptides (such as antibodies) or polynucleotides can refer to a form of the polypeptide or polynucleotide that does not exist naturally, a non-limiting example of which can be created by combining polynucleotides or polypeptides that would not normally occur together.

The nucleic acid and amino acid sequence of the monoclonal ALK antibodies are provided below; the amino acid sequences of the heavy and light chain complementary determining regions (CDRs) of the ALK antibodies are underlined (CDR1), underlined and bolded (CDR2), or underlined, italicized, and bolded (CDR3) below:

TABLE 1A
Ab 8G7 Variable Region nucleic acid sequences

V_{H} chain of Ab 8G7 (SEQ ID NO: 1)
caggttcaactgcagcagtctggggctgagctggtgaggc
ctggggcttcagtgacgctgtcctgcaaggcttcgggcta
cacatttactgactatgaaatgcactgggtgaagcagaca
cctgtgcatggcctggaatggattggagctattgatcctg
aaactggtggttctgcctacaatcagaagttcaaggccaa
ggccatactgactgcagacagatcctccagcacagcctac
atggagctccgcagcctgacatctgaggactctgccgtct
attactgtacaagaactcgttactacggaagtagtcctgc
tatggactactggggtcaaggaacctcagtcaccgtctcc
tca
V_{L} chain of Ab 8G7 (SEQ ID NO: 2)
gatatccagatgacacagactacatcctccctgtctgcct
ctctgggagacagagtcaccatcagttgcagtgcaagtca
gggcattggcaattatttaaactggtatcagcagaaacca
gatggaactgttaaactcctgatctattacacatcaagtt
tacactcaggagtcccatcaaggttcagtggcagtgggtc
tgggacagattattctctcaccatcagcaacctggaacct
gaagatattgccacttactattgtcagcagtatagtaagc
ttccgtacacgttcggaggggggaccaagctggaaataaa
a

TABLE 1B
Ab 8G7 Variable Region amino acid sequences

V_{H} chain of Ab 8G7 (SEQ ID NO: 3)
QVQLQQSGAELVRPGASVTLSCKASGYTFTDYEMHWVKQTPVHGLEWIGAIDPETGGSAYNQKF
KAKAILTADRSSSTAYMELRSLTSEDSAVYYCTRTRYYGSSPAMDYWGQGTSVTVSS
V_{L} chain of Ab 8G7 (SEQ ID NO: 4)
DIQMTQTTSSLSASLGDRVTISCSASQGIGNYLNWYQQKPDGTVKLLIYYTSSLHSGVPSRFSG
SGSGTDYSLTISNLEPEDIATYYCQQYSKLPYTFGGGTKLEIK

TABLE 2A
Ab 5H3 Variable Region nucleic acid sequences

V_{H} chain of Ab 5H3 (SEQ ID NO: 11)
atggaatggagctgggtctttctcttcctcctgtcagtaattgcaggtgtccaatcccaggttc
aactgcagcagtctggggctgagctggtgaggcctggggcttcagtgacgctgtcctgcaaggc
ttcgggctacacatttactgactatgaaatgcactgggtgaagcagacacctgtgcatggcctg
gaatggattggagctattgatcctgaaactggtggttctgcctacaatcagaagttcaaggcca
aggccatactgactgcagacagatcctccagcacagcctacatggagctccgcagcctgacatc
tgaggactctgccgtctattactgtacaagaactcgttactacggaagtagtcctgctatggac
tactggggtcaaggaacctcagtcaccgtctcctcagccaaaacgacacccccatctgtctatc
cactggcccctggatctgctgcccaaactaactccatggtgaccctgggatgcctggtcaaggg
ctatttccctgagccagtgacagtgacctggaactctggatccctgtccagcggtgtgcacacc
ttcccagctgtcctgcagtctgacctctacactctgagcagctcagtgactgtcccctccagc
V_{L} chain of Ab 5H3(SEQ ID NO: 12)
atgtcctctgctcagttccttggtctcctgttgctctgttttcaaggtaccagatgtgatatcc
agatgacacagactacatcctccctgtctgcctctctgggagacagagtcaccatcagttgcag
tgcaagtcagggcattggcaattatttaaactggtatcagcagaaaccagatggaactgttaaa
ctcctgatctattacacatcaagtttacactcaggagtcccatcaaggttcagtggcagtgggt
ctgggacagattattctctcaccatcagcaacctggaacctgaagatattgccacttactattg
tcagcagtatagtaagcttccgtacacgttcggaggggggaccaagctggaaataaaacgggct
gatgctgcaccaactgtatccatcttcccaccatccagtgagcagttaacatctggaggtgcct
cagtcgtgtgcttcttgaacaacttctaccccaaagacatcaatgtcaagtggaagattgatgg
cagtgaacgacaaaatggcgtcctgaacagttggactgatcaggacagcaaagacagcacctac
agcatgagcagcaccctcacgttgaccaag

TABLE 2B
Ab 5H3 Variable Region amino acid sequences

V_{H} chain of Ab 5H3(SEQ ID NO: 13)
MEWSWVFLFLLSVIAGVQSQVQLQQSGAELVRPGASVTLSCKASGYTFTDYEMHWVKQTPVHGL
EWIGAIDPETGGSAYNQKFKAKAILTADRSSSTAYMELRSLTSEDSAVYYCTRTRYYGSSPAMD
YWGQGTSVTVSSAKTTPPSVYPLAPGSAAQTNSMVTLGCLVKGYFPEPVTVTWNSGSLSSGVHT
FPAVLQSDLYTLSSSVTVPSS
V_{L} chain of Ab 5H3(SEQ ID NO: 14)
MSSAQFLGLLLLCFQGTRCDIQMTQTTSSLSASLGDRVTISCSASQGIGNYLNWYQQKPDGTVK
LLIYYTSSLHSGVPSRFSGSGSGTDYSLTISNLEPEDIATYYCQQYSKLPYTFGGGTKLEIKRA
DAAPTVSIFPPSSEQLTSGGASVVCFLNNFYPKDINVKWKIDGSERQNGVLNSWTDQDSKDSTY
SMSSTLTLTK

TABLE 3A
Ab 7F7 Variable Region nucleic acid sequences

V_{H} chain of Ab 7F7 (SEQ ID NO: 21)
atggaatggagctgggtctttctcttcctcctgtcagtaattgcaggtgtccaatcccaggtt
caactgcagcagtctggggctgagctggtgaggcctggggcttcagtgacgctgtcctgcaag
gcttcgggctacacatttactgactatgaaattcactggatgaagcagacacctgtgcatggc
ctggaatggattggaactattgatcctgaaactggtggtactgcctacaatcagaagttcaag
ggcaaggccatactgactgcagacaaatcctccagcacagcctacatggagctccgcagcctg
acatctgaggactctgccgtctattactgtacaagatgttactacggtagtagttggtacttc
gatgtctggggcacagggaccacggtcaccgtctcctcagccaaaacgacacccccatctgtc
tatccactggcccctggatctgctgcccaaactaactccatggtgaccctgggatgcctggtc
aagggctatttccctgagccagtgacagtgacctggaactctggatccctgtccagcggtgtg
cacaccttcccagctgtcctgcagtctgacctctacactctgagcagctcagtgactgtcccc
tccagc
V_{L} chain of Ab 7F7(SEQ ID NO: 22)
atgtcctctgctcagttccttggtctcctgttgctctgttttcaaggtaccagatgtgatatcc
agatgacacagactacatcctccctgtctgcctctctgggagacagagtcaccatcagttgcag
tgcaagtcagggcattagcaattatttaaactggtatcagcagaaaccagatggaactgttaaa
ctcctgatctattacacatcaagtttacactcaggagtcccatcaaggttcagtggcagtgggt
ctgggacagattattctctcaccatcagcaacctggaacttgaagatattgccacttactattg
tcagcagtatagtaaggttcctcggacgttcggtggaggcaccaagctggaaatcaaacgggct
gatgctgcaccaactgtatccatcttcccaccatccagtgagcagttaacatctggaggtgcct
cagtcgtgtgcttcttgaacaacttctaccccaaagacatcaatgtcaagtggaagattgatgg
cagtgaacgacaaaatggcgtcctgaacagttggactgatcaggacagcaaagacagcacctac
agcatgagcagcaccctcacgttgaccaag

TABLE 3B
Ab 7F7 Variable Region amino acid sequences

V_{H} chain of Ab 7F7 (SEQ ID NO: 23)
MEWSWVFLFLLSVIAGVQSQVQLQQSGAELVRPGASVTLSCKASGYTFTDYEIHWMKQTPVHGL
EWIGTIDPETGGTAYNQKFKGKAILTADKSSSTAYMELRSLTSEDSAVYYCTRCYYGSSWYFDV
WGTGTTVTVSSAKTTPPSVYPLAPGSAAQTNSMVTLGCLVKGYFPEPVTVTWNSGSLSSGVHTF
PAVLQSDLYTLSSSVTVPSS
V_{L} chain of Ab 7F7(SEQ ID NO: 24)
MSSAQFLGLLLLCFQGTRCDIQMTQTTSSLSASLGDRVTISCSASQGISNYLNWYQQKPDGTVK
LLIYYTSSLHSGVPSRFSGSGSGTDYSLTISNLELEDIATYYCQQYSKVPRTFGGGTKLEIKRA
DAAPTVSIFPPSSEQLTSGGASVVCFLNNFYPKDINVKWKIDGSERQNGVLNSWTDQDSKDSTY
SMSSTLTLTK

The amino acid sequences of the heavy and light chain complementary determining regions of the ALK antibodies are shown below:

TABLE 4A

| 8G7 CDR- | | | # of AA |
|---|---|---|---|
| VH | Nucleotide sequence | Protein Sequence | residues |
| VH CDR-1 | ggctacacatttactgactatgaaat | GYTFTDYEMH (SEQ ID | 10 |
| | gcac (SEQ ID NO: 31) | NO: 05) | |
| VH CDR-2 | gctattgatcctgaaactggtggttc | AIDPETGGSAYNQKFKA | 17 |
| | tgcctacaatcagaagttcaaggcc | (SEQ ID NO: 06) | |
| | (SEQ ID NO: 32) | | |
| VH CDR-3 | actcgttactacggaagtagtcctgc | TRYYGSSPAMDY (SEQ | 12 |
| | tatggactac (SEQ ID NO: 33) | ID NO: 07) | |

TABLE 4B

| 8G7 CDR | | | # of AA |
|---|---|---|---|
| VL | Nucleotide sequence | Protein Sequence | residues |
| VL CDR-1 | agtgcaagtcagggcattggcaatta | SASQGIGNYLN (SEQ | 11 |
| | tttaaac (SEQ ID NO: 34) | ID NO: 08) | |
| VL CDR-2 | tacacatcaagtttacactca (SEQ | YTSSLHS (SEQ ID NO: | 7 |
| | ID NO: 35) | 09) | |
| VL CDR-3 | cagcagtatagtaagcttccgtacac | QQYSKLPYT (SEQ ID | 9 |
| | g (SEQ ID NO: 36) | NO: 10) | |

TABLE 5A

| 5H3 CDR | | | # of AA |
|---|---|---|---|
| VH | Nucleotide Sequence | Protein Sequence | residues |
| VH CDR-1 | ggctacacatttactgactatgaaatg | GYTFTDYE (SEQ | 8 |
| | (SEQ ID NO: 37) | ID NO: 15) | |
| VH CDR-2 | attgatcctgaaactggtggttct | IDPETGGS (SEQ | 8 |
| | (SEQ ID NO: 38) | ID NO: 16) | |
| VH CDR-3 | acaagaactcgttactacggaagtag | IDPETGGS (SEQ | 14 |
| | tcctgctatggactac (SEQ ID | ID NO: 16) | |
| | NO: 39) | | |

TABLE 5B

| 5H3 CDR | | | # of AA |
|---|---|---|---|
| VL | Nucleotide sequence | Protein Sequence | residues |
| VL CDR-1 | cagggcattggcaattat (SEQ ID | QGIGNY (SEQ ID NO: | 6 |
| | NO: 40) | 18) | |
| VL CDR-2 | tacacatca (SEQ ID NO: 41) | YTS (SEQ ID NO: 19) | 3 |
| VL CDR-3 | cagcagtatagtaagcttccgtacac | QQYSKLPYT (SEQ ID | 9 |
| | g (SEQ ID NO: 42) | NO: 20) | |

TABLE 6A

| 7F7 CDR- | | | # of AA |
|---|---|---|---|
| VH | Nucleotide sequence | Protein Sequence | residues |
| VH CDR-1 | ggctacacatttactgactatgaa | GYTFTDYE (SEQ ID | 8 |
| | (SEQ ID NO: 43) | NO: 25) | |
| VH CDR-2 | attgatcctgaaactggtggtact | IDPETGGT (SEQ ID | 8 |
| | (SEQ ID NO: 44) | NO: 26) | |
| VH CDR-3 | acaagatgttactacggtagtagttg | TRCYYGSSWYFDV | 13 |
| | gtacttcgatgtc (SEQ ID NO: | (SEQ ID NO: 27) | |
| | 45) | | |

TABLE 6B

| 7F7 CDR | | | # of AA |
|---|---|---|---|
| VL | Nucleotide sequence | Protein Sequence | residues |
| VL CDR-1 | cagggcattagcaattat (SEQ ID | QGITNY (SEQ ID NO: | 6 |
| | NO: 46) | 28) | |
| VL CDR-2 | tacacatca (SEQ ID NO: 47) | YTS (SEQ ID NO: 29) | 3 |
| VL CDR-3 | cagcagtatagtaaggttcctcggac | QQYSKVPRT (SEQ ID | 9 |
| | g (SEQ ID NO: 48 | NO: 30) | |

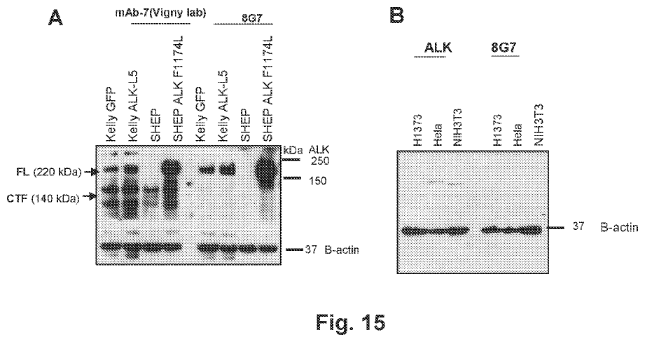

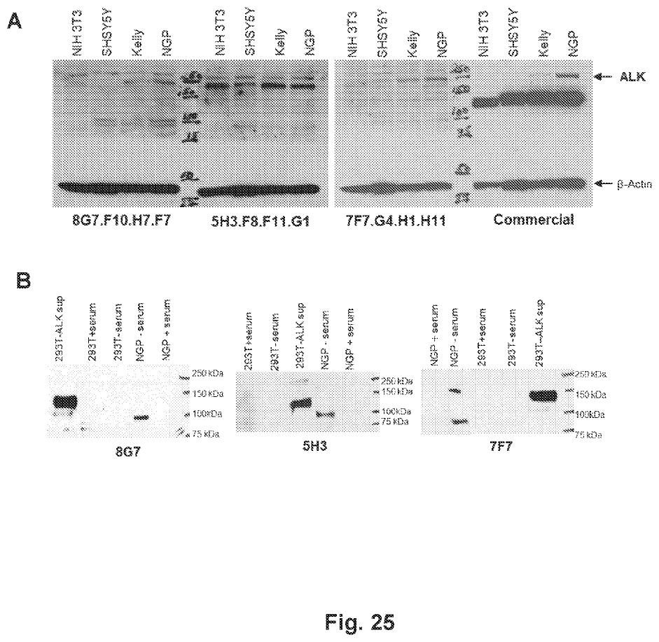

The ALK antibodies described herein bind to ALK. In one embodiment, the ALK antibodies have high affinity and high specificity for ALK. For example, referring to FIG. 15 and FIG. 25, for example, 8G7 has better specificity compared to commercial Abs, as the antibody was raised in ALK-null mice (genetically engineered mice, i.e. mice in which the ALK gene was deleted). Without wishing to be bound by theory, the improved specificity will be shared by all clones.

Embodiments also feature antibodies that have a specified percentage identity or similarity to the amino acid or nucleotide sequences of the anti-ALK antibodies described herein. For example, “homology” or “identity” or “similarity” can refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the anti-ALK antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the anti-ALK antibodies described herein. Sequence identity or similarity to the nucleic acids and proteins herein can be determined by sequence comparison and/or alignment by methods known in the art, for example, using software programs known in the art, such as those described in Ausubel et al. eds. (2007) Current Protocols in Molecular Biology. For example, sequence comparison algorithms (i.e. BLAST or BLAST 2.0), manual alignment or visual inspection can be utilized to determine percent sequence identity or similarity for the nucleic acids and proteins of the present invention.

“Polypeptide” as used herein can encompass a singular “polypeptide” as well as plural “polypeptides,” and can refer to a molecule composed of monomers (amino acids) linearly linked by amide bonds (also known as peptide bonds). The term “polypeptide” can refer to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In embodiments the alteration results in the substitution of an amino acid with a chemically similar amino acid. Conservative substitution tables providing functionally similar amino acids are well known in the art.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

### Antibodies

An “antibody” or “antigen-binding polypeptide” can refer to a polypeptide or a polypeptide complex that specifically recognizes and binds to an antigen, such as ALK. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment” can refer to a portion of an antibody such as F_{(ab′)2}, F_{(ab)2}, F_{ab}′, F_{ab}, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” can refer to a fusion protein of the variable regions of the heavy (VH) and light chains (VL) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. (See Huston et al. (1988) Proc Nat Acad Sci USA 85(16):5879-5883). In embodiments the regions are connected with a short linker peptide, such as a short linker peptide of about ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Very large naive human scFv libraries have been and can be created to offer a large source of rearranged antibody genes against a plethora of target molecules. Smaller libraries can be constructed from individuals with infectious diseases in order to isolate disease-specific antibodies. (See Barbas et al., Proc. Natl. Acad. Sci. USA 89:9339-43 (1992); Zebedee et al, Proc. Natl. Acad. Sci. USA 89:3 175-79 (1992)).

Antibody molecules obtained from humans fall into five classes of immunoglubulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” VH and VL regions, which contain the CDRs, as well as frameworks (FRs) of the ALK antibodies are shown in Table 1-Table 3.

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions can adopt a β-sheet conformation and the CDRs form loops which connect, and in some cases form part of, the β-sheet structure. The framework regions act to form a scaffold that provides for positioning the CDRs in correct orientation by interchain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” can refer to the numbering system set forth by Kabat et al., U.S. Dept. of Health and Human Services, “Sequence of Proteins of Immunological Interest” (1983).

As used herein, the term “epitope” can include any protein determinant capable of specific binding to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants usually consist of chemically active surface groupings of molecules such as amino acids or sugar side chains and usually have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3). In one embodiment, the antibodies can be directed to ALK (having Genbank accession no. [NM 004304]; 1620 amino acid residues in length), comprising the amino acid sequence of SEQ ID NO: NP_004295.

Human ALK gene seq (SEQ ID NO: 55):
ATGGGAGCCATCGGGCTCCTGTGGCTCCTGCCGCTGCTGCTTTCCACGGCAGCTGTGGGCTCCGGGATGG
GGACCGGCCAGCGCGCGGGCTCCCCAGCTGCGGGGCCGCCGCTGCAGCCCCGGGAGCCACTCAGCTACTC
GCGCCTGCAGAGGAAGAGTCTGGCAGTTGACTTCGTGGTGCCCTCGCTCTTCCGTGTCTACGCCCGGGAC
CTACTGCTGCCACCATCCTCCTCGGAGCTGAAGGCTGGCAGGCCCGAGGCCCGCGGCTCGCTAGCTCTGG
ACTGCGCCCCGCTGCTCAGGTTGCTGGGGCCGGCGCCGGGGGTCTCCTGGACCGCCGGTTCACCAGCCCC
GGCAGAGGCCCGGACGCTGTCCAGGGTGCTGAAGGGCGGCTCCGTGCGCAAGCTCCGGCGTGCCAAGCAG
TTGGTGCTGGAGCTGGGCGAGGAGGCGATCTTGGAGGGTTGCGTCGGGCCCCCCGGGGAGGCGGCTGTGG
GGCTGCTCCAGTTCAATCTCAGCGAGCTGTTCAGTTGGTGGATTCGCCAAGGCGAAGGGCGACTGAGGAT
CCGCCTGATGCCCGAGAAGAAGGCGTCGGAAGTGGGCAGAGAGGGAAGGCTGTCCGCGGCAATTCGCGCC
TCCCAGCCCCGCCTTCTCTTCCAGATCTTCGGGACTGGTCATAGCTCCTTGGAATCACCAACAAACATGC
CTTCTCCTTCTCCTGATTATTTTACATGGAATCTCACCTGGATAATGAAAGACTCCTTCCCTTTCCTGTC
TCATCGCAGCCGATATGGTCTGGAGTGCAGCTTTGACTTCCCCTGTGAGCTGGAGTATTCCCCTCCACTG
CATGACCTCAGGAACCAGAGCTGGTCCTGGCGCCGCATCCCCTCCGAGGAGGCCTCCCAGATGGACTTGC
TGGATGGGCCTGGGGCAGAGCGTTCTAAGGAGATGCCCAGAGGCTCCTTTCTCCTTCTCAACACCTCAGC
TGACTCCAAGCACACCATCCTGAGTCCGTGGATGAGGAGCAGCAGTGAGCACTGCACACTGGCCGTCTCG
GTGCACAGGCACCTGCAGCCCTCTGGAAGGTACATTGCCCAGCTGCTGCCCCACAACGAGGCTGCAAGAG
AGATCCTCCTGATGCCCACTCCAGGGAAGCATGGTTGGACAGTGCTCCAGGGAAGAATCGGGCGTCCAGA
CAACCCATTTCGAGTGGCCCTGGAATACATCTCCAGTGGAAACCGCAGCTTGTCTGCAGTGGACTTCTTT
GCCCTGAAGAACTGCAGTGAAGGAACATCCCCAGGCTCCAAGATGGCCCTGCAGAGCTCCTTCACTTGTT
GGAATGGGACAGTCCTCCAGCTTGGGCAGGCCTGTGACTTCCACCAGGACTGTGCCCAGGGAGAAGATGA
GAGCCAGATGTGCCGGAAACTGCCTGTGGGTTTTTACTGCAACTTTGAAGATGGCTTCTGTGGCTGGACC
CAAGGCACACTGTCACCCCACACTCCTCAATGGCAGGTCAGGACCCTAAAGGATGCCCGGTTCCAGGACC
ACCAAGACCATGCTCTATTGCTCAGTACCACTGATGTCCCCGCTTCTGAAAGTGCTACAGTGACCAGTGC
TACGTTTCCTGCACCGATCAAGAGCTCTCCATGTGAGCTCCGAATGTCCTGGCTCATTCGTGGAGTCTTG
AGGGGAAACGTGTCCTTGGTGCTAGTGGAGAACAAAACCGGGAAGGAGCAAGGCAGGATGGTCTGGCATG
TCGCCGCCTATGAAGGCTTGAGCCTGTGGCAGTGGATGGTGTTGCCTCTCCTCGATGTGTCTGACAGGTT
CTGGCTGCAGATGGTCGCATGGTGGGGACAAGGATCCAGAGCCATCGTGGCTTTTGACAATATCTCCATC
AGCCTGGACTGCTACCTCACCATTAGCGGAGAGGACAAGATCCTGCAGAATACAGCACCCAAATCAAGAA
ACCTGTTTGAGAGAAACCCAAACAAGGAGCTGAAACCCGGGGAAAATTCACCAAGACAGACCCCCATCTT
TGACCCTACAGTTCATTGGCTGTTCACCACATGTGGGGCCAGCGGGCCCCATGGCCCCACCCAGGCACAG
TGCAACAACGCCTACCAGAACTCCAACCTGAGCGTGGAGGTGGGGAGCGAGGGCCCCCTGAAAGGCATCC
AGATCTGGAAGGTGCCAGCCACCGACACCTACAGCATCTCGGGCTACGGAGCTGCTGGCGGGAAAGGCGG
GAAGAACACCATGATGCGGTCCCACGGCGTGTCTGTGCTGGGCATCTTCAACCTGGAGAAGGATGACATG
CTGTACATCCTGGTTGGGCAGCAGGGAGAGGACGCCTGCCCCAGTACAAACCAGTTAATCCAGAAAGTCT
GCATTGGAGAGAACAATGTGATAGAAGAAGAAATCCGTGTGAACAGAAGCGTGCATGAGTGGGCAGGAGG
CGGAGGAGGAGGGGGTGGAGCCACCTACGTATTTAAGATGAAGGATGGAGTGCCGGTGCCCCTGATCATT
GCAGCCGGAGGTGGTGGCAGGGCCTACGGGGCCAAGACAGACACGTTCCACCCAGAGAGACTGGAGAATA
ACTCCTCGGTTCTAGGGCTAAACGGCAATTCCGGAGCCGCAGGTGGTGGAGGTGGCTGGAATGATAACAC
TTCCTTGCTCTGGGCCGGAAAATCTTTGCAGGAGGGTGCCACCGGAGGACATTCCTGCCCCCAGGCCATG
AAGAAGTGGGGGTGGGAGACAAGAGGGGGTTTCGGAGGGGGTGGAGGGGGGTGCTCCTCAGGTGGAGGAG
GCGGAGGATATATAGGCGGCAATGCAGCCTCAAACAATGACCCCGAAATGGATGGGGAAGATGGGGTTTC
CTTCATCAGTCCACTGGGCATCCTGTACACCCCAGCTTTAAAAGTGATGGAAGGCCACGGGGAAGTGAAT
ATTAAGCATTATCTAAACTGCAGTCACTGTGAGGTAGACGAATGTCACATGGACCCTGAAAGCCACAAGG
TCATCTGCTTCTGTGACCACGGGACGGTGCTGGCTGAGGATGGCGTCTCCTGCATTGTGTCACCCACCCC
GGAGCCACACCTGCCACTCTCGCTGATCCTCTCTGTGGTGACCTCTGCCCTCGTGGCCGCCCTGGTCCTG
GCTTTCTCCGGCATCATGATTGTGTACCGCCGGAAGCACCAGGAGCTGCAAGCCATGCAGATGGAGCTGC
AGAGCCCTGAGTACAAGCTGAGCAAGCTCCGCACCTCGACCATCATGACCGACTACAACCCCAACTACTG
CTTTGCTGGCAAGACCTCCTCCATCAGTGACCTGAAGGAGGTGCCGCGGAAAAACATCACCCTCATTCGG
GGTCTGGGCCATGGCGCCTTTGGGGAGGTGTATGAAGGCCAGGTGTCCGGAATGCCCAACGACCCAAGCC
CCCTGCAAGTGGCTGTGAAGACGCTGCCTGAAGTGTGCTCTGAACAGGACGAACTGGATTTCCTCATGGA
AGCCCTGATCATCAGCAAATTCAACCACCAGAACATTGTTCGCTGCATTGGGGTGAGCCTGCAATCCCTG
CCCCGGTTCATCCTGCTGGAGCTCATGGCGGGGGGAGACCTCAAGTCCTTCCTCCGAGAGACCCGCCCTC
GCCCGAGCCAGCCCTCCTCCCTGGCCATGCTGGACCTTCTGCACGTGGCTCGGGACATTGCCTGTGGCTG
TCAGTATTTGGAGGAAAACCACTTCATCCACCGAGACATTGCTGCCAGAAACTGCCTCTTGACCTGTCCA
GGCCCTGGAAGAGTGGCCAAGATTGGAGACTTCGGGATGGCCCGAGACATCTACAGGGCGAGCTACTATA
GAAAGGGAGGCTGTGCCATGCTGCCAGTTAAGTGGATGCCCCCAGAGGCCTTCATGGAAGGAATATTCAC
TTCTAAAACAGACACATGGTCCTTTGGAGTGCTGCTATGGGAAATCTTTTCTCTTGGATATATGCCATAC
CCCAGCAAAAGCAACCAGGAAGTTCTGGAGTTTGTCACCAGTGGAGGCCGGATGGACCCACCCAAGAACT
GCCCTGGGCCTGTATACCGGATAATGACTCAGTGCTGGCAACATCAGCCTGAAGACAGGCCCAACTTTGC
CATCATTTTGGAGAGGATTGAATACTGCACCCAGGACCCGGATGTAATCAACACCGCTTTGCCGATAGAA
TATGGTCCACTTGTGGAAGAGGAAGAGAAAGTGCCTGTGAGGCCCAAGGACCCTGAGGGGGTTCCTCCTC
TCCTGGTCTCTCAACAGGCAAAACGGGAGGAGGAGCGCAGCCCAGCTGCCCCACCACCTCTGCCTACCAC
CTCCTCTGGCAAGGCTGCAAAGAAACCCACAGCTGCAGAGATCTCTGTTCGAGTCCCTAGAGGGCCGGCC
GTGGAAGGGGGACACGTGAATATGGCATTCTCTCAGTCCAACCCTCCTTCGGAGTTGCACAAGGTCCACG
GATCCAGAAACAAGCCCACCAGCTTGTGGAACCCAACGTACGGCTCCTGGTTTACAGAGAAACCCACCAA
AAAGAATAATCCTATAGCAAAGAAGGAGCCACACGACAGGGGTAACCTGGGGCTGGAGGGAAGCTGTACT
GTCCCACCTAACGTTGCAACTGGGAGACTTCCGGGGGCCTCACTGCTCCTAGAGCCCTCTTCGCTGACTG
CCAATATGAAGGAGGTACCTCTGTTCAGGCTACGTCACTTCCCTTGTGGGAATGTCAATTACGGCTACCA
GCAACAGGGCTTGCCCTTAGAAGCCGCTACTGCCCCTGGAGCTGGTCATTACGAGGATACCATTCTGAAA
AGCAAGAATAGCATGAACCAGCCTGGGCCCTGA

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the equilibrium binding constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361: 186-87 (1993)). The ratio of K_{off}/Kₒₙ enables the cancellation of all parameters not related to affinity, and is equal to the equilibrium binding constant, K_{D}. (See, generally, Davies et al. (1990) Annual Rev Biochem 59:439-473). An antibody of the present invention can specifically bind to a ALK epitope when the equilibrium binding constant (K_{D}) is ≤1 μM, ≤10 μM, ≤10 nM, ≤10 pM, or ≤100 pM to about 1 pM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

For example, the ALK antibody can be monovalent or bivalent, and comprises a single or double chain. Functionally, the binding affinity of the ALK antibody is within the range of 10⁻⁵M to 10⁻¹² M. For example, the binding affinity of the ALK antibody is from 10⁻⁶M to 10⁻¹² M, from 10⁻⁷M to 10⁻¹² M, from 10⁻⁸ M to 10⁻¹² M, from 10⁻⁹M to 10⁻¹² M, from 10⁻⁵ M to 10⁻¹¹ M, from 10⁻⁶ M to 10⁻¹¹ M, from 10⁻⁷M to 10⁻¹¹ M, from 10⁻⁸ M to 10⁻¹¹ M, from 10⁻⁹M to 10⁻¹¹ M, from 10⁻¹⁰ M to 10⁻¹¹ M, from 10⁻⁵ M to 10⁻¹⁰M, from 10⁻⁶ M to 10⁻¹⁰ M, from 10⁻⁷M to 10⁻¹⁰ M, from 10⁻⁸ M to 10⁻¹⁰M, from 10⁻⁹M to 10⁻¹⁰ M, from 10⁻⁵ M to 10⁻⁹M, from 10⁻⁶M to 10° M, from 10⁻⁷M to 10⁻⁹M, from 10⁻⁸ M to 10⁻⁹M, from 10⁻⁵ M to 10⁻⁸M, from 10⁻⁶M to 10⁻⁸ M, from 10⁻⁷M to 10⁻⁸ M, from 10⁻⁵ M to 10⁻⁷M, from 10⁻⁶ M to 10⁻⁷M, or from 10⁻⁵ M to 10⁻⁶ M.

An ALK protein of the invention, or a derivative, fragment, analog, homolog or ortholog thereof, can be utilized as an immunogen in the generation of antibodies that immunospecifically bind these protein components. For example, a polypeptide according to NP_004295 or a fragment thereof.

The amino acid sequence for ALK protein comprises:

| SEQ ID NO: 56 | |
|---|---|
| 1 | mgaigllwll pllistaavg sgmgtgqrag spaagpplqp replsysrlq rkslavdfvv |
| 61 | pslfrvyard lllppsssel kagrpeargs laldcapllr llgpapgvsw tagspapaea |
| 121 | rtlsrvlkgg svrklrrakq lvlelgeeai legcvgppge aavgllqfnl selfswwirq |
| 181 | gegrlrirlm pekkasevgr egrlsaaira sqprllfqif gtghsslesp tnmpspspdy |
| 241 | ftwnltwimk dsfpflshrs ryglecsfdf pceleysppl hdlrnqswsw rripseeasq |
| 301 | mdlldgpgae rskemprgsf lllntsadsk htilspwmrs ssehctlavs vhrhlqpsgr |
| 361 | yiaqllphne aareillmpt pgkhgwtvlq grigrpdnpf rvaleyissg nrslsavdff |
| 421 | alkncsegts pgskmalqss ftcwngtvlq lgqacdfhqd caqgedesqm crklpvgfyc |
| 481 | nfedgfcgwt qgtlsphtpq wqvrtlkdar fqdhqdhall lsttdvpase satvtsatfp |
| 541 | apiksspcel rmswlirgvl rgnvslvlve nktgkeqgrm vwhvaayegl slwqwmvlpl |
| 601 | ldvsdrfwlq mvawwgqgsr aivafdnisi sldcyltisg edkilqntap ksrnlfernp |
| 661 | nkelkpgens prqtpifdpt vhwlfttcga sgphgptqaq cnnayqnsnl svevgsegpl |
| 721 | kgiqiwkvpa tdtysisgyg aaggkggknt mmrshgvsvl gifnlekddm lyilvgqqge |
| 781 | dacpstnqli qkvcigennv ieeeirvnrs vhewaggggg gggatyvfkm kdgvpvplii |
| 841 | aaggggrayg aktdtfhper lennssvlgl ngnsgaaggg ggwndntsll wagkslqega |
| 901 | tgghscpqam kkwgwetrgg fggggggcss ggggggyigg naasnndpem dgedgvsfis |
| 961 | plgilytpal kvmeghgevn ikhylncshc evdechmdpe shkvicfcdh gtvlaedgvs |
| 1021 | civsptpeph lplslilsvv tsalvaalvl afsgimivyr rkhqelqamq melqspeykl |
| 1081 | sklrtstimt dynpnycfag ktssisdlke vprknitlir glghgafgev yegqvsgmpn |
| 1141 | dpsplqvavk tlpevcseqd eldflmeali iskfnhqniv rcigvslqsl prfillelma |
| 1201 | ggdlksflre trprpsqpss lamldllhva rdiacgcqyl eenhfihrdi aarnclltcp |
| 1261 | gpgrvakigd fgmardiyra syyrkggcam lpvkwmppea fmegiftskt dtwsfgvllw |
| 1321 | eifslgympy psksnqevle fvtsggrmdp pkncpgpvyr imtqcwqhqp edrpnfaiil |
| 1381 | erieyctqdp dvintalpie ygplveeeek vpvrpkdpeg vppllvsqqa kreeerspaa |
| 1441 | ppplpttssg kaakkptaae isvrvprgpa vegghvnmaf sqsnppselh kvhgsrnkpt |
| 1501 | slwnptygsw ftekptkknn piakkephdr gnlglegsct vppnvatgrl pgasllleps |
| 1561 | sltanmkevp lfrlrhfpcg nvnygyqqqg lpleaatapg aghyedtilk sknsmnqpgp |

In embodiments, the immunogen comprises:

(SEQ ID NO: 57)
MGAIGLLWLLPLLLSTAAVGSGMGTGQRAGSPAAGPPLQPREPLSYSRL
QRKSLAVDFVVPSLFRVYARDLLLPPSSSELKAGRPEARGSLALDCAPL
LRLLGPAPGVSWTAGSPAPAEARTLSRVLKGGSVRKLRRAKQLVLELGE
EAILEGCVGPPGEAAVGLLQFNLSELFSWWIRQGEGRLRIRLMPEKKAS
EVGREGRLSAAIRASQPRLLFQIFGTGHSSLESPTNMPSPSPDYFTWNL
TWIMKDSFPFLSHRSRYGLECSFDFPCELEYSPPLHDLRNQSWSWRRIP
SEEASQMDLLDGPGAERSKEMPRGSFLLLNTSADSKHTILSPWMRSSSE
HCTLAVSVHRHLQPSGRYIAQLLPHNEAAREILLMPTPGKHGWTVLQGR
IGRPDNPFRVALEYISSGNRSLSAVDFFALKNCSEGTSPGSKMALQSSF
TCWNGTVLQLGQACDFHQDCAQGEDESQMCRKLPVGFYCNFEDGFCGWT
QGTLSPHTPQWQVRTLKDARFQDHQDHALLLSTTDVPASESATVTSATF
PAPIKSSPCELRMSWLIRGVLRGNVSLVLVENKTGKEQGRMVWHVAAYE
GLSLWQWMVLPLLDVSDRFWLQMVAWWGQGSRAIVAFDNISISLDCYLT
ISGEDKILQNTAPKSRNLFERNPNKELKPGENSPRQTPIFDPTVH

In embodiments, the immunogen comprises the extracellular domain.

An ALK protein or a derivative, fragment, analog, homolog, or ortholog thereof, coupled to a proteoliposome can be utilized as an immunogen in the generation of antibodies that immunospecifically bind these protein components.

Those skilled in the art will recognize that one can determine, without undue experimentation, if a human monoclonal antibody has the same specificity as a human monoclonal antibody of the invention by ascertaining whether the former prevents the latter from binding to ALK. For example, if the human monoclonal antibody being tested competes with the human monoclonal antibody of the invention, as shown by a decrease in binding by the human monoclonal antibody of the invention, then the two monoclonal antibodies can bind to the same, or to a closely related, epitope.

Another way to determine whether a human monoclonal antibody has the specificity of a human monoclonal antibody of the invention is to pre-incubate the human monoclonal antibody of the invention with the ALK protein, with which it is normally reactive, and then add the human monoclonal antibody being tested to determine if the human monoclonal antibody being tested is inhibited in its ability to bind ALK. If the human monoclonal antibody being tested is inhibited then, it can have the same, or functionally equivalent, epitopic specificity as the monoclonal antibody of the invention. Screening of human monoclonal antibodies of the invention can be also carried out by utilizing ALK and determining whether the test monoclonal antibody is able to neutralize ALK.

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen, which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with an epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes can be used if cells of human origin are desired, or spleen cells or lymph node cells can be used if non-human mammalian sources are desired. The lymphocytes are then fused with an immortalized cell line using a suitable fusing agent, such as polyethylene glycol, to form a hybridoma cell (See Goding, Monoclonal Antibodies: Principles and Practice, Academic Press, (1986) pp. 59-103). Immortalized cell lines can be transformed mammalian cells, particularly myeloma cells of rodent, bovine and human origin. For example, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines that are useful are those that fuse efficiently, support stable high-level expression of antibody by the selected antibody-producing cells, and are sensitive to a medium such as HAT medium. For example, immortalized cell lines can be murine myeloma lines, which can be obtained, for instance, from the Salk Institute Cell Distribution Center (San Diego, California) and the American Type Culture Collection (Manassas, Virginia). Human myeloma and mouse-human heteromyeloma cell lines also have been described for the production of human monoclonal antibodies. (See Kozbor, J. Immunol, 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567 (incorporated herein by reference in its entirety). DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (See U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies are antibody molecules in which the entire sequence of both the light chain and the heavy chain, including the CDRs, arise from human genes. Such antibodies are termed “human antibodies” or “fully human antibodies” herein. Human monoclonal antibodies can be prepared by using trioma technique; the human Bcell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 7796). Human monoclonal antibodies may be utilized and may be produced by using human hybridomas (see Cote, et al., 1983. Proc Natl Acad Sci USA 80: 20262030) or by transforming human B cells with Epstein Barr Virus in vitro (see Cole, et al., 1985 In: MONOCLONAL ANTIBODIES AND CANCER THERAPY, Alan R. Liss, Inc., pp. 7796).

“Humanized antibodies” can be antibodies from non-human species (such as a mouse) whose light chain and heavy chain protein sequences have been modified to increase their similarity to antibody variants produced in humans. Humanized antibodies are antibody molecules derived from a non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen-binding. These framework substitutions are identified by methods well known in the art, e.g., by modeling of the interactions of the CDR and framework residues to identify framework residues important for antigen-binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) For example, the non-human part of the antibody (such as the CDR(s) of a light chain and/or heavy chain) can bind to the target antigen.

Antibodies can be humanized using a variety of techniques known in the art including, for example, CDR-grafting (EP 239,400; PCT publication WO) 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Natl. Sci. USA 91:969-973 (1994)), and chain shuffling (U.S. Pat. No. 5,565,332, which is incorporated by reference in its entirety). “Humanization” (also called Reshaping or CDR-grafting) is a well-established technique understood by the skilled artisan for reducing the immunogenicity of monoclonal antibodies (mAbs) from xenogeneic sources (commonly rodent) and for improving their activation of the human immune system (See, for example, Hou S, Li B, Wang L, Qian W, Zhang D, Hong X, Wang H, Guo Y (July 2008). “Humanization of an anti-CD34 monoclonal antibody by complementarity-determining region grafting based on computer-assisted molecular modeling”. J Biochem. 144 (1): 115-20). Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an inexpensive production alternative to existing mammalian systems. For example, the transgenic plant may be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the invention further provides any cell or plant comprising a vector that encodes an antibody of the invention, or produces an antibody of the invention.

Human monoclonal antibodies, such as fully human and humanized antibodies, can be prepared by using trioma technique; the human B-cell hybridoma technique (see Kozbor, et al, 1983 Immunol Today 4: 72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al, 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using other techniques, including phage display libraries. (See Hoogenboom and Winter, J. Mol. Biol, 227:381 (1991); Marks et al., J. Mol. Biol, 222:581 (1991)). Human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio Technology 10, 779-783 (1992); Lonberg et al, Nature 368, 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies can additionally be produced using transgenic nonhuman animals which are modified so as to produce fully human antibodies rather than the animal's endogenous antibodies in response to challenge by an antigen. (See, PCT publication no. WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. A non-limiting example of such a nonhuman animal is a mouse, and is termed the Xenomouse™ as disclosed in PCT publication nos. WO96/33735 and WO96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO) 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, NY) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody described herein, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, is disclosed in PCT publication No. WO99/53049.

The antibody of interest can also be expressed by a vector containing a DNA segment encoding the single chain antibody described herein.

These vectors can include liposomes, naked DNA, adjuvant-assisted DNA, gene gun, catheters, etc. Vectors can further include chemical conjugates such as described in WO 93/64701, which has targeting moiety (e.g. a ligand to a cellular surface receptor), and a nucleic acid binding moiety (e.g. polylysine), viral vectors (e.g. a DNA or RNA viral vector), fusion proteins such as described in PCT/US 95/02140 (WO) 95/22618), which is a fusion protein containing a target moiety (e.g. an antibody specific for a target cell) and a nucleic acid binding moiety (e.g. a protamine), plasmids, phage, viral vectors, etc. The vectors can be chromosomal, non-chromosomal or synthetic. Retroviral vectors can also be used, and include moloney murine leukemia viruses.

DNA viral vectors can also be used, and include pox vectors such as orthopox or avipox vectors, herpesvirus vectors such as a herpes simplex I virus (HSV) vector (See Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87: 1149 (1990), Adenovirus Vectors (see LeGal LaSalle et al, Science, 259:988 (1993); Davidson, et al, Nat. Genet 3:219 (1993); Yang, et al, J. Virol. 69:2004 (1995) and Adeno-associated Virus Vectors (see Kaplitt, M. G., et al, Nat. Genet. 8: 148 (1994).

Pox viral vectors introduce the gene into the cell's cytoplasm. Avipox virus vectors result in only a short-term expression of the nucleic acid. Adenovirus vectors, adeno-associated virus vectors, and herpes simplex virus (HSV) vectors can be used for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al, Proc. Natl. Acad. Sci. USA 91:2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways, for example, to detect the presence of ALK in a sample. The antibody can also be used to try to bind to and disrupt an ALK activity. In an embodiment, the antibodies of the invention are full-length antibodies, containing an Fc region similar to wild-type Fc regions that bind to Fc receptors. In other embodiments, the antibodies of the invention are antibody fragments, such as scFv antibodies.

Techniques can be adapted for the production of single-chain antibodies specific to an antigenic protein of the invention (See e.g., U.S. Pat. No. 4,946,778). In addition, methods can be adapted for the construction of F_{ab} expression libraries (See e.g., Huse, et al, 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen can be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fv fragments.

Heteroconjugate antibodies are also within the scope of the present invention. Heteroconjugate antibodies are composed of two covalently joined antibodies. Such antibodies can, for example, target immune system cells to unwanted cells (see U.S. Pat. No. 4,676,980), and for treatment of infection (See PCT Publication Nos. WO91/00360; WO92/20373). The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The antibody of the invention can be modified with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176: 1 191-1 195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al, Anti-Cancer Drug Design, 3: 219-230 (1989)).

In certain embodiments, an antibody of the invention can comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter halt-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be desired include those applications in which localization to the brain, kidney, and/or liver is desired. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In embodiments, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In embodiments, mutations are introduced to the constant regions of the mAb such that the antibody dependent cell-mediated cytotoxicity (ADCC) activity of the mAb is altered. For example, the mutation is a LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In embodiments, antibodies of the invention for use in the diagnostic and treatment methods described herein have a constant region, e.g., an IgG₁ or IgG₄ heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In one embodiment, the antibody comprises an Fc variant with an amino acid substitution at amino acid position 228 or 299 (EU numbering). In more particular embodiments, the antibody comprises an IgG1 or IgG4 constant region comprising an S228P and a T299A mutation (EU numbering).

Exemplary amino acid substitutions which confer reduced or altered glycosylation are described in PCT Publication No, WO05/018572, which is incorporated by reference herein in its entirety. In some embodiments, the antibodies of the invention, or fragments thereof, are modified to eliminate glycosylation. Such antibodies, or fragments thereof, can be referred to as “agly” antibodies, or fragments thereof, (e.g. “agly” antibodies). While not wishing to be bound by theory “agly” antibodies, or fragments thereof, can have an improved safety and stability profile in vivo. Exemplary agly antibodies, or fragments thereof, comprise an aglycosylated Fc region of an IgG₄ antibody which is devoid of Fc-effector function thereby eliminating the potential for Fc mediated toxicity to the normal vital tissues and cells that express ALK. In yet other embodiments, antibodies of the invention, or fragments thereof, comprise an altered glycan. For example, the antibody can have a reduced number of fucose residues on an N-glycan at Asn297 of the Fc region, i.e., is afucosylated. In another embodiment, the antibody can have an altered number of sialic acid residues on the N-glycan at Asn297 of the Fc region.

The invention also is directed to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238: 1098 (1987). Carbon-14-labeled l-isothiocyanatobenzyl-3-methyldiethylene triaminepentaacetic acid (MX-DTPA) is an exemplary chelating agent for conjugation of radionucleotide to the antibody. (See POT Publication No. WO94/11026, and U.S. Pat. No. 5,736,137).

Those of ordinary skill in the art understand that a large variety of moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133: 1335-2549 (1984); Jansen et al., Immunological Reviews 62: 185-216 (1982); and Vitetta et al, Science 238: 1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of the invention include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al, Proc. Natl. Acad. Sci. USA, 82: 3688 (1985); Hwang et al, Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Non-limiting examples of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al, J. Biol. Chem., 257: 286-288 (1982) via a disulfide-interchange reaction.

### Multispecific Antibodies

Multispecific antibodies are antibodies that can recognize two or more different antigens. For example, a bi-specific antibody (bsAb) is an antibody comprising two variable domains or scFv units such that the resulting antibody recognizes two different antigens. For example, a trispecific antibody (tsAb) is an antibody comprising two variable domains or scFv units such that the resulting antibody recognizes three different antigens. The invention provides for multispecific antibodies, such as bi-specific antibodies that recognize ALK and a second antigen. For example, ALK is a tumor antigen. As a tumor antigen targeting molecule, an antibody or antigen-binding fragment specific to ALK can be combined with a second antigen-binding fragment specific to an immune cell to generate a bispecific antibody. In embodiments, the immune cell is selected from the group consisting of a T cell, a B cell, a monocyte, a macrophage, a neutrophil, a dendritic cell, a phagocyte, a natural killer cell, an eosinophil, a basophil, and a mast cell. Molecules on the immune cell which can be targeted include, but not limited to, for example, CD3, CD16, CD19, CD28, and CD64. Other non-limiting examples include PD-1, CTLA-4, LAG-3 (also known as CD223), CD28, CD122, 4-1BB (also known as CD137), TIM3, OX-40 or OX40L, CD40 or CD40L, LIGHT, ICOS/ICOSL, GITR/GITRL, TIGIT, CD27, VISTA, B7H3, B7H4, HEVM or BTLA (also known as CD272), killer-cell immunoglobulin-like receptors (KIRs), and CD47. Exemplary second antigens include tumor associated antigens (e.g., LINGO1, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin), cytokines (e.g., IL-2, IL-3, IL-4, IL-5, IL-6, IL-7, IL-10, IL-12, IL-13, IL-15, GM-CSF, TNF-α, CD40L, OX40L, CD27L, CD30L, 4-1BBL, LIGHT and GITRL), and cell surface receptors. Different formats of bispecific antibodies are also provided herein. In embodiments, each of the anti-ALK fragment and the second fragment is each independently selected from a Fab fragment, a single-chain variable fragment (scFv), or a single-domain antibody. In embodiments, the bispecific antibody further includes a Fc fragment. A bi-specific antibody of the present invention comprises a heavy chain and a light chain combination or scFv of the ALK antibodies disclosed herein.

Multispecific antibodies (e.g., bispecific antibodies and trispecific antibodies) of the invention can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds. In some embodiments, the amino acid linker (GGGGSGGGGS; “(G4S)2” (SEQ ID NO: 58)) that can be used with scFv fusion constructs described herein can be generated with a longer G4S (SEQ ID NO: 59) linker to improve flexibility. For example, the linker can also be “(G4S)3” (e.g., GGGGSGGGGSGGGGS) (SEQ ID NO: 60); “(G4S)₄” (e.g., GGGGSGGGGSGGGGSGGGGS) (SEQ ID NO: 61); “(G4S)5” (e.g., GGGGSGGGGSGGGGSGGGGSGGGGS) (SEQ ID NO: 62); “(G4S)6” (e.g., GGGGSGGGGGGGGSGGGGSGGGGSGGGGS) (SEQ ID NO: 63); “(G4S)7” (e.g., GGGGSGGGGSGGGGSGGGGSGGGGSGGGGSGGGGS) (SEQ ID NO: 64); and the like. For example, use of the (G4S)5 (SEQ ID NO: 62) linker can provide more flexibility and can improve expression. In some embodiments, the linker can also be (GS)ₙ (SEQ ID NO: 65), (GGS)ₙ (SEQ ID NO: 66), (GGGS)ₙ (SEQ ID NO: 67), (GGSG)ₙ (SEQ ID NO: 68), (GGSGG)ₙ (SEQ ID NO: 69), or (GGGGS)ₙ (SEQ ID NO: 70), wherein n is 1, 2, 3, 4, 5, 6, 7, 8, 9, or 10. Non-limiting examples of linkers known to those skilled in the art that can be used are described in U.S. Pat. No. 9,708,412; U.S. Patent Application Publication Nos. US 20180134789 and US 20200148771; and PCT Publication No. WO2019051122 (each of which are incorporated by reference in their entireties).

In embodiments, the multispecific antibodies (e.g., bispecific antibodies and trispecific antibodies) can be constructed using the “knob into hole” method (Ridgway et al, Protein Eng 7:617-621 (1996)). In this method, the Ig heavy chains of the two different variable domains are reduced to selectively break the heavy chain pairing while retaining the heavy-light chain pairing. The two heavy-light chain heterodimers that recognize two different antigens are mixed to promote heteroligation pairing, which is mediated through the engineered “knob into holes” of the CH3 domains.

In embodiments, multispecific antibodies (e.g., bispecific antibodies and trispecific antibodies) can be constructed through exchange of heavy-light chain dimers from two or more different antibodies to generate a hybrid antibody where the first heavy-light chain dimer recognizes ALK and the second heavy-light chain dimer recognizes a second antigen. In some embodiments, the bi-specific antibody can be constructed through exchange of heavy-light chain dimers from two or more different antibodies to generate a hybrid antibody where the first heavy-light chain dimer recognizes a second antigen and the second heavy-light chain dimer recognizes ALK. The mechanism for heavy-light chain dimer is similar to the formation of human IgG₄, which also functions as a bispecific molecule. Dimerization of IgG heavy chains is driven by intramolecular force, such as the pairing the CH3 domain of each heavy chain and disulfide bridges. Presence of a specific amino acid in the CH3 domain (R409) has been shown to promote dimer exchange and construction of the IgG₄ molecules. Heavy chain pairing is also stabilized further by interheavy chain disulfide bridges in the hinge region of the antibody. Specifically, in IgG₄, the hinge region contains the amino acid sequence Cys-Pro-Ser-Cys (SEQ ID NO: 71) (in comparison to the stable IgG1 hinge region which contains the sequence Cys-Pro-Pro-Cys (SEQ ID NO: 72)) at amino acids 226-230. This sequence difference of Serine at position 229 has been linked to the tendency of IgG₄ to form intrachain disulfides in the hinge region (Van der Neut Kolfschoten, M. et al, 2007, Science 317: 1554-1557 and Labrijn, A. F. et al, 2011, Journal of Immunol 187:3238-3246).

Therefore, bi-specific antibodies of the invention can be created through introduction of the R409 residue in the CH3 domain and the Cys-Pro-Ser-Cys (SEQ ID NO: 71) sequence in the hinge region of antibodies that recognize ALK or a second antigen, so that the heavy-light chain dimers exchange to produce an antibody molecule with one heavy-light chain dimer recognizing ALK and the second heavy-light chain dimer recognizing a second antigen, wherein the second antigen is any antigen disclosed herein. Known IgG₄ molecules can also be altered such that the heavy and light chains recognize ALK or a second antigen, as disclosed herein. Use of this method for constructing the bi-specific antibodies of the invention can be beneficial due to the intrinsic characteristic of IgG₄ molecules wherein the Fc region differs from other IgG subtypes in that it interacts poorly with effector systems of the immune response, such as complement and Fc receptors expressed by certain white blood cells. This specific property makes these IgG₄-based bi-specific antibodies attractive for therapeutic applications, in which the antibody is required to bind the target(s) and functionally alter the signaling pathways associated with the target(s), however not trigger effector activities.

In embodiments, mutations are introduced to the constant regions of the bsAb such that the antibody dependent cell-mediated cytotoxicity (ADCC) activity of the bsAb is altered. For example, the mutation is a LALA mutation in the CH2 domain. In one aspect, the bsAb contains mutations on one scFv unit of the heterodimeric bsAb, which reduces the ADCC activity. In another aspect, the bsAb contains mutations on both chains of the heterodimeric bsAb, which completely ablates the ADCC activity. For example, the mutations introduced one or both scFv units of the bsAb are LALA mutations in the CH2 domain. These bsAbs with variable ADCC activity can be optimized such that the bsAbs exhibits maximal selective killing towards cells that express one antigen that is recognized by the bsAb, however exhibits minimal killing towards the second antigen that is recognized by the bsAb.

The bi-specific antibodies disclosed herein can be useful in treatment of medical conditions, for example cancer.

### Use of Antibodies Against ALK

ALK is an attractive target for cancer therapies not only for its prominent role in a number of malignancies, but also for its scant expression in normal adult tissue, which is restricted to a small subset of neural cells, reducing off-target toxicities of ALK-selective agents. There are currently four FDA approved kinase inhibitors for the treatment of ALK-positive NSCLC: crizotinib, ceritinib (LDK378), alectinib, and brigatinib. ALK-positive tumors are highly sensitive to ALK inhibition, indicating that these tumors are addicted to ALK kinase activity. However, despite initial dramatic responses of variable median duration (10.9 months for crizotinib; 16.6 months for ceritinib; 25.7 months for alectinib), resistance to therapy typically develops. Moreover, to date, crizotinib, which has been tested in neuroblastoma patients has not shown durable responses.

Antibodies of the invention specifically binding an ALK protein, or a fragment thereof, can be administered for the treatment of an ALK associated disease or disorder. An “ALK-associated disease or disorder” includes disease states and/or symptoms associated with a disease state, where increased levels of ALK and/or activation of cellular signaling pathways involving ALK are found. Exemplary ALK-associated diseases or disorders include, but are not limited to cell-proliferative diseases, such as cancer.

Antibodies of the invention, including monoclonal, polyclonal, bi-specific, humanized and fully human antibodies, and fragments can be used as therapeutic agents. Such agents will generally be employed to treat or prevent cancer in a subject. An antibody preparation, for example, one having high specificity and high affinity for its target antigen, is administered to the subject and will generally have an effect due to its binding with the target. Administration of the antibody can abrogate or inhibit or interfere with an activity of the ALK protein.

The term “subject” or “patient” can refer to any organism to which aspects of the invention can be administered, e.g., for experimental, diagnostic, prophylactic, research and/or therapeutic purposes. For example, subjects to which compounds of the present disclosure can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

In embodiments, a subject comprises a mammal, such as a human or vertebrate animal. Examples of such include but are not limited to a dog, cat, horse, cow, pig, sheep, goat, chicken, primate, e.g., monkey, fish (aquaculture species), e.g. salmon, rat, and mouse. A human comprises a preterm neonate, an infant, a child, an adolescent, an adult, or an elderly individual.

Although aspects of the invention as described herein relate to human cell proliferative disorders, aspects of the invention are also applicable to other nonhuman vertebrates. Aspects of the invention are applicable for veterinary use, such as with domestic animals. Aspects will vary according to the type of use and mode of administration, as well as the particularized requirements of individual subjects.

Antibodies of the invention specifically binding an ALK protein or fragment thereof can be administered for the prevention or treatment of a cancer in the form of pharmaceutical compositions. Principles and considerations involved in preparing therapeutic pharmaceutical compositions comprising the antibody, as well as guidance in the choice of components are provided, for example, in: Remington: The Science And Practice Of Pharmacy 20th ed. (Alfonso R. Gennaro, et al, editors) Mack Pub. Co., Easton, Pa., 2000; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

The antibodies (also referred to herein as “agents of the invention” or “active compounds”) and derivatives, fragments, analogs and homologs thereof, can be incorporated into pharmaceutical compositions suitable for administration. Such pharmaceutical compositions can comprise the antibody or agent and a pharmaceutically acceptable carrier. As used herein, the term “pharmaceutically acceptable carrier” can include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Embodiments can include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. For example, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form as used herein can refer to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

A specific dosage and treatment regimen for any particular patient will depend upon a variety of factors, such as the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

A therapeutically effective amount of an antibody of the invention can be the amount needed to achieve a therapeutic objective. As noted herein, this can be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. The dosage administered to a subject (e.g., a patient) of the antigen-binding polypeptides described herein is typically 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90: 7889-7893 (1993)). The formulation can also contain more than one active compound as necessary for the particular indication being treated, such as those with complementary activities that do not adversely affect each other. Alternatively, or in addition, the composition can comprise an agent that enhances its function, such as, for example, a cytotoxic agent, cytokine (e.g. IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration can be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

Aspects of the invention comprise measuring or detecting biomarkers of a cell proliferative disease, such as cancer, in a biological sample, and thereby measuring response to treatment or disease progression over time. Biomarkers of the invention can be measured in different types of biological samples. Non-limiting examples of biological samples that can be used in methods of the invention, although not intended to be limiting, include stool, plasma, cord blood, neonatal blood, cerebral spinal fluid, tears, vomit, saliva, urine, feces, and meconium. If desired, a sample can be prepared to enhance detectability of the biomarkers. For example, a sample from the subject can be fractionated. Any method that enriches for a biomarker polypeptide of interest can be used. Sample preparations, such as prefractionation protocols, are optional and may or may not be necessary to enhance detectability of biomarkers depending on the methods of detection used. For example, sample preparation can be unnecessary if an antibody that specifically binds a biomarker is used to detect the presence of the biomarker in a sample. Sample preparation can involve fractionation of a sample and collection of fractions determined to contain the biomarkers. Methods of prefractionation include, for example, size exclusion chromatography, ion exchange chromatography, heparin chromatography, affinity chromatography, sequential extraction, gel electrophoresis, mass spectrometry, and liquid chromatography.

The methods described herein can involve obtaining a biological sample from the subject. As used herein, the phrase “obtaining a biological sample” can refer to any process for directly or indirectly acquiring a biological sample from a subject. For example, a biological sample can be obtained (e.g., at a point-of-care facility, such as a physician's office, a hospital, laboratory facility) by procuring a tissue or fluid sample (e.g., blood draw, marrow sample, spinal tap) from a subject. Alternatively, a biological sample can be obtained by receiving the biological sample (e.g., at a laboratory facility) from one or more persons who procured the sample directly from the subject. The biological sample can be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

An antibody according to the invention can be used as an agent for detecting the presence of ALK (or a protein fragment thereof) in a biological sample. For example, an embodiment can comprise the early detection of cancer relapse or recurrence, prior to radiographic scans. For example, the antibody can contain a detectable label. Antibodies can be polyclonal, monoclonal, or a fragment. An intact antibody, or a fragment thereof (e.g., F_{ab}, scFv, or F_{(ab)2}) can be used. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA includes Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations.

Procedures for conducting immunoassays are described, for example in “ELISA: Theory and Practice: Methods in Molecular Biology”, Vol. 42, J. R. Crowther (Ed.) Human Press, Totowa, N J, 1995; “Immunoassay”, E. Diamandis and T. Christopoulus, Academic Press, Inc., San Diego, (A, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Antibodies directed against an ALK protein (or a fragment thereof) can be used in methods known within the art relating to the localization and/or quantitation of an ALK protein (e.g., for use in measuring levels of the ALK protein within appropriate physiological samples, for use in diagnostic methods, for use in imaging the protein, and the like). In a given embodiment, antibodies specific to an ALK protein, or derivative, fragment, analog or homolog thereof, that contain the antibody derived antigen binding domain, are utilized as pharmacologically active compounds (referred to herein as “therapeutics”).

An antibody of the invention specific for an ALK protein can be used to isolate an ALK polypeptide by standard techniques, such as immunoaffinity, chromatography or immunoprecipitation. Antibodies directed against an ALK protein (or a fragment thereof) can be used diagnostically to monitor protein levels in tissue as part of a clinical testing procedure, e.g., to, for example, determine the efficacy of a given treatment regimen.

Detection can be facilitated by coupling (i.e., physically linking) the antibody to a detectable substance. Examples of detectable substances include, but are not limited to, various enzymes, prosthetic groups, fluorescent materials, luminescent materials, bioluminescent materials, and radioactive materials. Non-limiting examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³²P or ³H.

### Methods of Treatment

As used herein, the terms “treat” or “treatment” can refer to both therapeutic treatment and prophylactic or preventative measures, wherein the object is to prevent or slow down (lessen) an undesired physiological change or disorder, such as the progression of cancer. Beneficial or desired clinical results can include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented.

The invention provides for both prophylactic and therapeutic methods of treating a subject at risk of (or susceptible to) a cancer, or other cell proliferation-related diseases or disorders. Such diseases or disorders include but are not limited to, e.g., those diseases or disorders associated with aberrant expression of ALK. For example, the methods are used to treat, prevent or alleviate a symptom of cancer. In an embodiment, the methods are used to treat, prevent or alleviate a symptom of a solid tumor. Non-limiting examples of other tumors that can be treated by embodiments herein comprise lung cancer, ovarian cancer, prostate cancer, colon cancer, cervical cancer, brain cancer, thyroid cancer, skin cancer, liver cancer, pancreatic cancer or stomach cancer, neuroblastoma, rhabdomyosarcoma. Additionally, the methods of the invention can be used to treat hematologic cancers such as leukemia and lymphoma. Alternatively, the methods can be used to treat, prevent or alleviate a symptom of a cancer that has metastasized. For example, the cancer can be neuroblastoma.

In embodiments, the invention provides for methods for preventing, treating or alleviating a symptom cancer or a cell proliferative disease or disorder in a subject by administering to the subject a monoclonal antibody, scFv antibody or bi-specific antibody of the invention or a composition comprising the same. For example, an anti-ALK antibody can be administered in therapeutically effective amounts.

Subjects at risk for cancer or cell proliferation-related diseases or disorders can include patients who have a family history of cancer or a subject exposed to a known or suspected cancer-causing agent. Administration of a prophylactic agent can occur prior to the manifestation of cancer such that the disease is prevented or, alternatively, delayed in its progression.

In another aspect, tumor cell growth is inhibited by contacting a cell with an anti-ALK antibody of the invention. The cell can be any cell that expresses ALK.

### Combinatory Methods

Compositions of the invention as described herein can be administered in combination with a chemotherapeutic agent. Chemotherapeutic agents that can be administered with the compositions described herein include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide). In addition, the antibody can be combined with targeted agents such as ALK and other receptor tyrosine kinase inhibitors, MMP-9 inhibitors, epigenetic agents and immunotherapy agents such as checkpoint inhibitors.

In embodiments, the compositions of the invention as described herein can be administered in combination with cytokines. Cytokines that may be administered with the compositions include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions described herein can be administered in combination with other therapeutic or prophylactic regimens, such as, for example, radiation therapy.

In some embodiments, the compositions described herein can be administered in combination with other immunotherapeutic agents. Non-limiting examples of immunotherapeutic agents include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8.

The invention provides for methods of treating cancer in a patient by administering two antibodies that bind to the same epitope of the ALK protein or, alternatively, two different epitopes of the ALK protein. Alternatively, the cancer can be treated by administering a first antibody that binds to ALK and a second antibody that binds to a protein other than ALK. In other embodiments, the cancer can be treated by administering a bispecific antibody that binds to ALK and that binds to a protein other than ALK. For example, the other protein other than ALK can include, but is not limited to, GD2. For example, the other protein other than ALK is a tumor-associated antigen; the other protein other than ALK can also be a cytokine.

In embodiments, the invention provides for the administration of an anti-ALK antibody alone or in combination with an additional antibody that recognizes another protein other than ALK, with cells that are capable of effecting or augmenting an immune response. For example, these cells can be peripheral blood mononuclear cells (PBMC), or any cell type that is found in PBMC, e.g., cytotoxic T cells, macrophages, and natural killer (NK) cells.

The invention provides administration of an antibody that binds to the ALK protein and an anti-neoplastic agent, such a small molecule, a growth factor, a cytokine, or other therapeutics including biomolecules such as peptides, peptidomimetics, peptoids, polynucleotides, lipid-derived mediators, small biogenic amines, hormones, neuropeptides, and proteases. Small molecules include, but are not limited to, inorganic molecules and small organic molecules. Suitable growth factors or cytokines include an IL-2, GM-CSF, IL-12, and TNF-alpha. Small molecule libraries are known in the art. (See, Lam, Anticancer Drug Des., 12: 145, 1997)

### Chimeric Antigen Receptor (CAR) T-Cell Therapies

Cellular therapies, such as chimeric antigen receptor (CAR) T-cell therapies, are also provided herein. CAR T-cell therapies redirect a patient's T-cells to kill tumor cells by the exogenous expression of a CAR. A CAR can be a membrane spanning fusion protein that links the antigen recognition domain of an antibody to the intracellular signaling domains of the T-cell receptor and co-receptor. A suitable cell can be used, that is put in contact with an anti-ALK antibody of the invention (or alternatively engineered to express an anti-ALK antibody as described herein). Solid tumors offer unique challenges for CAR-T therapies. Unlike blood cancers, tumor-associated target proteins are overexpressed between the tumor and healthy tissue resulting in on-target/off-tumor T-cell killing of healthy tissues. Furthermore, immune repression in the tumor microenvironment (TME) limits the activation of CAR-T cells towards killing the tumor. Upon such contact or engineering, the cell can then be introduced to a cancer patient in need of a treatment. The cancer patient may have a cancer of any of the types as disclosed herein. The cell (e.g., a T cell) can be, for instance, a tumor-infiltrating T lymphocyte, a CD4+ T cell, a CD8+ T cell, or the combination thereof, without limitation. Exemplary CARS useful in aspects of the invention include those disclosed in, for example, PCT/US2015/067225 and PCT/US2019/022272, each of which are hereby incorporated by reference in their entireties.

In one embodiment, the ALK antibodies discussed herein can be used in the construction of multi-specific antibodies or as the payload for a CAR-T cell. For example, in one embodiment, the anti-ALK antibodies discussed herein can be used for the targeting of the CARS (i.e., as the targeting moiety). In another embodiment, the anti-ALK antibodies discussed herein can be used as the targeting moiety, and a different ALK antibody that targets a different epitope can be used as the payload. In another embodiment, the payload can be an immunomodulatory antibody payload. In embodiments, the ALK antibodies described herein can be used as targeting moieties in CARs (e.g., kill ALK⁺ tumor cells) or as a secreted checkpoint blockade antibody to reverse T cell exhaustion.

For example, embodiments of the invention comprise chimeric antigen receptor (CAR) comprising an intracellular signaling domain, a transmembrane domain and an extracellular domain. In embodiments, the extracellular domain is an isolated monoclonal antibody or antigen-binding fragment thereof that binds to human Anaplastic Lymphoma Kinase (ALK) protein. For example, the monoclonal antibody or fragment thereof comprises a heavy chain, light chain, or combination thereof, wherein the heavy chain comprises a CDR1, CDR2, and/or CDR3 according to Tables 4-6; and wherein the light chain comprises a CDR1, CDR2, and/or CDR3 according to Tables 4-6.

The CAR according to the invention can comprise at least one transmembrane polypeptide comprising at least one extracellular ligand-biding domain and; one transmembrane polypeptide comprising at least one intracellular signaling domain; such that the polypeptides assemble together to form a Chimeric Antigen Receptor.

The term “extracellular ligand-binding domain” as used herein can refer to an oligo- or polypeptide that is capable of binding a ligand. For example, the domain can interact with a cell surface molecule. For example, the extracellular ligand-binding domain can be chosen to recognize a ligand that acts as a cell surface marker on target cells associated with a particular disease state.

In an embodiment, the extracellular ligand-binding domain can comprise an antigen binding domain derived from an antibody against an antigen of the target. For example, the target can be ALK. Thus, the CAR can be specific for ALK. In an embodiment, said extracellular ligand-binding domain is a single chain antibody fragment (scFv) comprising the light (VL) and the heavy (VH) variable fragment of a target antigen specific monoclonal antibody joined by a flexible linker. For example, said scFv antibody is specific for ALK. It is understood, however, that binding domains other than scFv can also be used for predefined targeting of lymphocytes, such as camelid single-domain antibody fragments or receptor ligands, antibody binding domains, antibody hypervariable loops or CDRs as non limiting examples.

In embodiments said transmembrane domain comprises a stalk region between said extracellular ligand-binding domain and said transmembrane domain. The term “stalk region” can refer to any oligo- or polypeptide that functions to link the transmembrane domain to the extracellular ligand-binding domain. For example, stalk region(s) is/are used to provide more flexibility and accessibility for the extracellular ligand-binding domain. A stalk region can comprise up to 300 amino acids, such as 10 to 100 amino acids. In embodiments, the stalk region comprises 25 to 50 amino acids. Stalk region can be derived from all or part of naturally occurring molecules, such as from all or part of the extracellular region of CD8, CD4 or CD28, or from all or part of an antibody constant region. Alternatively, the stalk region can be a synthetic sequence that corresponds to a naturally occurring stalk sequence, or may be an entirely synthetic stalk sequence. In an embodiment said stalk region is a part of human CD8 alpha chain.

In embodiments, the transmembrane domain can comprise CD28.

The signal transducing domain or intracellular signaling domain of the CAR of the invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” can refer to the portion of a protein which transduces the effector signal function signal and directs the cell to perform a specialized function.

Signal transduction domain can comprise two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs (immunoreceptor tyrosine-based activation motifs). ITAMs are well defined signaling motifs found in the intracytoplasmic tail of a variety of receptors that serve as binding sites for syk/zap70 class tyrosine kinases. Examples of ITAM used in the invention can include as non limiting examples those derived from TCR zeta, FcR gamma, FcR beta, FcR epsilon, CD3 gamma, CD3 delta, CD3 epsilon, CD5, CD22, CD79a, CD79b and CD66d. In a preferred embodiment, the signaling transducing domain of the CAR can comprise the CD3 zeta signaling domain, or the intracytoplasmic domain of the Fc epsilon RI beta or gamma chains. In another preferred embodiment, the signaling is provided by CD3 zeta together with co-stimulation provided by CD28 and a tumor necrosis factor receptor (TNFr), such as 4-1BB or OX40), for example.

In embodiments, the intracellular signaling domain of the CAR of the invention comprises a co-stimulatory signal molecule. In embodiments the intracellular signaling domain contains 2, 3, 4 or more co-stimulatory molecules in tandem. A co-stimulatory molecule is a cell surface molecule other than an antigen receptor or their ligands that is required for an efficient immune response.

“Co-stimulatory ligand” can refer to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, MICB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83.

A “co-stimulatory molecule” can refer to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class 1 molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In embodiments, the choice of CD28 as a co-stimulatory domain for the CARs can be based in the fact that CD28 CARs direct an active proliferative response and enhance effector functions, whereas 4-1BB-based CARs induce a more progressive T cell accumulation that may counterweigh for less immediate effectiveness. In one embodiment, the CD28 is replaced by 41BB in the CAR constructs.

In an embodiment, said signal transducing domain is a TNFR-associated Factor 2 (TRAF2) binding motifs, intracytoplasmic tail of costimulatory TNFR member family. Cytoplasmic tail of costimulatory TNFR family member contains TRAF2 binding motifs consisting of the major conserved motif (P/S/A)X(Q/E)E) or the minor motif (PXQXXD), wherein X is any amino acid. TRAF proteins are recruited to the intracellular tails of many TNFRs in response to receptor trimerization.

The distinguishing features of appropriate transmembrane polypeptides comprise the ability to be expressed at the surface of an immune cell, in particular lymphocyte cells or Natural killer (NK) cells, and to interact together for directing cellular response of immune cell against a predefined target cell. The different transmembrane polypeptides of the CAR of the present invention comprising an extracellular ligand-biding domain and/or a signal transducing domain interact together to take part in signal transduction following the binding with a target ligand and induce an immune response. The transmembrane domain can be derived either from a natural or from a synthetic source. The transmembrane domain can be derived from any membrane-bound or transmembrane protein.

The term “a part of” can refer to any subset of the molecule, that is a shorter peptide. Alternatively, amino acid sequence functional variants of the polypeptide can be prepared by mutations in the DNA which encodes the polypeptide. Such variants or functional variants include, for example, deletions from, or insertions or substitutions of, residues within the amino acid sequence. Any combination of deletion, insertion, and substitution may also be made to arrive at the final construct, provided that the final construct possesses the desired activity, especially to exhibit a specific anti-target cellular immune activity. The functionality of the CAR of the invention within a host cell is detectable in an assay suitable for demonstrating the signaling potential of said CAR upon binding of a particular target. Such assays are available to the skilled person in the art. For example, this assay allows the detection of a signaling pathway, triggered upon binding of the target, such as an assay involving measurement of the increase of calcium ion release, intracellular tyrosine phosphorylation, inositol phosphate turnover, or interleukin (IL) 2, interferon γ, GM-CSF, IL-3, IL-4 production thus effected.

Cells that Express a CAR

Embodiments of the invention include cells that express a CAR (i.e, CARTS). The cell can be of any kind, including an immune cell capable of expressing the CAR for cancer therapy or a cell, such as a bacterial cell, that harbors an expression vector that encodes the CAR. As used herein, the terms “cell,” “cell line,” and “cell culture” can be used interchangeably. All of these terms also include their progeny, which is any and all subsequent generations. It is understood that all progeny doesn't need to be identical, such as due to deliberate or inadvertent mutations. In the context of expressing a heterologous nucleic acid sequence, “host cell” can refer to a eukaryotic cell that is capable of replicating a vector and/or expressing a heterologous gene encoded by a vector. A host cell can, and has been, used as a recipient for vectors. A host cell can be “transfected” or “transformed,” which can refer to a process by which exogenous nucleic acid is transferred or introduced into the host cell. A transformed cell includes the primary subject cell and its progeny. As used herein, the terms “engineered” and “recombinant” cells or host cells can refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, including a cytotoxic T cell (also known as TC, Cytotoxic T Lymphocyte, CTL, T-Killer cell, cytolytic T cell, CD8+ T-cells or killer T cell); NK cells and NKT cells are also encompassed in the invention.

Vectors can employ control sequences that allow it to be replicated and/or expressed in both prokaryotic and eukaryotic cells. One of skill in the art would understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one can wish to be able to kill the modified CTLs, where one wishes to terminate the treatment, the cells become neoplastic, in research where the absence of the cells after their presence is of interest, or other event. For this purpose, one can provide for the expression of certain gene products in which one can kill the modified cells under controlled conditions, such as inducible suicide genes.

### Armed CARTS

The invention further includes CARTS that are modified to secrete one or more polypeptides. Armed CARTS have the advantage of simultaneously secreting a polypeptide at the targeted site, e.g. tumor site. The polypeptide can be for example be an antibody or cytokine. For example, the antibody is specific for ALK, such as antibodies and fragments described herein. In other embodiments, the secreted antibody can be an antibody specific for CAIX, GITR, PD-L2, PD-1, or CCR4 (See, for example, sequences described in PCT Publication No. WO2016/100985, the application which is incorporated by reference in its entirety).

Armed CART can be constructed by including a nucleic acid encoding the secreted polypeptide of interest after the intracellular signaling domain. In embodiments, there is an internal ribosome entry site, (IRES), positioned between the intracellular signaling domain and the polypeptide of interest. One skilled in the art can appreciate that more than one polypeptide can be expressed by employing multiple IRES sequences in tandem.

In embodiments, CART cells can be maintained with the use of cytokines such as, for example, IL-2, IL-4, IL-7, IL-9, IL-15 and IL-21.

Cytokines sharing the γc receptor, like IL-2, IL-4, IL-7, IL-9, IL-15 and IL-21 are important for the development and maintenance of memory T cells. Among them, IL-21 promote a less differentiated phenotype, associated with an enrichment of tumor-specific CD8 T cells, with increased anti-tumor effect in a mouse melanoma model when compared to IL-2 or IL-15.

In certain embodiments, CART cells are maintained with IL-21.

Introduction of Constructs into CTLs

Expression vectors that encode the CARs can be introduced as one or more DNA molecules or constructs, where there may be at least one marker that will allow for selection of host cells that contain the construct(s).

The constructs can be prepared in conventional ways, where the genes and regulatory regions may be isolated, as appropriate, ligated, cloned in an appropriate cloning host, analyzed by restriction or sequencing, or other convenient means. Using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs can be integrated and packaged into non-replicating, defective viral genomes like Adenovirus, Adeno-associated virus (AAV), or Herpes simplex virus (HSV) or others, including retroviral vectors or lentiviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In embodiments, one may have a target site for homologous recombination, where it is desired that a construct be integrated at a particular locus. For example, one can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs can be introduced as a single DNA molecule encoding at least the CAR and optionally another gene, or different DNA molecules having one or more genes. Other genes include genes that encode therapeutic molecules or suicide genes, for example. The constructs can be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that can be used to prepare stocks of construct DNAs and for carrying out transfections are well known in the art, and many are commercially available.

Methods of Use of Cells that Express a CAR

The cells described herein can be used for treating a cancer, or other cell proliferation-related diseases or disorders. Such diseases or disorders include but are not limited to, e.g., those diseases or disorders associated with aberrant expression of ALK. In embodiments, said isolated cell according to the invention can be used in the manufacture of a medicament for treatment a cancer, or other cell proliferation-related diseases or disorders. Such diseases or disorders include but are not limited to, e.g., those diseases or disorders associated with aberrant expression of ALK.

Embodiments described herein rely on methods for treating patients in need thereof, said method comprising at least one of the following steps: (a) providing a chimeric antigen receptor cells according to the invention and (b) administrating the cells to said patient.

Said treatment can be ameliorating, curative or prophylactic. It can be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

The invention is particularly suited for allogenic immunotherapy, insofar as it enables the transformation of T-cells, typically obtained from donors, into non-alloreactive cells. This may be done under standard protocols and reproduced as many times as needed. The resulted modified T cells can be pooled and administrated to one or several patients, being made available as an “off the shelf” therapeutic product.

Cancers that can be treated using the antibody or CAR compositions described herein include tumors that are not vascularized, or not yet substantially vascularized, as well as vascularized tumors. The cancers can comprise nonsolid tumors (such as hematological tumors, for example, leukemias and lymphomas) or can comprise solid tumors. Types of cancers to be treated with the antibodies and CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. For example, cancers of which a checkpoint blockade is a standard therapy for multiple malignancies (referred to herein as “Checkpoint Blockade Cancers”) can be treated with the antibody and/or CAR compositions described herein. Checkpoint Blockade Cancers include, but are not limited to, melanoma, non-small-cell lung cancer (NSCLC), small cell lung cancer (SCLC), renal cell carcinoma (RCC), chronic lymphocytic leukemia (CLL; such as B cell CLL or T cell CLL), classical Hodgkin lymphoma (cHL), head and neck squamous cell carcinoma (HNSCC), colorectal cancer (CRC), gastric cancer, hepatocellular carcinoma (HCC), primary mediastinal large B-cell lymphoma (PMLBCL), bladder cancer, urothelial cancer, endometrial cancer, cervical cancer, breast cancer (e.g., triple negative breast cancer), Merkel cell carcinoma (MCC), and microsatellite instability high (MSI-H) or DNA mismatch repair deficient (dMMR) adult and pediatric solid tumors. The treatments described herein can also include other cancers that are under investigation for checkpoint blockade therapies. Without wishing to be bound by theory, RCC and B-CLL mouse models can be used for treatment with CAR T factories, which are models correlated to the human disease.

For example, treatment can be antibody and/or CAR-T treatment in combination with one or more therapies against cancer selected from the group of antibodies therapy, chemotherapy, cytokines therapy, dendritic cell therapy, gene therapy, hormone therapy, laser light therapy and radiation therapy.

According to an embodiment of the invention, said treatment can be administrated into patients undergoing an immunosuppressive treatment. Indeed, the invention can rely on cells or population of cells, which have been made resistant to at least one immunosuppressive agent due to the inactivation of a gene encoding a receptor for such immunosuppressive agent. In aspects, the immunosuppressive treatment should help the selection and expansion of the T-cells according to the invention within the patient.

In an embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAM PATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery. Said modified cells obtained by any one of the methods described here can be used in a particular aspect of the invention for treating patients in need thereof against Host versus Graft (HvG) rejection and Graft versus Host Disease (GvHD); therefore in the scope of the present invention is a method of treating patients in need thereof against Host versus Graft (HvG) rejection and Graft versus Host Disease (GvHD) comprising treating said patient by administering to said patient an effective amount of modified cells comprising inactivated TCR alpha and/or TCR beta genes.

### Administration of Cells

The invention is suited for allogenic immunotherapy, insofar as it enables the transformation of T-cells, typically obtained from donors, into non-alloreactive cells. This can be done under standard protocols and reproduced as many times as needed. The resulted modified T cells can be pooled and administrated to one or several patients, being made available as an “off the shelf” therapeutic product.

Depending upon the nature of the cells, the cells can be introduced into a host organism, e.g. a mammal, in a wide variety of ways. The cells can be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells can be applied as a dispersion, generally being injected at or near the site of interest. The cells may be in a physiologically-acceptable medium.

In embodiments, the cells are encapsulated to inhibit immune recognition and placed at the site of the tumor.

The cells can be administered as desired. Depending upon the response desired, the manner of administration, the life of the cells, the number of cells present, various protocols can be employed. The number of administrations will depend upon the factors described above at least in part.

The administration of the cells or population of cells according to the invention can be carried out in any convenient manner, including by aerosol inhalation, injection, ingestion, transfusion, implantation or transplantation. The compositions described herein can be administered to a patient subcutaneously, intradermaly, intratumorally, intranodally, intramedullary, intramuscularly, by intravenous or intralymphatic injection, or intraperitoneally. In an embodiment, the cell compositions of the invention are administered by intravenous injection.

The administration of the cells or population of cells can consist of the administration of 10⁴ to 10⁹ cells per kg body weight, such as 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells can be obtained from any source, such as a blood bank or a donor. While individual needs vary, determination of optimal ranges of effective amounts of a given cell type for a particular disease or conditions within the skill of the art. An effective amount can refer to an amount which provides a therapeutic or prophylactic benefit. The dosage administrated will be dependent upon the age, health and weight of the recipient, kind of concurrent treatment, if any, frequency of treatment and the nature of the effect desired.

It should be appreciated that the system is subject to many variables, such as the cellular response to the ligand, the efficiency of expression and, as appropriate, the level of secretion, the activity of the expression product, the particular need of the patient, which can vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

### Nucleic Acid-Based Expression Systems

Monoclonal antibodies and CARs of the present invention can be expressed from an expression vector. Recombinant techniques to generate such expression vectors are well known in the art.

The term “vector” can refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” can refer to any type of genetic construct comprising a nucleic acid coding for an RNA capable of being transcribed. In cases, RNA molecules are then translated into a protein, polypeptide, or peptide. In other cases, these sequences are not translated, for example, in the production of antisense molecules or ribozymes. Expression vectors can contain a variety of “control sequences,” which can refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described herein.

A “promoter” can refer to a control sequence that is a region of a nucleic acid sequence at which initiation and rate of transcription are controlled. It can contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter can comprise a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. These can be located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which can refer to a cis-acting regulatory sequence involved in the transcriptional activation of a nucleic acid sequence.

A promoter can be one naturally associated with a nucleic acid sequence, as may be obtained by isolating the 5 prime′ non-coding sequences located upstream of the coding segment and/or exon. Such a promoter can be referred to as “endogenous.” Similarly, an enhancer can be one naturally associated with a nucleic acid sequence, located either downstream or upstream of that sequence. Alternatively, advantages will be gained by positioning the coding nucleic acid segment under the control of a recombinant or heterologous promoter, which can refer to a promoter that is not normally associated with a nucleic acid sequence in its natural environment. A recombinant or heterologous enhancer can also refer to an enhancer not normally associated with a nucleic acid sequence in its natural environment. Such promoters or enhancers can include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed can be constitutive, tissue-specific, inducible, and/or useful under the appropriate conditions to direct high level expression of the introduced DNA segment, such as is advantageous in the large-scale production of recombinant proteins and/or peptides. The promoter can be heterologous or endogenous.

Additionally, any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art.

A specific initiation signal also can be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In embodiments of the invention, the use of internal ribosome entry sites (IRES) elements are used to create multigene, or polycistronic, messages, and these can be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” can refer to catalytic cleavage of a nucleic acid molecule with an enzyme that functions only at specific locations in a nucleic acid molecule. Many of these restriction enzymes are commercially available. Use of such enzymes is widely understood by those of skill in the art. A vector can be linearized or fragmented using a restriction enzyme that cuts within the MCS to enable exogenous sequences to be ligated to the vector. “Ligation” can refer to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers can also be employed.

In embodiments, a plasmid vector can be used to transform a host cell. Plasmid vectors containing replicon and control sequences which are derived from species compatible with the host cell can be used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™ 11 can be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors can be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the invention can be a viral vector that encodes one or more monoclonal antibodies or CARs of the invention. Non-limiting examples of virus vectors that may be used to deliver a nucleic acid of the present invention are described herein.

A method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

The nucleic acid can be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; Mclaughlin et al., 1988). Details concerning the generation and use of rAAV vectors are described in U.S. Pat. Nos. 5,139,941 and 4,797,368, each incorporated herein by reference.

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the desired sequence) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors can infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus can infect a non-dividing cell wherein a suitable host cell is transfected with two or more vectors carrying the packaging functions, namely gag, pol and env, as well as rev and tat is described in U.S. Pat. No. 5,994,136, incorporated herein by reference. One can target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

Other viral vectors can be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus can be employed. They offer several attractive features for various mammalian cells (Friedmann, 1989; Ridgeway, 1988; Baichwal and Sugden, 1986; Coupar et al., 1988; Horwich et al., 1990).

In embodiments, a nucleic acid to be delivered can be housed within an infective virus that has been engineered to express a specific binding ligand. The virus particle will thus bind specifically to the cognate receptors of the target cell and deliver the contents to the cell. An approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

Methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

### Ex Vivo Transformation

Methods for transfecting eukaryotic cells and tissues removed from an organism in an ex vivo setting are known to those of skill in the art. Thus, it is contemplated that cells or tissues can be removed and transfected ex vivo using nucleic acids of the invention. In aspects, the transplanted cells or tissues can be placed into an organism. In embodiments, a nucleic acid is expressed in the transplanted cells.

### Kits of the Invention

Any of the compositions described herein can be comprised in a kit.

Some components of the kits can be packaged either in aqueous media or in lyophilized form. The container means of the kits can include at least one vial, test tube, flask, bottle, syringe or other container means, into which a component can be placed, and suitably aliquoted. Where there is more than one component in the kit, the kit also can contain a second, third or other additional container into which the additional components can be separately placed. However, various combinations of components can be comprised in a vial. The kits of the invention also can include a means for containing the components in close confinement for commercial sale. Such containers can include injection or blow molded plastic containers into which the desired vials are retained.

When the components of the kit are provided in one and/or more liquid solutions, the liquid solution is an aqueous solution, with a sterile aqueous solution being useful. In some cases, the container means can itself be a syringe, pipette, and/or other such like apparatus, from which the formulation can be applied to an infected area of the body, injected into an animal, and/or even applied to and/or mixed with the other components of the kit.

However, the components of the kit can be provided as dried powder(s). When reagents and/or components are provided as a dry powder, the powder can be reconstituted by the addition of a suitable solvent. It is envisioned that the solvent can also be provided in another container means. The kits can also comprise a second container means for containing a sterile, pharmaceutically acceptable buffer and/or another diluent.

In embodiments of the invention, cells that are to be used for cell therapy are provided in a kit, and in some cases the cells can be the sole component of the kit. The kit can comprise reagents and materials to make the desired cell. In embodiments, the reagents and materials include primers for amplifying desired sequences, nucleotides, suitable buffers or buffer reagents, salt, and so forth, and in some cases the reagents include vectors and/or DNA that encodes a CAR as described herein and/or regulatory elements therefor.

In embodiments, there are one or more apparatuses in the kit suitable for extracting one or more samples from an individual. The apparatus can be a syringe, scalpel, and so forth.

In embodiments of the invention, the kit, in addition to cell therapy embodiments, also includes a second cancer therapy, such as chemotherapy, hormone therapy, and/or immunotherapy, for example. The kit(s) can be tailored to a particular cancer for an individual and comprise respective second cancer therapies for the individual.

### Assays

Aspects of the invention comprise assays, for example assays that detect the presence of and/or measure levels of ALK or a fragment thereof.

Embodiments of the invention comprise measuring or detecting ALK using assays known to the art. Non-limiting examples of assays include an immunoassay, a colorimetric assay, fluorimetric assay or a combination thereof. Non-limiting examples of immunoassays comprise a western blot assay, an enzyme-linked immunosorbent assay (ELISA), immunoprecipitation or a combination thereof. For example, a biological sample collected from a subject can be incubated together with an anti-ALK antibody according to the invention, and the binding of the antibody to the biomarker in the sample is detected or measured. In embodiments, the antibody or fragment thereof can be specific for ALK. The antibody can be a polyclonal antibody or a monoclonal antibody. The antibody or fragment thereof can be attached to a molecule that is capable of identification, visualization, or localization using known methods. Detectable labels include but are not limited to radioisotopic labels, enzyme labels, non-radioactive isotopic labels, fluorescent labels, toxin labels, affinity labels, and chemiluminescent labels.

In embodiments, the assays can be provided in a multi-well format, such as a 6-, 12-, 24-, 48, or 96-well plate.

The anti-ALK antibodies can be used diagnostically to, for example, detect cancer or a cell-proliferative disease, detect the recurrence of cancer or a cell-proliferative disease, monitor the development or progression of cancer as part of a clinical testing procedure to, e.g., determine the efficacy of a given treatment and/or prevention regimen.

“Changed as compared to a control” sample or subject is understood as having a level of the analyte or diagnostic or therapeutic indicator (e.g., marker such as ALK) to be detected at a level that is statistically different than a sample from a normal, untreated, or abnormal state control sample. Determination of statistical significance is within the ability of those skilled in the art, e.g., the number of standard deviations from the mean that constitute a positive or negative result and the statistical analyses to arrive at these intervals.

If a subject is diagnosed with a cell-proliferative disease, such as cancer, embodiments of the invention comprise treating the subject. For example, treating the subject can comprise administering to the subject an effective anti-cancer agent, including those described herein.

The term “threshold”, for example, a threshold indicative of a cell-proliferative disease, such as cancer, can refer to a value derived from a plurality of biological samples, such as cancer samples or donor blood samples, for a biomarker, such as ALK protein levels, above which threshold is associated with an increased likelihood of having and/or developing a cell proliferative disease, such as cancer.

In aspects for diagnostic purposes, the anti-ALK antibody of the invention can be linked to a detectable moiety, for example, so as to provide a method for detecting a cancer cell in a subject at risk of or suffering from a cancer.

The detectable moieties can be conjugated directly to the antibodies or fragments, or indirectly by using, for example, a fluorescent secondary antibody. Direct conjugation can be accomplished by standard chemical coupling of, for example, a fluorophore to the antibody or antibody fragment, or through genetic engineering. Chimeras, or fusion proteins can be constructed which contain an antibody or antibody fragment coupled to a fluorescent or bioluminescent protein. For example, Casadei, et al, (Proc Natl Acad Sci USA. 1990 March; 87(6):2047-51) describe a method of making a vector construct capable of expressing a fusion protein of aequorin and an antibody gene in mammalian cells.

The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject (such as a biopsy), as well as tissues, cells and fluids present within a subject. That is, the detection method of the invention can be used to detect cells that express ALK in a biological sample in vitro as well as in vivo. For example, in vitro techniques for detection of ALK include enzyme linked immunosorbent assays (ELISAs), Western blots, immunoprecipitations, and immunofluorescence. Furthermore, in vivo techniques for detection of ALK include introducing into a subject a labeled anti-ALK antibody. For example, the antibody can be labeled with a radioactive marker whose presence and location in a subject can be detected by standard imaging techniques.

In the case of “targeted” conjugates, that is, conjugates which contain a targeting moiety—a molecule or feature designed to localize the conjugate within a subject or animal at a particular site or sites, localization can refer to a state when an equilibrium between bound, “localized”, and unbound, “free” entities within a subject has been essentially achieved. The rate at which such equilibrium is achieved depends upon the route of administration. For example, a conjugate administered by intravenous injection can achieve localization within minutes of injection. On the other hand, a conjugate administered orally can take hours to achieve localization. Alternatively, localization can simply refer to the location of the entity within the subject or animal at selected time periods after the entity is administered. By way of another example, localization is achieved when a moiety becomes distributed following administration.

It is understood that a reasonable estimate of the time to achieve localization can be made by one skilled in the art. Furthermore, the state of localization as a function of time can be followed by imaging the detectable moiety (e.g., a light-emitting conjugate) according to the methods of the invention, such as with a photodetector device. The “photodetector device” used should have a high enough sensitivity to enable the imaging of faint light from within a mammal in a reasonable amount of time, and to use the signal from such a device to construct an image.

In cases where it is possible to use light-generating moieties which are extremely bright, and/or to detect light-generating fusion proteins localized near the surface of the subject or animal being imaged, a pair of “night-vision” goggles or a standard high-sensitivity video camera, such as a Silicon Intensified Tube (SIT) camera (e.g., from Hammamatsu Photonic Systems, Bridgewater, N.J.), can be used. More typically, however, a more sensitive method of light detection is required.

In extremely low light levels, the photon flux per unit area becomes so low that the scene being imaged no longer appears continuous. Instead, it is represented by individual photons which are both temporally and spatially distinct form one another. Viewed on a monitor, such an image appears as scintillating points of light, each representing a single detected photon. By accumulating these detected photons in a digital image processor over time, an image can be acquired and constructed. In contrast to conventional cameras where the signal at each image point is assigned an intensity value, in photon counting imaging the amplitude of the signal carries no significance. The objective is to simply detect the presence of a signal (photon) and to count the occurrence of the signal with respect to its position over time.

At least two types of photodetector devices, described below, can detect individual photons and generate a signal which can be analyzed by an image processor. Reduced-Noise Photodetection devices achieve sensitivity by reducing the background noise in the photon detector, as opposed to amplifying the photon signal. Noise is reduced primarily by cooling the detector array. The devices include charge coupled device (CCD) cameras that can be to as “backthinned”, cooled CCD cameras. In the more sensitive instruments, the cooling is achieved using, for example, liquid nitrogen, which brings the temperature of the CCD array to approximately −120° C. “Backthinned” can refer to an ultra-thin backplate that reduces the path length that a photon follows to be detected, thereby increasing the quantum efficiency. A particularly sensitive backthinned cryogenic CCD camera is the “TECH 512”, a series 200 camera available from Photometries, Ltd. (Tucson, Ariz.).

“Photon amplification devices” amplify photons before they hit the detection screen. This class includes CCD cameras with intensifiers, such as microchannel intensifiers. A microchannel intensifier typically contains a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera.

Even greater sensitivity can be achieved by placing intensifying microchannel arrays in series, so that electrons generated in the first stage in turn result in an amplified signal of electrons at the second stage. Increases in sensitivity, however, are achieved at the expense of spatial resolution, which decreases with each additional stage of amplification. An exemplary microchannel intensifier-based single-photon detection device is the C2400 series, available from Hamamatsu.

Image processors process signals generated by photodetector devices which count photons in order to construct an image which can be, for example, displayed on a monitor or printed on a video printer. Such image processors are typically sold as part of systems which include the sensitive photon-counting cameras described above, and accordingly, are available from the same sources. The image processors are usually connected to a personal computer, such as an IBM-compatible PC or an Apple Macintosh (Apple Computer, Cupertino, Calif), which may or may not be included as part of a purchased imaging system. Once the images are in the form of digital files, they can be manipulated by a variety of image processing programs (such as “ADOBE PHOTOSHOP”, Adobe Systems, Adobe Systems, Mt. View, Calif.) and printed.

In an embodiment, the biological sample contains protein molecules from the test subject. For example, a biological sample can be a peripheral blood leukocyte sample isolated by conventional means from a subject.

The invention also encompasses kits for detecting the presence of ALK or an ALK-expressing cell in a biological sample. For example, the kit can comprise: a labeled compound or agent capable of detecting a cancer or tumor cell (e.g., an anti-ALK monoclonal antibody) in a biological sample; means for determining the amount of ALK in the sample; and means for comparing the amount of ALK in the sample with a standard. The standard is, in some embodiments, a non-cancer cell or cell extract thereof. The compound or agent can be packaged in a suitable container. The kit can further comprise instructions for using the kit to detect cancer in a sample.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

The anaplastic lymphoma kinase (ALK) receptor is a therapeutic target in neuroblastoma, a tumor of the peripheral sympathetic nervous system. It is a cell surface receptor that is expressed in more than 90% of primary neuroblastoma tumors and mutationally activated in 10%, the latter being sensitive to small molecule inhibitors. The extracellular domain of ALK is cleaved (shed) and can be recovered from cell culture media and/or sera from patients with neuroblastoma. ALK has a restricted expression pattern in the adult and high expression is seen mainly in tumors and not normal tissues. Thus, ALK is also an antigenic target for immune-directed therapy in neuroblastoma. The full length ALK receptor is overexpressed also in other malignancies including rhabdomyosarcoma, glioma, thyroid cancer. ALK is also aberrant in other cancers such as non-small cell lung cancer, inflammatory myofibroblastic tumors and anaplastic large cell lymphoma where it forms part of an oncogenic fusion protein due to translocation of the intracytoplasmic portion of ALK to another protein.

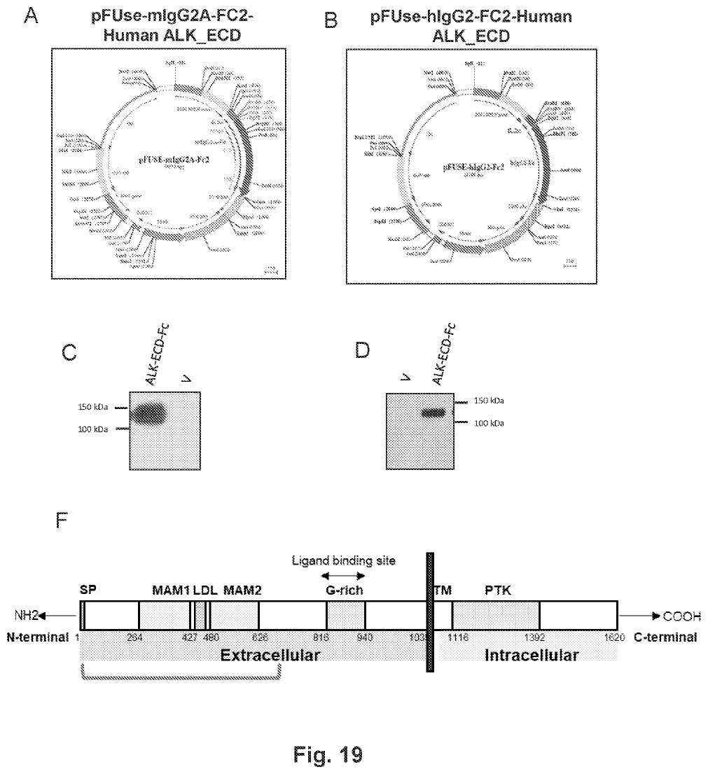

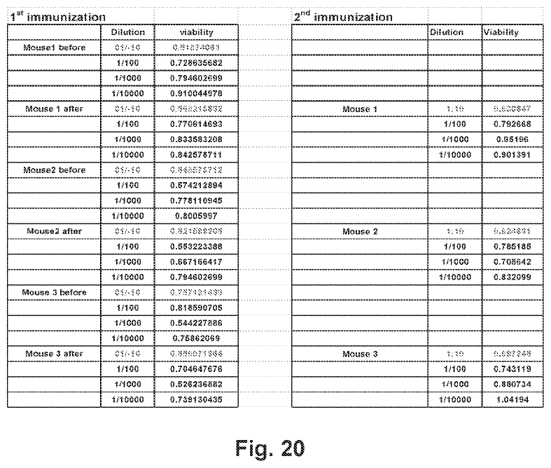

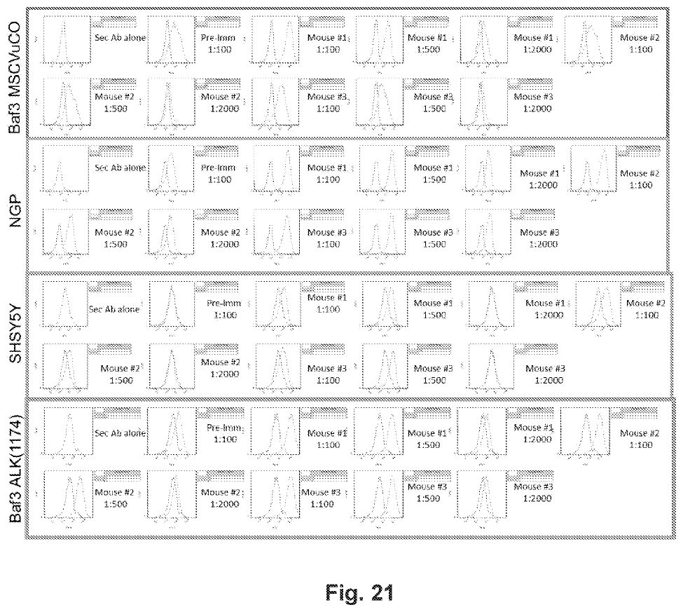

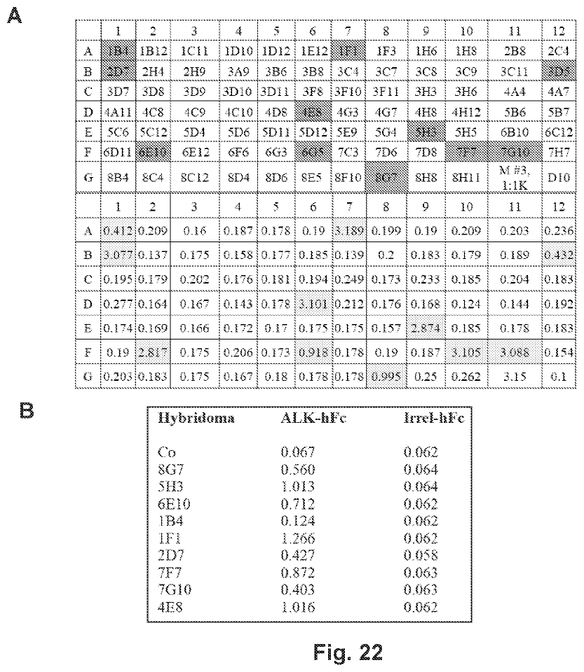

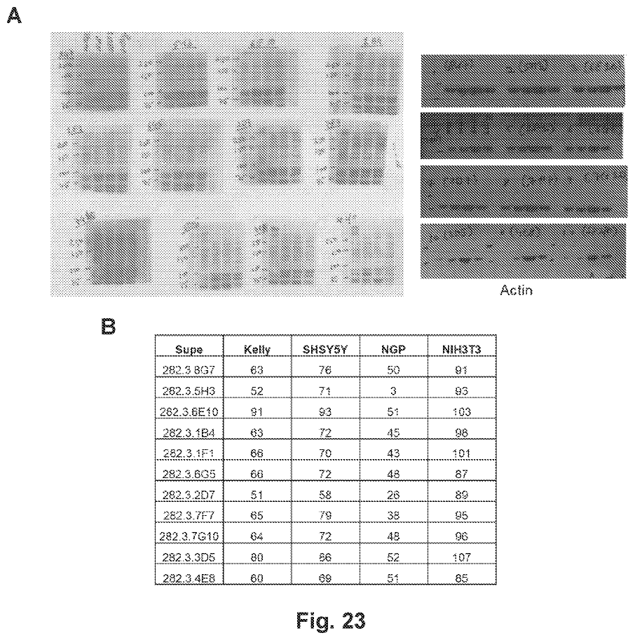

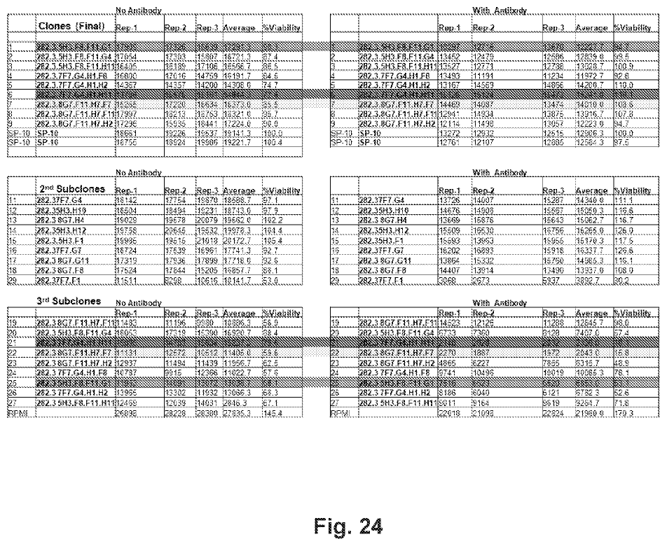

We have generated murine monoclonal antibodies that recognize the N-terminal portion of the human ALK protein with high specificity and selectivity. We used the human ALK extracellular domain (19-682 aa) fused to the mouse Fc gene as the immunogen to raise antibodies (FIG. 19). Because of the similarity between mouse and human ALK protein sequences, we used transgenic ALK-null mice which are immune competent. Screening of sera was performed by ELISA, western blotting and flow cytometry (FCM) on cell lines expressing both wild-type and mutated ALK (FIG. 20 and FIG. 21). Based on these assays, we prioritized one mouse for hybridoma fusion with two others as back-up. Of 800+ hybridoma supernatants screened by ELISA, FCM, and functional assays in neuroblastoma cell lines expressing ALK, 6 parentals were initially selected for further analysis on the basis of specificity of antigen binding (FIG. 22 and FIG. 23). These were subcloned by limiting dilution and from these clones, we identified 3 individual clones, each producing a monoclonal antibody (8G7, 5H3 and 7F7) with high ALK specificity (FIG. 24).

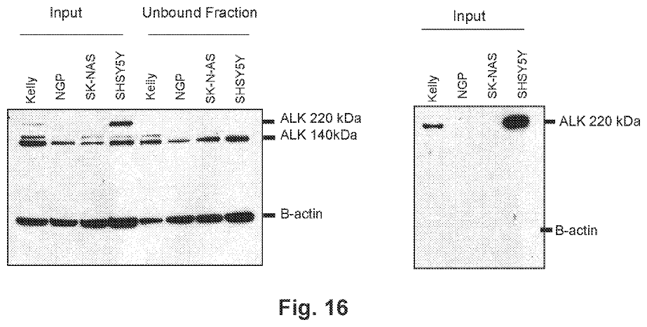

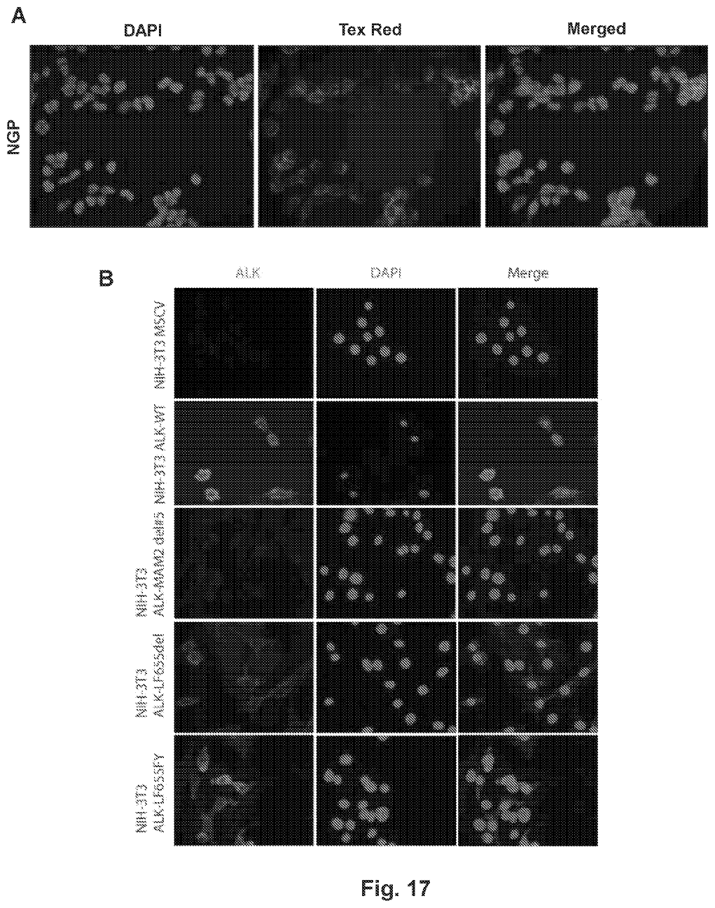

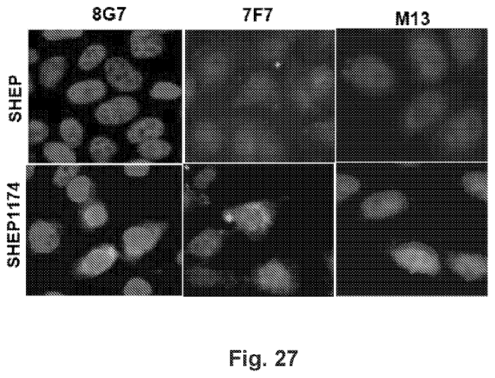

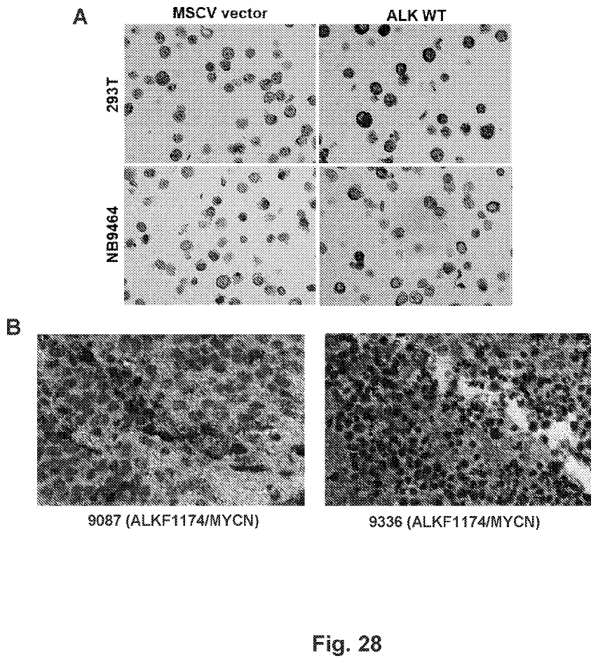

In embodiments, the monoclonal antibodies described herein, such as 8G7, 5H3, and 7F7, recognize ALK in immunoblotting assays and can immunoprecipitate ALK (FIG. 16). Further, the monoclonal antibodies described herein, such as 8G7, also recognize ALK at the cell surface by immunofluorescence, immunocytochemistry and immunohistochemistry (FIG. 17, FIG. 27 and FIG. 28).

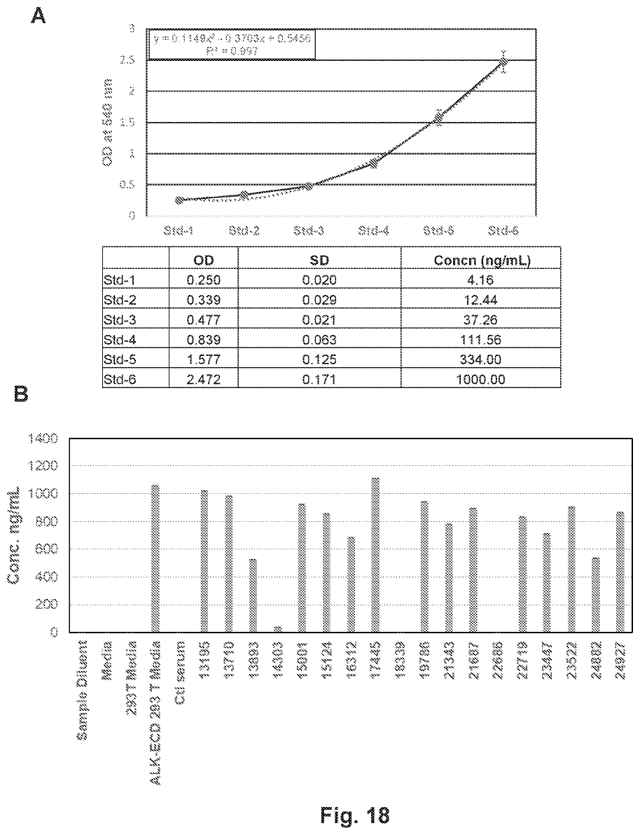

Also, the monoclonal antibodies described herein, such as 8G7, 5H3, and 7F7, recognize the circulating ALK extracellular fragment in the sera of patients with ALK-expressing neuroblastoma (FIG. 18).

We developed a sandwich ELISA technique where two ALK antibodies (for example, 5H3 or 7F7) can be used to capture the ALK extracellular fragment (antigen), and another antibody, such as the 8G7 antibody, can be used to detect the captured ALK extracellular fragment-5H3 or 7F7 complex. The different epitope binding sites of these antibodies on the ALK extracellular fragment protein allow for the development of the sandwich ELISA technique for the detection of the cleaved or shed ALK extracellular fragment in patient serum samples (FIG. 18). Verification, such as in a larger number of samples, will indicate that the antibodies can be used to monitor disease response, and/or progression of neuroblastoma and other cancers in which the full-length ALK protein is expressed and cleaved in the extracellular domain.

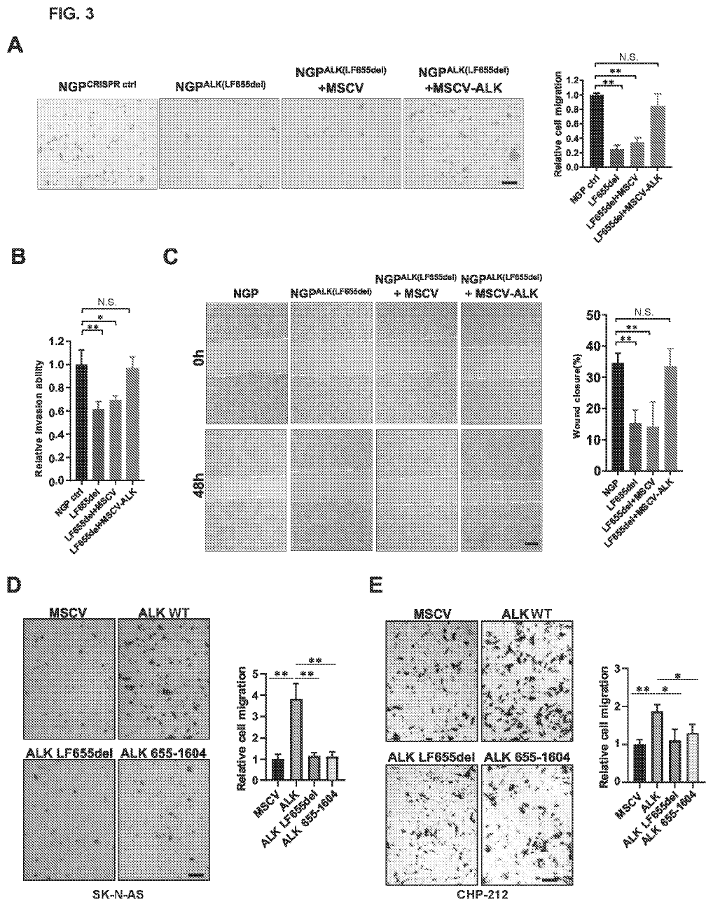

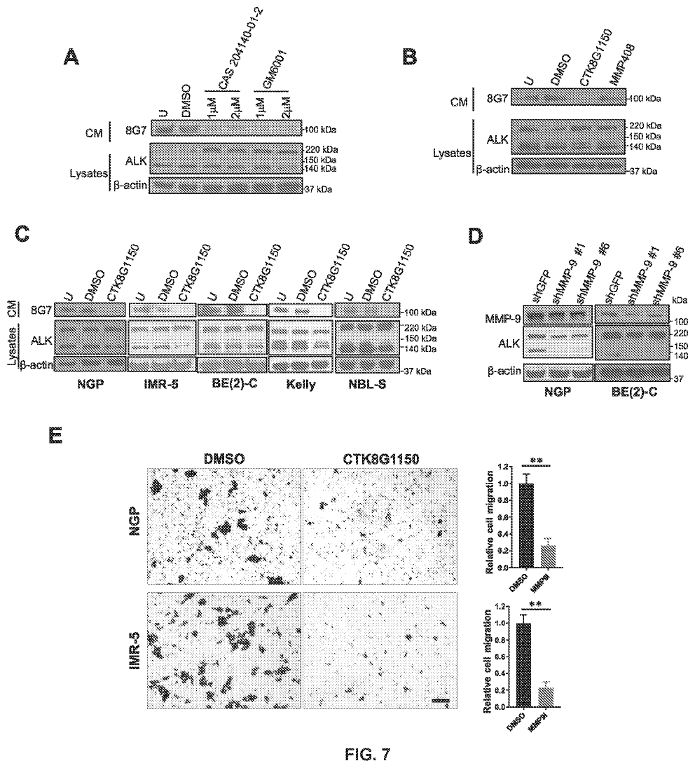

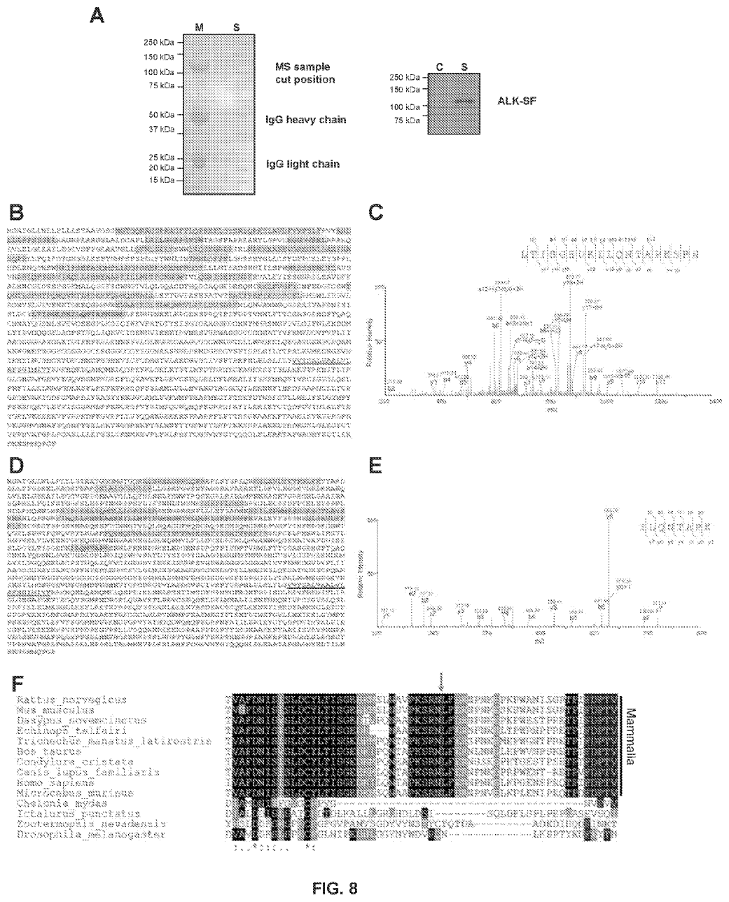

Further, we have used monoclonal antibodies described herein, such as the 8G7 antibody, to identify the extracellular domain cleavage site of the ALK receptor (FIG. 8). This led to further studies where we determined that ECD shedding is an aspect of the ALK receptor, and that it leads to migration and invasion of cells as well as an epithelial-to-mesenchymal phenotype (FIG. 3). Further, we also identified that the matrix-metalloprotease (MMP)-9 induces ALK ECD shedding and that MMP-9 inhibitors can prevent such shedding, thererby indicating a therapeutic option for increasing the availability of the cell surface expression of ALK that can be utilized as a tumor-associated antigen (FIG. 7).

Monoclonal antibodies described herein, for example 8G7, are superior to that of other anti-ALK antibodies in their specificity for the N-terminal portion of ALK.

Monoclonal antibodies described herein, for example 8G7, can identify ALK expression on the cell surface. Such uses, therefore, include for research and clinical purposes.

Monoclonal antibodies described herein can be used for biotherapy or immunotherapy as wild-type ALK has all the characteristics of an immune target, such as being overexpressed on the cell surface and has a restricted pattern of expression.

Further, monoclonal antibodies described herein can be useful for detecting ALK as a tumor biomarker or tumor indicator in the sera of patients with ALK-expressing cancers, such as ALK-expressing neuroblastoma. Such indicators can be used for diagnostic methods, and also for the early identification of tumor recurrence.

### Example 2

### Extracellular Domain Shedding of the ALK Receptor Mediates Neuroblastoma Cell Migration

Mutations of the anaplastic lymphoma kinase (ALK) cell surface receptor tyrosine kinase are the most common directly targetable genetic aberrations in neuroblastoma, affecting approximately 10% of these tumors. However, the wild-type receptor is also overexpressed in the vast majority of neuroblastoma tumors, where it undergoes proteolytic shedding of the extracellular domain (ECD), the functional significance of which is unclear. Here we mapped the ALK cleavage site to Asn654-Leu655 and show that ECD shedding is important for neuroblastoma cell invasion and migration. CRISPR-based editing of the ECD cleavage site of the wild-type ALK receptor in neuroblastoma cells resulted in their decreased migration and invasion, a phenotype that was associated with downregulation of an epithelial-to-mesenchymal signature, decreased nuclear localization of β-catenin and reversal upon reintroduction of wild-type ALK. Inhibition of ECD cleavage led to significantly decreased metastasis and longer survival in mouse models of neuroblastoma. Retention of the ECD also potentiated retinoic acid-induced differentiation of neuroblastoma cells. We further show that the ALK ECD is cleaved by matrix metalloproteinase 9, whose inactivation leads to cleavage inhibition and loss of neuroblastoma cell migration. Together, our results indicate roles for ECD cleavage of wild-type ALK in neuroblastoma metastasis.

### Example 3

### Extracellular Domain Shedding of the ALK Receptor Mediates Neuroblastoma Cell Migration

Mutations of the anaplastic lymphoma kinase (ALK) membrane receptor are the targetable genetic aberrations in neuroblastoma (NB). Both wild-type (WT) and mutated ALK are overexpressed in NB, where they undergo proteolytic shedding of the extracellular domain (ECD), the significance of which is unclear. Here we mapped the ECD cleavage site to Asn654-Leu655 and determined that inhibition of WT ALK ECD cleavage results in decreased cell migration and invasion, a phenotype that is associated with downregulation of an epithelial-to-mesenchymal transition (EMT) signature. ALK ECD cleavage inhibition decreases the nuclear localization of β-catenin, leading to its reduced occupancy at target genes with roles in EMT and their decreased expression. Cleavage inhibition also led to decreased metastases and prolonged survival in mouse models of NB. WT ALK ECD shedding is mediated by matrix metalloproteinase-9, whose genetic or pharmacologic inactivation caused cleavage inhibition and decreased NB cell migration. Together, our results indicate a pivotal role for WT ALK ECD cleavage in NB cell migration and indicate approaches to harness this process for therapeutic gain.

Aberrant expression of the anaplastic lymphoma kinase (ALK) receptor is driven by point mutations, amplification or protein fusions and provides a tractable therapeutic target in cancers where these mutant proteins contribute to the malignant phenotype (Hallberg and Palmer, 2016). Neuroblastoma (NB), a tumor of the sympathetic nervous system arising from the neural crest, affords an excellent example, as the identification of activating mutations of the ALK gene have led to clinical trials of ALK inhibitors in this subgroup of patients (Chen et al., 2008; George et al., 2008; Janoueix-Lerosey et al., 2008; Mosse et al., 2008). Such lesions, occurring in approximately 10% of tumors, are located in the intracellular kinase domain of this membrane receptor, leading to its constitutive phosphorylation and activation of downstream signaling pathways that induce abnormal cell proliferation. In addition, the wild-type (WT) ALK receptor, expressed in the developing nervous system where it facilitates apoptosis through caspase-mediated cleavage of its intracytoplasmic segment (Mourali et al., 2006), is overexpressed in almost 90% of NB tumors (Lamant et al., 2000; Passoni et al., 2009). However, the role of WT ALK if any, in tumorigenesis is unclear, although certain observations indicate that it can contribute to the cancer phenotype. First, increased WT ALK expression in NB correlates with a poor patient outcome (De Brouwer et al., 2010; Passoni et al., 2009; Schulte et al., 2011). Second, activation of WT ALK has been linked to the proliferation, migration and invasion of NB cells and downstream signaling in HEK293 and PC12 pheochromocytoma cells (Hasan et al., 2013; Moog-Lutz et al., 2005; Motegi et al., 2004; Souttou et al., 2001). Third, genetic and pharmacologic depletion of WT ALK expression in NB cell lines causes growth arrest and cell death (Di Paolo et al., 2011; Mosse et al., 2008; Passoni et al., 2009). These results indicate that in most NB tumors in which ALK is overexpressed, mechanisms other than DNA alterations lead to its aberrant function and may therefore contribute to the pathophysiology of the tumor.

In NB cell lines and primary tumors, the ALK protein is expressed as a 220 kDa form, representing the heavily glycosylated full-length receptor and as a shorter 140 kDa product, resulting from cleavage or shedding of the N-terminal extracellular domain (ECD) (Moog-Lutz et al., 2005; Osajima-Hakomori et al., 2005). The significance of such ECD shedding is unknown, although structural alterations that remove some or all of the ECD-encoding exons 1-4 and some of which lead to ALK activation (Okubo et al., 2012; Souttou et al., 2001), have been reported in NB cell lines and 2.4% of primary tumors (Brady et al., 2020; Cazes et al., 2013; Fransson et al., 2015; Okubo et al., 2012), indicating a repressor role for this domain. Additionally, culture of NB cells in Schwann cell media abolished ALK ECD cleavage (Degoutin et al., 2009), consistent with the differentiated nature and excellent patient outcome of Schwannian stroma-rich compared with stroma-poor tumors (Shimada et al., 1984). Without wishing to be bound by theory, removal of the ALK ECD contributes to the malignant phenotype of NB cells. To pursue this notion, we undertook studies to map the region of the ALK receptor that is targeted by ECD cleavage, the mechanism(s) responsible for this process as well as the functional consequences of this modification in NB.

### ALK ECD Cleavage Generates a Shed Ectodomain and a Membrane-Bound Fragment

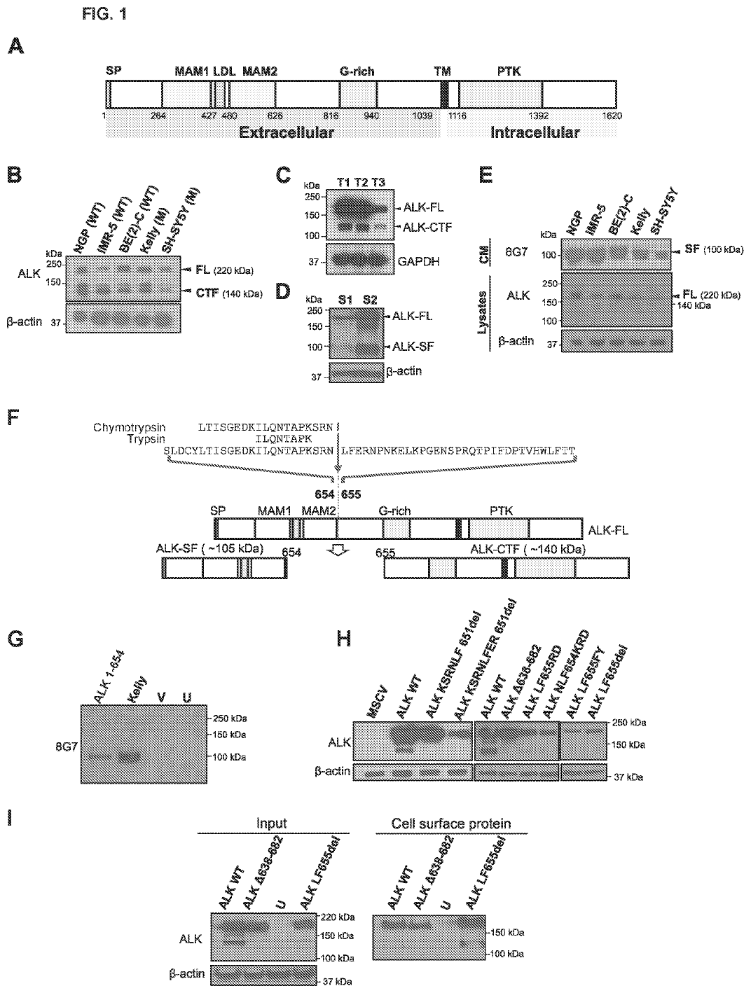

The full-length ALK protein comprises an extracellular, a transmembrane and an intracellular domain (FIG. 1, panel A). The ECD, which contains binding sites for the ALK ligands ALKAL1 and ALKAL2 (previously named FAM150A/B) (Guan et al., 2015) and Augα/β (Reshetnyak et al., 2015), consists of an N-terminal signal peptide, two meprin-A5-protein receptor protein tyrosine phosphatase Mu (MAM) domains separated by a low-density lipoprotein class A motif (LDLa), and a glycine-rich region while the intracellular domain includes a catalytic protein tyrosine kinase domain (Iwahara et al., 1997). To verify the existence of ALK cleavage in NB cells, we first analyzed human NB cell lines using an antibody directed to the C-terminal domain of ALK, which revealed both the 220 kDa full-length receptor and the 140 kDa products (FIG. 1, panel B). The two protein products were identified in all NB cells that expressed ALK, irrespective of ALK mutation status. ALK ECD cleavage was also apparent in patient-derived xenograft (PDX) NB models (FIG. 1, panel C), and consistent with the highest expression of ALK occurring during development (Iwahara et al., 1997; Morris et al., 1994), the two ALK fragments were also observed in neonatal murine brain tissue (FIG. 1, panel D). To ensure that the 140 kDa fragment resulted from proteolytic cleavage of the full-length receptor, we used 8G7, an N-terminal ALK monoclonal antibody that binds to the ECD of human ALK with high specificity, to analyze conditioned media from NB cell lines. An ALK fragment with a molecular mass of approximately 100 kDa was identified in the conditioned media of all five NB cell lines whose lysates were positive for the full-length 220 kDa and the truncated 140 kDa products, confirming ectodomain shedding (FIG. 1, panel E). Consistent with this finding, the 8G7 antibody detected the 220 kDa full-length protein but not the 140 kDa fragment in cell lysates (FIG. 1, panel E). Thus, ECD cleavage or shedding is common to both WT and mutated ALK, is conserved across malignant and non-malignant tissue contexts and is an integral feature of the ALK receptor.

### ECD Cleavage Occurs Between the MAM2 Domain and the Glycine-Rich Region of the ALK Receptor

To identify the cleavage site within the ECD, we used the 8G7 N-terminal antibody to isolate the shed ALK fragment by immunoprecipitation from conditioned media of NGP human NB cells that endogenously express both full-length and cleaved WT ALK (FIG. 1, panel E; FIG. 8, panel A). The purified ALK ECD protein was then enzymatically digested with chymotrypsin and subjected to HPLC-tandem mass spectrometry (LC-MS/MS). Chymotrypsin digestion yielded several peptide fragments whose C-termini corresponded to the YWFL (tyrosine, tryptophan, phenylalanine and leucine) consensus site for chymotrypsin digestion. In two fully independent replicates, another peptide fragment, ending at asparagine 654 (N or Asn654), 636-LTISGEDKILQNTAPKSRN-654 (SEQ ID NO: 53), was also seen (FIG. 1, panel F; FIG. 8, panels B and C). Since this residue is not a typical chymotrypsin digestion site, without wishing to be bound by theory, Asn654 can be the natural ECD cleavage site. To validate that this was the case, we digested the ALK ECD protein with trypsin. Trypsin digestion generated peptides that terminated at the consensus trypsin digestion site [lysine (K) or arginine (R], one of which, 644-ILQNTAPK-651 (SEQ ID NO: 54), was located N-terminal to the putative ALK ECD cleavage site at Asn654 and which corresponds to part of the same peptide fragment that was identified in the chymotrypsin digest (FIG. 1, panel F; FIG. 8, panels D and E). The similar molecular weights of an engineered ALK ECD construct (ALK1-654) expressed in 293T cells that do not endogenously express ALK and the shed ALK fragment (FIG. 1, panel G) validates this interpretation. The putative cleavage site and its consensus sequence are highly conserved across mammalian species (FIG. 8, panel F), indicates an essential role for ECD cleavage in ALK-expressing cells.

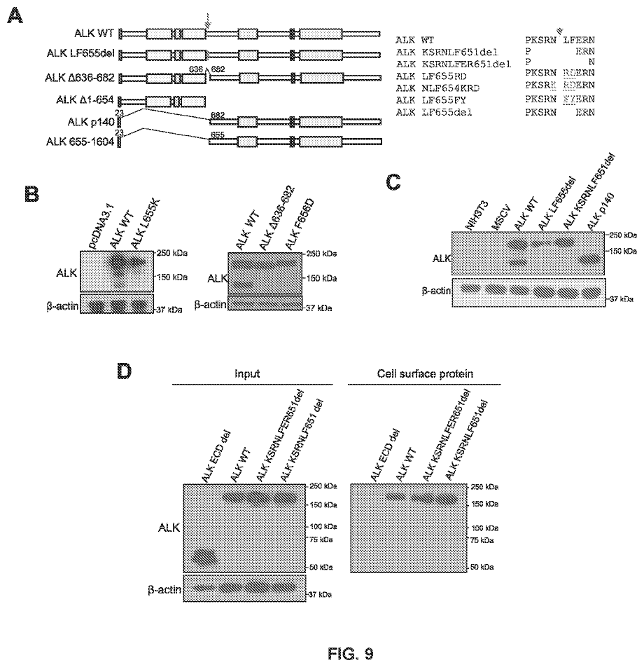

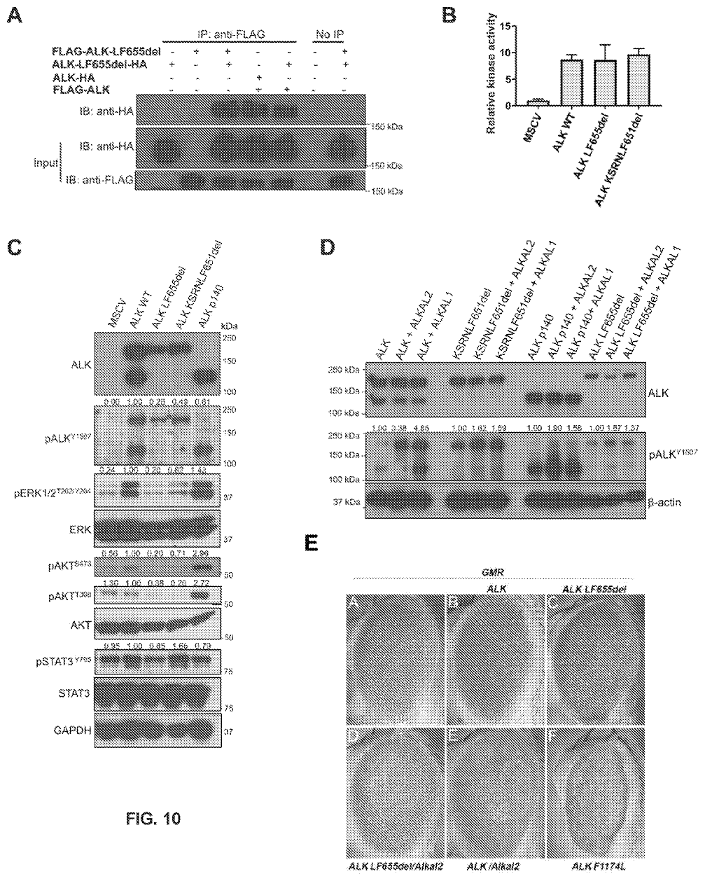

To verify the identity of this cleavage site, we generated several constructs with mutations or deletions at this position (cleavage site mutants, CSMs; FIG. 9, panel A), and examined their cleavage efficiencies when overexpressed in 293T cells (FIG. 1, panel H). As a positive control, we used WT ALK, which upon expression in these cells undergoes cleavage, resulting in the 220 and 140 kDa products; the negative control was the ALK 4638-682 construct, which contains a 45 aa deletion C-terminal to the MAM2 domain and does not undergo cleavage. All the CSM constructs showed different degrees of cleavage inhibition, with deletions or mutations of the amino acids at positions 655 and 656 identified as the minimum changes required for cleavage inhibition (FIG. 1, panel H; FIG. 9, panel B). Similar results were observed in NIH3T3 cells lacking endogenous ALK expression but engineered to express WT and CSMs (FIG. 9, panel C). Using biotinylation assays we confirmed that ALK CSM proteins, especially ALK LF655del, when overexpressed in 293T cells, were presented to the cell membrane thus enabling further functional studies (FIG. 1, panel I; FIG. 9, panel D). Similar to WT ALK, the ALK LF655del CSM was able to homo- and heterodimerize with the WT receptor (FIG. 10, panel A) and exhibited similar kinase activity (FIG. 10, panel B). When overexpressed in NIH3T3 cells, ALK LF655del showed decreased phosphorylation and downstream signaling compared to WT ALK (FIG. 10, panel C). The ALK ligands, ALKAL1 and ALKAL2 (Guan et al., 2015; Reshetnyak et al., 2015), that bind to the glycine-rich region C-terminal to the putative cleavage site also upregulated ALK LF655del phosphorylation, but to a much lesser extent than WT ALK (FIG. 10, panel D). Similar results were observed when the CSMs were tested in the Drosophila eye model (FIG. 10, panel E). Considered together, these results confirm Asn654-Leu655 as the ECD cleavage site of ALK and demonstrate that cleavage inhibition retains the intrinsic properties of the WT receptor, albeit at decreased levels.

### The LF655del ALK Mutation Inhibits ALK Cleavage in NB Cells

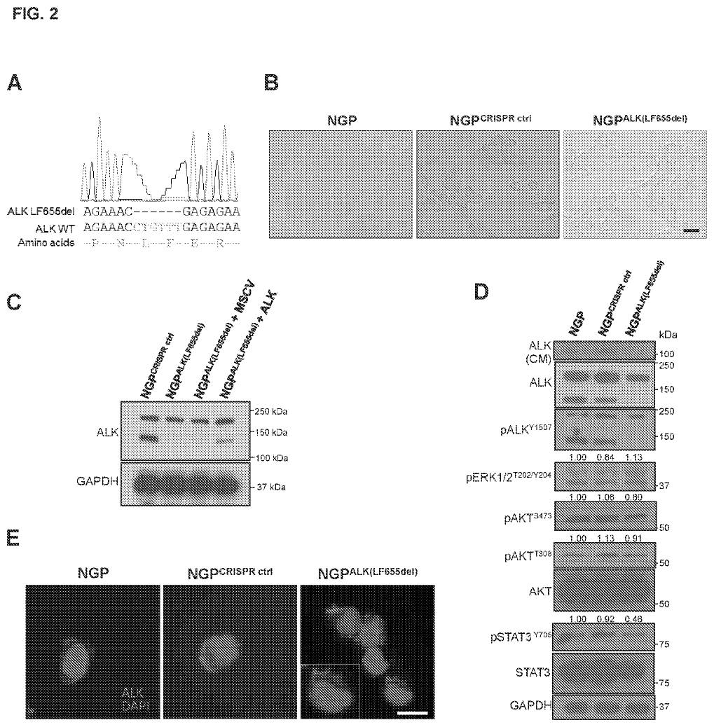

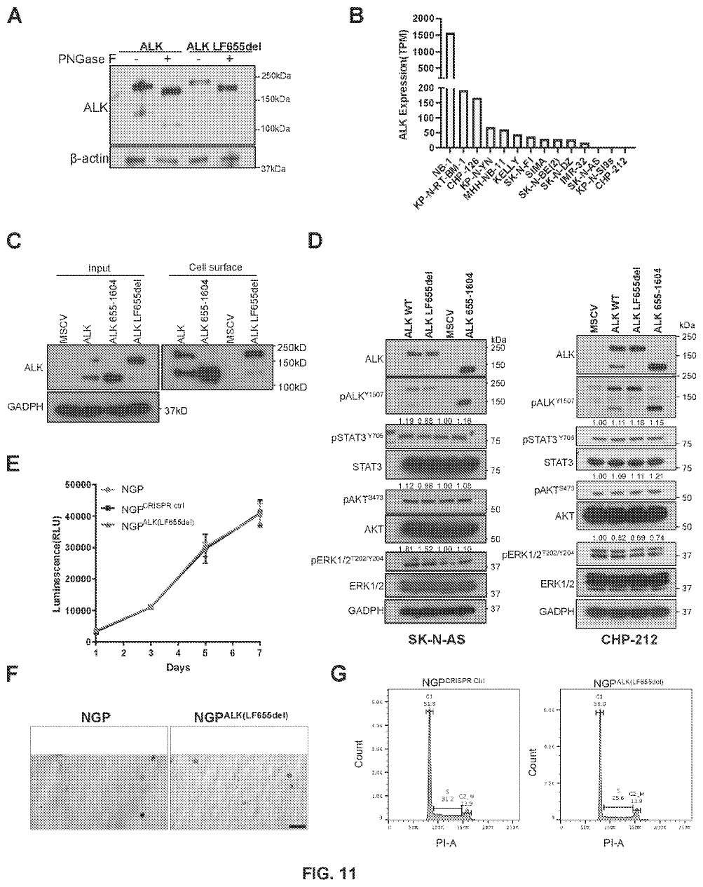

To validate the functional effects of ALK ECD cleavage in the NB cell context, we used CRISPR/Cas9-mediated editing to introduce the LF655del mutation into the endogenous ALK locus in NGP NB cells that overexpress full-length WT ALK that is spontaneously cleaved in its ECD. Single cell selection yielded the NGPALK(LF655del) knock-in clone, in which biallelic editing was confirmed by sequencing (FIG. 2, panel A). NGPALK(LF655del) cells appeared to be morphologically different from NGPCRISPR ctrl (NGP cells expressing a control editing vector) cells and tended to adhere together in clumps (FIG. 2, panel B). NGPALK(LF655del) cells exhibited a predominance of the 220 kDa full-length protein, with the 140 kDa truncated form being absent or present at extremely low levels, an effect that could be rescued by overexpression of WT ALK (FIG. 2, panel C). The shed ALK ECD fragment was absent in conditioned media from these cells confirming inhibition of cleavage (FIG. 2, panel D). Phosphorylation of the 220 full-length band was similar in NGPALK(LF655del) and NGPCRISPR ctrl cells, and unlike NIH3T3 cells in which the ALK LF655del CSM was ectopically expressed, NGPALK(LF655del) cells showed no differences in downstream signaling pathways (FIG. 2, panel D). We also ascertained that the CRISPR-edited ALK receptor was still expressed at the NGP cell membrane through immunofluorescence assays (FIG. 2, panel E) and that it was glycosylated (FIG. 11, panel A). To ensure that these results were not unique to a single NB cell line or the method of cleavage inhibition, we overexpressed WT and the uncleavable ALK LF655del CSM in SK-N-AS and CHP-212 human NB cells, that normally express very low levels of ALK (FIG. 11, panel B). After ascertaining cell surface expression, we determined that ALK WT underwent ECD cleavage in these cells, while ALK LF655del CSM failed to do so (FIG. 11, panels C and D). Again, no differences in downstream signaling were observed between cells expressing the cleaved and uncleavable receptors, indicating that ALK cleavage does not affect downstream signaling in these tumor cells that already possess established canonical signaling pathways.

### Inhibition of ALK ECD Shedding Limits the Migration but not Growth of NB Cells

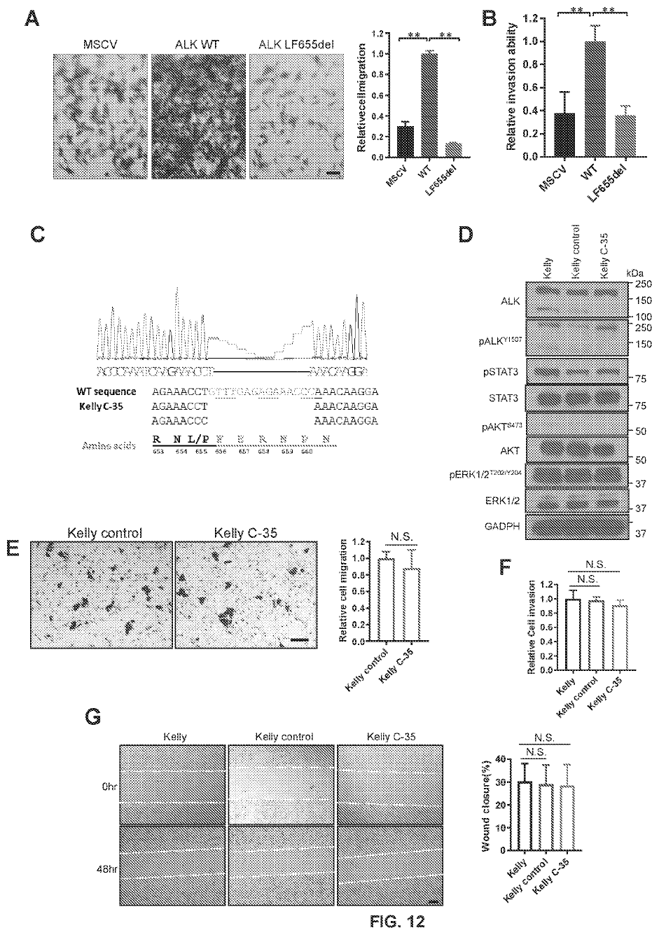

To determine whether ALK ECD shedding had a functional role in NB cells, we first assessed the effect of cleavage inhibition on cell growth. Uncleavable ALK-expressing NGPALK(LF655del) cells exhibited no changes in cell growth or cell cycle progression as compared with NGPCRISPR ctrl cells (FIG. 11, panels E-G). Given prior reports of the role of WT ALK in the migration and invasion of NB cells (Hasan et al., 2013), we next determined whether ECD cleavage featured in these processes. NGPALK(LF655del) cells exhibited a 3-fold reduction in migration compared with NGPCRISPR ctrl cells (FIG. 3, panel A), which could be rescued by overexpression of cleavable WT ALK. Inhibition of ECD cleavage also decreased the invasion and wound healing capacities of NGPALK(LF655del) cells compared with NGPCRISPR ctrl cells (FIG. 3, panels B and C). Similar to the effects on migration, these deficits were also overcome by the ectopic expression of WT ALK in these cells. We also analyzed these effects in additional non-transformed (NIH3T3) and human NB (SK-N-AS, CHP-212) cells engineered to express WT or the uncleavable ALK LF655del CSM (FIG. 3, panels D and E; FIG. 12, panels A and B). WT ALK expression resulted in the potentiation of cell migration and invasion while ALK LF655del had no effect on these processes. These results indicate that ECD cleavage of ALK contributes to the migratory and invasive properties of NB cells that express WT ALK. Finally, to determine whether ALK ECD cleavage has a functional role in NB cells expressing gain-of-function ALK mutations, we next introduced an ALK cleavage site mutation into Kelly NB cells that endogenously express the F1174L kinase mutation (George et al., 2008) through CRISPR editing (FIG. 12, panels C and D). Neither migration, invasion nor wound healing was affected in these cells (FIG. 12, panels E and G), without wishing to be bound by theory, because the constitutive kinase activity of ALK F1174L plays a dominant role in mediating cell motility, which overcomes the effects caused by ALK ECD cleavage inhibition.

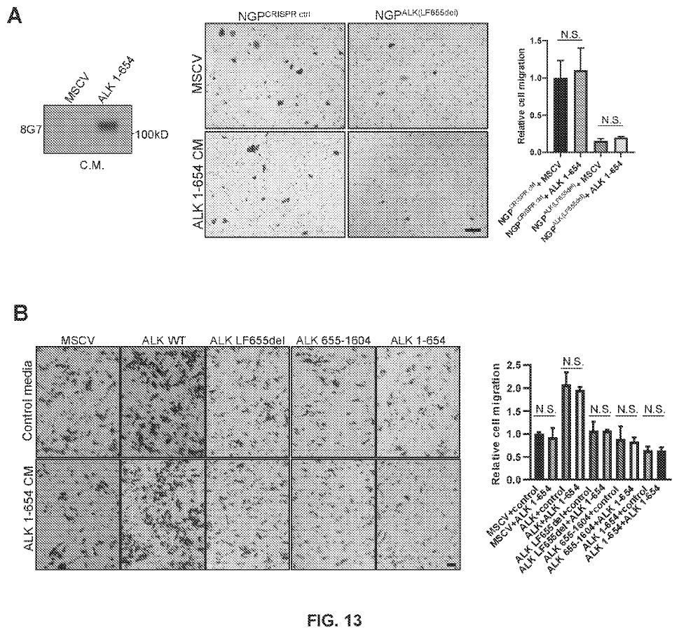

We next considered whether the effects on cell migration were mediated by the truncated membrane-bound ALK receptor that remains after ECD cleavage. We therefore overexpressed the ALK 655-1604 fragment together with an N-terminal membrane-localizing signal peptide (Hallberg and Palmer, 2016) in SK-N-AS and CHP-212 NB cells. After verifying that ALK 655-1604 was presented to the cell membrane (FIG. 11, panel C), we determined that overexpression of this fragment did not recapitulate the enhanced cell migration observed with cleaved WT ALK (FIG. 3, panel D and E). Similarly, incubation of NGPALK(LF655del) or CHP-212 NB cells ectopically expressing LF655del or ALK 655-1604 in conditioned media containing the shed ALK (ALK 1-654) ECD fragment had no effect on migration potential (FIG. 13, panels A and B). These results indicate that cleavage of the full-length ALK receptor but not the individual truncated or shed fragments is required to induce cell migration.

### Inhibition of ALK ECD Cleavage Decreases NB Cell Migration in Animal Models

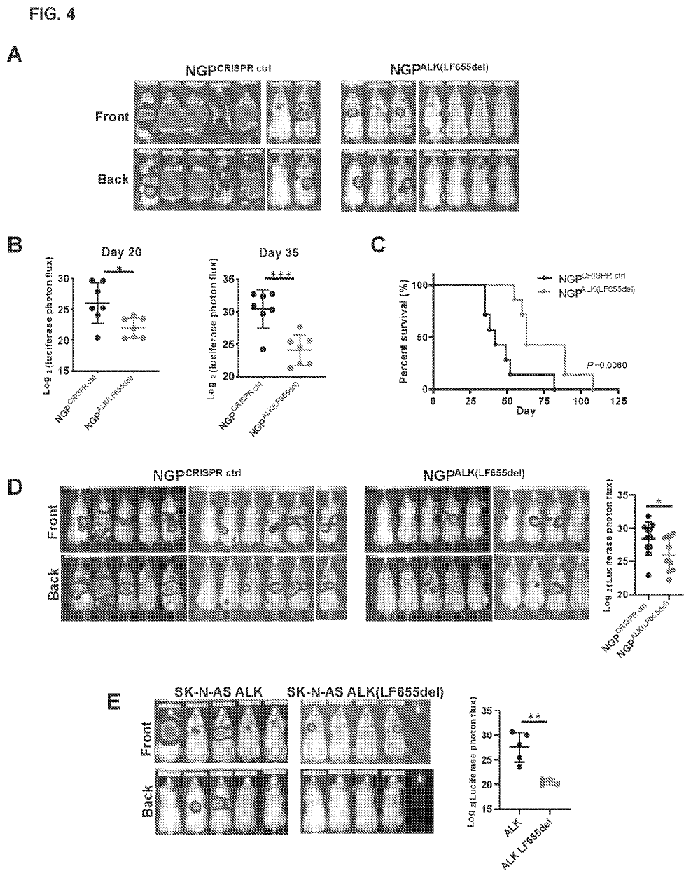

To validate the role of ALK cleavage in NB cell migration in vivo, we next compared the migratory potential of NGPALK(LF655del) NB cells in which ALK ECD cleavage was endogenously inhibited versus those in which WT ALK was cleaved (NGPCRISPR ctrl). We used an intracardiac murine model of NB metastasis in which cells metastasize to the bone marrow and bone with high penetrance, similar to the pattern of spread for NB cells in patients (Seong et al., 2017). Luciferase-labeled cells were injected into 9-week-old NOD/SCID mice and monitored for evidence of spread using bioluminescence imaging. At day 35 post injection, the latest time point at which each group had an equal number of animals, mice in which NGPALK(LF655del) cells were introduced showed a significant decrease in the total metastatic burden compared to mice injected with NGPCRISPR ctrl cells (FIG. 4, panels A and B). Between days 35 and 82 after cell injection, all NGPCRISPR ctrl tumor mice had to be euthanized due to widespread disease, in comparison to NGPALK(LF655del) animals that required euthanasia between 52 and 108 days. Thus, there was a delay in metastatic colonization (35 vs. 52 days) of tumor cells expressing uncleavable ALK. Importantly, the animals in which ALK ECD cleavage was inhibited showed a significant increase in survival (FIG. 4, panel C). As previously described for the intracardiac model of NB cell metastasis (Seong et al., 2017), NGPCRISPR ctrl cells (that express endogenous WT ALK) were seen in the bone marrow/bone and adrenal glands, with no difference in metastatic sites between the two groups. To ensure that our results were not due to the specific model or method of in vivo tumor generation, we also tested the metastasis-forming ability of NGPALK(LF655del) and NGPCRISPR ctrl xenograft models generated through tail vein injection (FIG. 4, panel D). This in vivo study also recapitulated the significantly reduced metastatic potential of NGPALK(LF655del) cells. At day 42 post injection, mice that had received NGPALK(LF655del) cells showed significantly decreased total metastatic burden compared with controls. With tail vein injections, most of the tumor cells were detected in the liver. Finally, we also tested the effect of ALK cleavage on cell metastasis in an additional NB mouse model using SK-N-AS NB cells ectopically expressing either WT or the LF655del CSM (FIG. 4, panel E). Tumor xenograft models derived from intracardiac injection of luciferase-labeled SK-N-AS cells expressing the CSM showed significantly decreased metastasis compared with WT ALK-expressing control animals. These in vivo results indicate a key role for ALK ECD cleavage in NB cell migration.

### ALK ECD Cleavage Leads to an EMT Gene Expression Signature

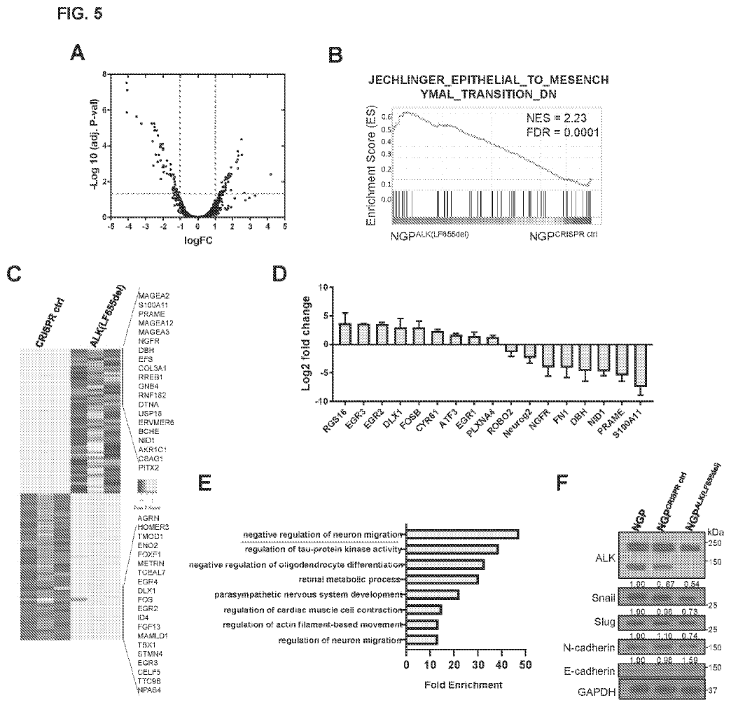

Given the absence of signaling alterations in ALK cleavage-inhibited cells, we next sought alternative mechanisms to explain the observed defects in adhesion and migration by first comparing the gene expression profiles of NGPALK(LF655del) with those of NGPCRISPR ctrl cells. While most transcripts were unchanged between the two cell types (FIG. 5, panel A), gene set enrichment analysis (GSEA) showed significant enrichment for genes involved in EMT in NGPALK(LF655del) cells (FIG. 5, panel B), with these genes featuring prominently among the most downregulated transcripts (FIG. 5, panels C and D). Further, gene ontology (GO) analysis identified genes involved in neuronal migration as the most enriched category (FIG. 5, panel E). The downregulated EMT signature was also reflected in the protein expression levels of candidate genes with key roles in EMT (Lamouille et al., 2014) (FIG. 5, panel F). Hence, inhibition of ALK ECD cleavage contributed to the loss of an EMT signature, thereby leading to effects on cell migration.

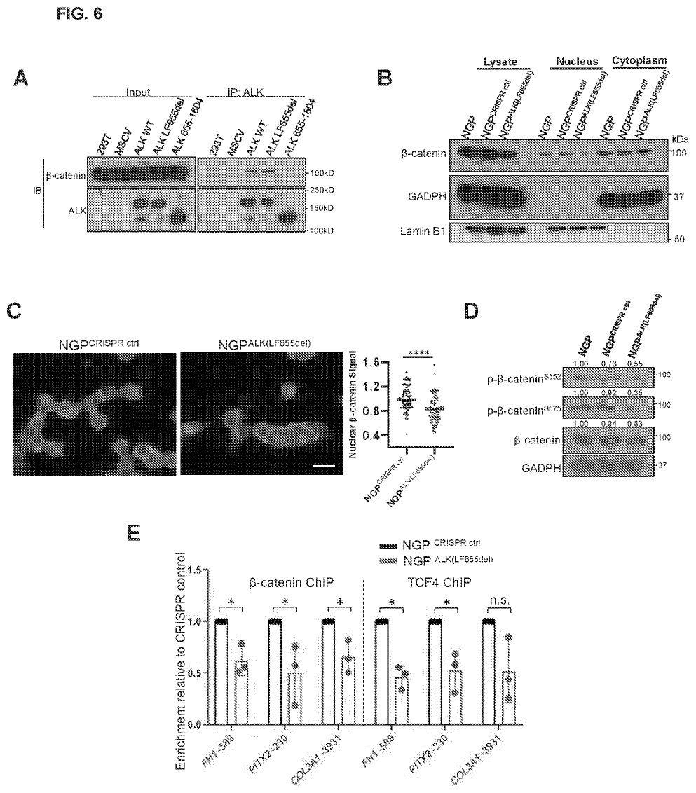

The Wnt/β-catenin signaling pathway is one of the key modulators of EMT (Thiery et al., 2009; Valenta et al., 2012). In addition to its role in signaling, β-catenin is normally sequestered and stabilized at the cell membrane as a structural component of cadherin-based adherence junctions (Meng and Takeichi, 2009). Release of β-catenin from its binding partners can lead to its nuclear translocation, where it drives the expression of target genes including those involved in the regulation of EMT. Interestingly, β-catenin was recently found to bind to the intracellular domain of ALK, causing steric hindrance to ALK inhibitor binding and subsequent resistance to therapy (Alshareef et al., 2017; Alshareef et al., 2016). This evidence, together with our observation that the top differentially downregulated genes in NGPALK(LF655del) cells included targets of β-catenin involved in EMT (FIG. 5, panels C and D), led us to question its role in mediating the migratory effects of ALK ECD cleavage. Co-immunoprecipitation assays using a C-terminal anti-ALK antibody showed that β-catenin binds to the intracellular domain of WT ALK (FIG. 6A), and that the binding is retained when ECD cleavage is inhibited. On the other hand, binding of β-catenin to the truncated membrane-bound ALK 655-1604 receptor was significantly reduced, suggesting that ECD cleavage could release β-catenin from cytoplasmic ALK-bound β-catenin, thereby enabling β-catenin to be transported to the nucleus. In support of this, a decrease in the levels of nuclear vs. cytoplasmic β-catenin in NGPALK(LF655del) cells was seen compared to those in untransfected NGP or NGPCRISPR ctrl cells (FIG. 6, panels B and C). Furthermore, phosphorylation of β-catenin at serines 552 and 675, which helps to maintain the stability of the protein and enables transcriptional co-activator binding (van Veelen et al., 2011), was decreased in NGPALK(LF655del) cells, prompting us to analyze β-catenin function in the nucleus (FIG. 6, panel D). ChIP-qPCR analysis revealed significantly diminished occupancies of β-catenin and its nuclear partner TCF-4, at the promoters of EMT genes, fibronectin 1 (FN1) (Zirkel et al., 2013), PITX2 (Kioussi et al., 2002) and COL3A1 (Xiang et al., 2017) in NGPALK(LF655del) cells compared to NGPCRISPR ctrl cells (FIG. 6, panel E). Taken together, these results indicate that ALK ECD shedding can contribute to NB cell migration through the release of ALK-bound β-catenin, leading to its nuclear localization and subsequent activation of EMT gene expression.

### The MMP-9 Protease Induces ALK ECD Cleavage

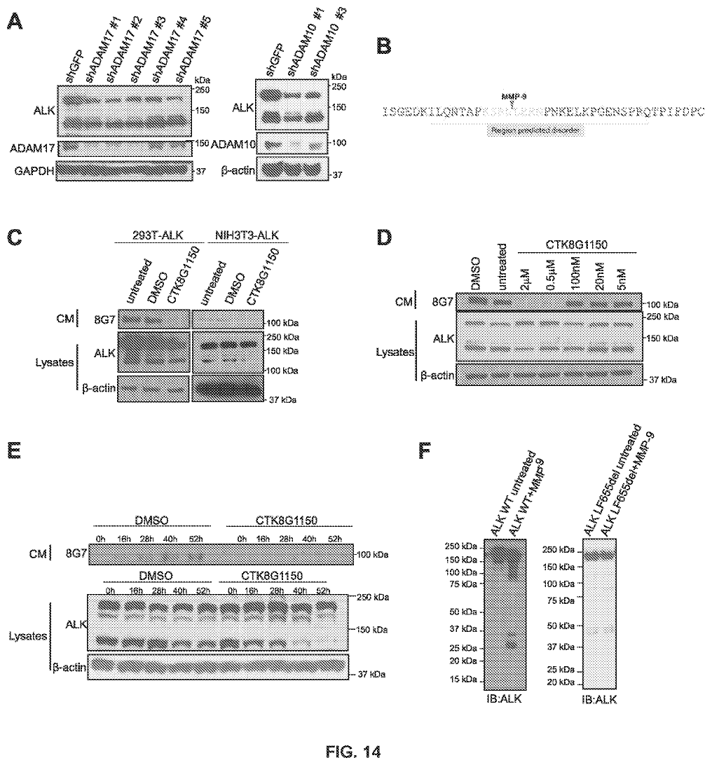

Most receptor tyrosine kinases (RTKs) undergo ECD cleavage by proteases known as sheddases, that include matrix metalloproteinases (MMPs) and A disintegrin and metalloproteinases (ADAMs) (Kreitman et al., 2018). These zinc-dependent, membrane-associated or secreted proteases cleave cell surface transmembrane proteins that play a primary role in the degradation of extracellular matrix proteins, thus affecting multiple biological functions, such as cell signaling, growth factor activation and cytokine release. To identify the protease(s) that mediate cleavage, we first validated ADAM family members that have been implicated in RTK cleavage, including ADAMs 10 and 17, that induce cleavage of AXL, MET and ErbB4 (O'Bryan et al., 1995; Rio et al., 2000; Schelter et al., 2010). shRNA-mediated knockdown of these proteases did not affect ALK cleavage (FIG. 14, panel A), leading us to test the effects of MMP inhibition in cells expressing WT ALK. The broad-spectrum protease inhibitors GM6001 (affecting MMP-1, MMP-2, MMP-3, MMP-8 and MMP-9) and CAS 204140-01-2 (MMP-9 and MMP-13) caused a decrease in the levels of the shed ALK ECD fragment in media from treated cells (FIG. 7, panel A), while MMP408 (MMP-3, MMP-12, MMP-13, but not MMP-9) (Li et al., 2009) did not affect cleavage (FIG. 7, panel B). The selective MMP-9 inhibitor CTK8G1150 (MMP-9, MMP-1 and MMP-13) (Levin et al., 2001), however, produced a marked decrease in the shed fragment, thereby narrowing the protease to MMP-9 (FIG. 7, panel B). These results were further substantiated by in silico protease substrate analysis that indicated the ALK cleavage site to contain the consensus substrate sequence for MMP-9 (FIG. 14, panel B) (Kumar et al., 2015; Song et al., 2012). CTK8G1150 treatment also led to significant inhibition of ECD cleavage in 293T and NIH3T3 cells engineered to overexpress WT ALK, with a commensurate decrease in the shed fragment. ALK ECD cleavage was disrupted in a dose-dependent manner and was maintained over a relatively prolonged period (>52 hr.) (FIG. 14, panel D and E). Importantly, the same compound also inhibited ECD cleavage in endogenous WT ALK-expressing NGP NB cells, a result that was replicated in four additional NB cell lines (FIG. 7, panel C). We next determined whether ALK could be cleaved by MMP-9 in vitro (FIG. 14, panel F). Treatment of purified WT ALK, but not the ALK LF655del protein, with recombinant human MMP-9 resulted in ALK cleavage. Finally, to confirm the role of MMP-9 in ALK cleavage, we genetically depleted this protease through shRNA knockdown in two NB cell lines [NGP and BE(2)-C], observing a significant decrease in the 140 kDa product resulting from inhibition of cleavage (FIG. 7, panel D). Depletion of MMP-2, by contrast, led to only a slight decrease in cleavage. Importantly, treatment with the MMP-9 inhibitor significantly suppressed the migration of NB cells that endogenously expressed WT ALK (FIG. 7, panel E). Together, these results identify MMP-9 as the mediator of ALK ECD cleavage, whose impact on NB cell migration can be abolished by treatment with a specific MMP-9 inhibitor.

### Discussion

Ectodomain cleavage is a feature of many transmembrane receptor tyrosine kinases (RTKs), where shedding leads to activation or repression of the receptor (Kreitman et al., 2018). In breast cancer, ECD cleavage of the HER2 receptor generates membrane-bound p95-HER2 that imparts growth and survival signals to the cell (Liu et al., 2006). By inhibiting such cleavage, the anti-HER2 antibody trastuzumab disrupts HER2 signaling and blocks cell cycle progression, improving patient outcome (Molina et al., 2001). Similarly, the AXL receptor, whose upregulation arises as a mechanism of resistance to various kinase inhibitors, including ALK (Debruyne et al., 2016; Zhang et al., 2012) also undergoes ECD cleavage, however this process in certain cells represses AXL and slows tumorigenesis (Miller et al., 2016). Here we show that ALK undergoes ECD cleavage at Asn654-Leu655 mediated by the MMP-9 matrix metalloprotease and thereby promotes the migration and invasion of human NB cells.

We demonstrate that inhibition of ECD cleavage in NB cells that endogenously express ALK leads to the suppression of cell migration and invasion, which can be rescued by overexpression of WT cleavable ALK. Importantly, exogenous expression of the WT cleavable receptor in both NB and non-NB cells that express minimal or no ALK results in significantly increased cell migration, an effect that was not noted with the cleavage-site mutants. These observations indicate a prominent role for ECD shedding of the ALK receptor in cell migration, which is also reflected in the enrichment of transcripts involved in neuronal migration in cleavage-inhibited NB cells. Downregulation of EMT signatures and especially the differential expression of critical proteins that regulate cell migration are consistent with the failure of these cells to metastasize in vivo. ALK ECD cleavage also occurs in early postnatal mouse brain cells. During development, activation of an EMT program is essential for the migration of cells that form the neural crest, enabling their dispersion to multiple sites throughout the body, including sympathetic ganglia. Together with the highly conserved ALK cleavage site across different mammalian species, these observations indicate that ECD shedding is critical for neural crest cells to undergo EMT and migrate during development, and when aberrantly expressed in NB cells, potentiating tumor cell invasion and metastasis. The recent finding of structural genomic alterations that result in deletion of the N-terminal domain in 2.4% of primary patient samples reinforces the functional relevance of this phenomenon.

Whether the increased migration potential following ALK ECD cleavage is regulated by the truncated membrane-bound receptor that remains after ECD shedding or the shed ECD is not entirely clear, as exposure to either fragment failed to increase cell migration. Alternatively, the retained binding of cleavage-inhibited ALK to β-catenin and lower nuclear β-catenin levels, together with the decreased occupancy of β-catenin at key EMT gene promoters offer a link between ALK ECD cleavage and EMT. Full-length ALK binds to β-catenin (Alshareef et al., 2017; Alshareef et al., 2016) (and this study), and therefore, shedding of the ECD fragment through cleavage can disrupt the interaction between the membrane-bound, truncated ALK intracellular protein and β-catenin, leading to the release of β-catenin and enabling its nuclear translocation and transcription of genes involved in cell migration. β-catenin also undergoes nuclear translocation during development in neural crest cells where it activates SNAIL and SLUG EMT gene transcription, which in turn repress E-cadherin expression (Barrallo-Gimeno and Nieto, 2005; Shoval et al., 2007). Whether these events contribute to neural crest cell migration requires further study. ALK ECD shedding can also be a prerequisite for subsequent intracellular cleavage as described for other heavily glycosylated proteins, such as trophoblast cell-surface antigen 2 (Trop2) and epithelial cell adhesion molecule (EpCAM) (Maetzel et al., 2009; Stoyanova et al., 2012). Indeed, caspase-dependent cleavage at the juxtamembrane intracytoplasmic region of the ALK receptor has been shown to cause anti- or pro-apoptotic effects depending on the presence or absence of ligand, respectively (Mourali et al., 2006). Alternatively, cleavage can attenuate ALK activity and/or cell surface expression through competitive ligand binding of the shed ECD.

ALK ectodomain shedding did not affect its phosphorylation at Y1507 and Y1604, nor did it appear to affect its kinase activity. For example, without wishing to be bound by theory, the ectodomain cleavage does not remove the ligand binding region, so that the receptor can still be activated through ligand binding. Also, for example, the kinase domain activation of ALK and attendant growth promoting downstream signaling can be separate from its role in the regulation of EMT genes, as reflected by the unchanged cell growth and colony formation seen with loss of ECD cleavage as well as the lack of effect on downstream signaling. However, downstream signaling was decreased in NIH3T3 cells expressing ALK cleavage-site mutations. A reason for this discrepancy includes the not contribution of other dominant oncogenic stimuli in the NB cells such as MYCN amplification. Also, downstream signaling in ALK ECD cleavage-inhibited NB cells was indeed initially downregulated but was restored through the establishment of compensatory mechanisms.

Without wishing to be bound by theory, MMP-9 as the protease that cleaves the ECD of ALK. However, ALK ECD can be cleaved by other proteases such as MMP-2 (Kumar et al., 2015; Song et al., 2012). In addition, this sequence can also represent cleavage itself or plays a critical role in maintaining the site in a confirmation that is permissive for MMP-9-mediated cleavage. MMP-9, together with MMP-2, are involved in extracellular matrix remodeling during many biological processes, including invasion, neurite growth, and embryonic development (Vandooren et al., 2013). In NB, stromal cells (such as vascular cells or macrophages) are major sources of MMP-9 (Sugiura, 1998; Chantrain, 2004). Activation of extracellular MMP-9 activity triggers cell motility, not only because of cleavage of its target molecules within the plasma membrane or extracellular matrix (such as cell adhesion molecules), but also because MMP-9 can efficiently degrade the extracellular matrix, an important prerequisite for metastasis (Tanjore and Kalluri, 2006). Moreover, MMP-9 has been shown to cooperate with SNAIL to potentiate cell migration (Lin et al., 2011), and SNAIL in turn, positively regulates MMP-9 expression (Jorda et al., 2005). Incubation of NB cells in Schwann cell media abolishes ALK cleavage (Degoutin et al., 2009), for example, due to the production of natural MMP-9 inhibitors by Schwann cells, such as tissue inhibitor of metalloproteinase, TIMP-1 and TIMP-2 (Brew and Nagase, 2010; Huang et al., 2000; Kim et al., 2012) and Schwannian stroma-rich NBs are characteristically non-metastatic. The selective MMP-9 antibody (andecaliximab; Gilead, GS-5745) achieved target engagement without dose-limiting toxicity and showed activity when combined with standard chemotherapy in patients with gastric and gastroesophageal junction adenocarcinoma (Shah et al., 2018), and can be a therapeutic strategy that could be tested in patients with NB.

In conclusion, the discovery of activating mutations in the ALK receptor over a decade ago has led to numerous studies to understand the impact of aberrant ALK signaling on NB pathobiology and to devise clinically effective countermeasures. These efforts have yielded a series of ALK inhibitors with the ability to silence oncogenic ALK expression and block the growth of NB cells in experimental models (Hallberg and Palmer, 2016), but to date this progress has not translated to clear therapeutic gains in patients (Foster et al., 2021). Although newer generations of inhibitors can be more effective, success with direct targeting of ALK kinase activity would benefit only the approximately 10% of high-risk NB patients who have tumors harboring activating mutations, leading to efforts to harness ALK as a tumor-associated antigen. Here we show that proteolytic cleavage of the ECD of WT ALK which is expressed in a large majority of NB cases promotes metastasis and that by targeting this process, these adverse cancer-associated features of ALK cleavage can be circumvented, indicating that MMP-9 inhibition either singly or in combination with that of ALK in patients with NB. The identification of the cleavage site in both ALK WT and mutated cells has additional implications for immunotherapies based on expression of the full-length receptor on the cell surface, as antibodies and/or other strategies specific to epitopes in the shed ECD would not be able to bind to the truncated membrane-bound receptor. Blockade of ECD shedding by co-treatment with an MMP9 inhibitor could be utilized as a strategy to increase the available expression of full-length ALK on the cell surface, thereby also enabling NK cell effector activity. The shed ALK ECD also could be used as a biomarker to non-invasively and longitudinally monitor disease activity in patients with NB. Finally, it will be important to determine whether ALK ECD cleavage also occurs in the other cancers in which the full-length receptor is expressed and whether its effects recapitulate our findings in NB.

### In Vivo Metastasis Study.

Animal experiments were performed on an approved Toronto SickKids Institutional protocol. NGP (NGP^{CRISPR ctrl} or NGP^{ALK(LF655del)}) or SK-N-AS (SK-N-AS WT ALK cells or SK-N-AS ALK LF655del) neuroblastoma cells were infected with the retroviral TGL (thymidine kinase-GFP-luciferase) reporter to generate stable cell lines (Ponomarev et al., 2004). GFP-positive cells were selected through FACS. Luciferase activity of sorted cells was confirmed using the Luciferase Assay System (Promega). 1×10⁵ cells were introduced into 9-week-old NOD/SCID mice obtained from the University Health Network Immune-deficient Mouse Colony (Toronto), via intracardiac injections as previously described (Kang et al., 2003) (Seong et al., 2017). Animals were monitored for health, weight, appearance, and metastatic burden and sacrificed following the SickKids Institutional Animal Utilization Protocol. For imaging, mice were injected with D-luciferin (PerkinElmer) and imaged 10 and 12 minutes post-injection with the Xenogen IVIS imaging system. Bioluminescence was quantified by drawing a region of interest (ROI) around each animal and measured as photon flux per second. For the tail vein injection model, 6-8-week-old NOD/SCID mice were injected in the lateral tail vein with 5×10⁵ cells in PBS. The animals were monitored and bioluminescence imaged. Endpoints were reached at whole body signal saturation and/or signs of morbidity.

### Preparation of Tissue Samples.

All mice experiments were performed with approval from the Institutional Animal Care and Use Committee (IACUC) of the DFCI. Neuroblastoma Patient-derived xenografts (PDX) were received from St. Jude Children's Research Hospital and expanded in nude mice (Crl:NU-Foxn1nu) obtained from Charles River Laboratory. Mice brain tissues were collected from C57BL/6J mice and stored at −80° C. after snap freezing in liquid nitrogen. Frozen PDXs and brain tissues were homogenized with electric homogenizer in cold NP-40 buffer (Invitrogen) containing complete protease inhibitor (Roche), phosphatase inhibitor (Roche), and PMSF. Lysates were incubated on ice for 20 min and centrifuged at 16,000 g for 15 min at 4° C. The supernatants were applied for western blotting.

### Cell Culture.

Human neuroblastoma (NB) cell lines (NGP, IMR5, Kelly, NBL-S, SH-SY5Y, SK-N-AS, CHP-212) were obtained from the Children's Oncology Group cell line bank. The BE (2)-C, NIH3T3 and HEK293T cells were purchased from the American Type Culture Collection (ATCC). Cell lines were authenticated by genotyping at the DFCI Core Facility. NB cell lines were grown in RPMI-1640 medium supplemented with 10% fetal bovine serum (FBS) and 1% penicillin/streptomycin (Invitrogen). HEK293T cells were grown in DMEM medium supplemented with 10% FBS and 1% penicillin/streptomycin. NIH3T3 cells were grown in DMEM with 10% fetal calf serum (FCS; Sigma-Aldrich) and 1% penicillin/streptomycin. Cells were cultured at 37° C. in 5% CO₂. Cell lines from male include NGP, BE (2)-C, IMR5, NBL-S, CHP-212, NIH3T3. Cell lines from female include Kelly, SH-SY5Y, SK-N-AS, HEK293T.

### Western Blotting.

Cell lysates were prepared in NP-40 buffer (Invitrogen) containing complete protease inhibitor (Roche), phosphatase inhibitor (Roche) and phenylmethylsulfonyl fluoride (PMSF). Lysates were incubated on ice for 20 min and centrifuged at 16,000 g for 15 min at 4° C. Protein concentrations of the supernatants were determined with the DC protein assay kit (Bio-Rad). Protein samples were denatured using NuPAGE LDS sample buffer (Invitrogen) and NuPAGE sample reducing agent (Invitrogen). Proteins were then separated on precast 4-12% Bis-Tris gels (Invitrogen) and transferred to nitrocellulose membranes (Bio-Rad). After blocking in blocking buffer (5% dry milk in TBS with 0.2% Tween-20), membranes were incubated with the primary antibody overnight at 4ºC, washed three times with TBST buffer, chemiluminescent detection performed with the appropriate secondary antibodies and developed using Genemate Blue ultra-autoradiography film (VWR).

### Mass Spectrometry (MS) Sample Preparation.

NGP cells were grown in serum-free RPMI-1640 medium for 72 hours. Conditioned medium was collected and concentrated (more than 40×) using an ultra-15 centrifugal filter (Amicon) at 4º C. The concentrated medium was then subjected to co-immunoprecipitation (co-IP) using the N-terminal 8G7 ALK antibody. Proteins were separated on SDS-PAGE gels (Thermo Fisher Scientific; 4%-12% Bio-Tris gel) before staining with Coomassie Blue R-250 (Bio-Rad). The band corresponding to the ALK ectodomain was excised, sequentially washed with 50% methanol/water and 47.5/47.5/5% methanol/water/acetic acid, dehydrated with acetonitrile, reduced with DTT and alkylated with iodoacetamide. The sample was then sequentially washed with 50 mM ammonium bicarbonate/50% acetonitrile and acetonitrile, and enzymatically digested with trypsin or chymotrypsin. The digested sample was injected onto a C18 trap column and eluted onto an analytical column (Jupiter C18 column, Phenomenex). The digested extracts were analyzed by reversed-phase high-performance liquid chromatography (HPLC) (C18 column) and Tandem MS using an Orbitrap Elite mass spectrometer (Thermo Fisher Scientific). Peptides were identified from the MS data using SEQUEST algorithms. A species-specific database generated from the NCBI non-redundant database (nr.fasta) was used to identify the peptides. The resulting data were then loaded onto Scaffold (Proteome Software, Portland, OR) for analysis. A peptide threshold of 95% was used for identification of peptides.

### Site-Directed Mutagenesis.

Site-directed mutagenesis was performed using a PCR-based strategy. Point mutations within the plasmid construct were introduced by site-directed mutagenesis using the QuikChange II Site-Directed Mutagenesis Kit (Agilent Technologies) following the manufacturer's instructions. The resulting mutant plasmids were verified by DNA sequencing. ALK 655-1604 was generated using the Q5 site Mutagenesis Kit (NEB) following the manufacturer's instructions.

### Stable Cell Line Preparation.

The retrovirus was packaged by cotransfection of pLKO.1 shRNA construct and the helper plasmids pMD.MLV and pMD2.G-VSV-G into HEK293T cells using TransIT-LT1 Transfection Reagent (Mirus). The virus-containing supernatant was harvested and filtered through a 0.45 μm polyvinylidene fluoride (PVDF) filter (VWR) and applied to cells in the presence of polybrene (10 μg/ml). After 48 hours, cells were selected with puromycin (NIH3T3 cells 2 μg/ml; NGP cells 2.5 μg/ml; SK-N-AS cells 2 μg/ml; CHP-212 cells 1 μg/ml; 293T cells 2 μg/ml) or fluorescence-activated cell sorting (FACS).

### Cell Surface Protein Analysis.

Cell surface protein isolation was conducted using a cell-surface protein isolation kit (Pierce) according to the manufacturer's instructions with minor modifications. Biotinylated proteins were isolated with neutravidin agarose resin (Thermo Scientific) which was packed in an isolation column. After the biotinylated proteins were eluted with the SDS-containing sample buffer, proteins were analyzed by SDS-PAGE and western blotting.

### Glycosylation Assay.

The Peptide N-Glycosidase F (PNGase F) deglycosylation assay (New England Biolabs) was performed according to the manufacturer's instructions. Cell lysates from NGPCRISPR ctrl or NGP^{ALK(LF655del)} cells prepared with NP-40 buffer were denatured at 100° C. for 10 minutes in the presence of glycoprotein denaturing buffer and incubated with PNGase F in GlycoBuffer (1×) at 37° ° C. for 1 hr, following which ALK deglycosylation was analyzed through immunoblotting with anti-ALK antibodies.

### FLAG Fusion Protein Purification.

The MSCV-FLAG-ALK or MSCV-FLAG-ALK LF655del expression constructs were transiently transfected into 293T cells using TransIT-Express Transfection Reagent (Mirus) to express ALK proteins. Recombinant wild-type ALK or mutant ALK proteins were purified using an anti-FLAG purification kit (Sigma-Aldrich) according to the manufacturer's instructions. The affinity purification was performed using an anti-Flag M2 affinity gel, a highly specific monoclonal antibody covalently attached to agarose resin.

### Co-Immunoprecipitation (Co-IP).

HEK293T cells were transiently transfected with WT and mutant ALK constructs to express HA-tagged and FLAG-tagged proteins. Cells were washed with PBS and harvested in IP lysis buffer (50 mM Tris-HCL buffer (pH 7.4), 100 mM NaCl, 1% Triton-100, 1 mM PMSF), containing the cocktail protease inhibitor (Roche) and phosphatase inhibitor (Roche). Homogenates were centrifuged at 20,000 g for 10 min at 4° C. Supernatants were collected and co-IP performed using Dynabeads protein G (Life Technologies) according to the manufacturer's instructions. Briefly, Dynabeads were incubated with either anti-FLAG or anti-HA antibody, washed to remove unbound primary antibody, and incubated with cell lysates for immunoprecipitation of the target antigen. The elution step was performed by heating the beads for 10 min at 95° C. in premixed NuPAGE LDS sample buffer and NuPAGE sample reducing reagent (Invitrogen). Samples were then loaded onto NuPAGE 4-12% Bis-Tris protein gels (Invitrogen), proteins transferred to nitrocellulose membranes (Bio-Rad) and immunoblotting performed as described above.

### In Vitro Kinase Assay.

The Universal Tyrosine Kinase Assay kit (Takara) was used to measure kinase activity according to the manufacturer's instructions. Briefly, NIH3T3 cells stably expressing WT ALK or ALK CSMs were harvested in extraction buffer, lysates incubated on ice for 20 min, centrifuged at 10,000 g for 10 min at 4° C. and the supernatants pre-cleared with 50 μl protein G agarose beads for 30 min at 4° C. under rotation. 10 μl ALK antibody was then added to the supernatant and incubated at 4° C. under rotation for 4 hr. 30 μl protein G agarose beads were then added to the supernatant and incubated at 4° C. under rotation for 2 hr. The immunoprecipitated material was then washed with PBS and used in the kinase assay.

### Preparation of ALK Ligand-Conditioned Medium.

HEK293 cells were grown in 10 cm dishes to 90% confluency and transfected with 10 μg each of the following plasmids: pcDNA3 vector control, pTT5-FAM150A-HA or pcDNA3-FAM150B-HA with lipofectamine 3000 following the manufacturer's instructions (Invitrogen). After 12 hr., the medium was replaced with serum-free medium. After 24 hr. incubation at 37° C. in 5% CO₂, the conditioned medium was harvested for the experiment. 1 ml of harvested medium (control, ALKAL1- or ALKAL2-conditioned) was added to 6-well plates of NIH3T3 cells stably expressing WT or mutant ALK, incubated for 1 hr. at 37° C. in 5% CO₂, harvested and used in WB analysis of ALK and mutant ALK activation.

### Expression of Human ALK Mutants in the Drosophila Eye.

The Gal4-UAS system was applied to ectopically express human ALK constructs within the Drosophila eye. Drosophila stocks w¹¹¹⁸ and GMR-Gal4 (stock number 5905 and 9146, respectively) from Bloomington Drosophila Stock Center (Indiana University) were used. Generation of the Drosophila transgenic UAS-ALK and UAS-ALKAL2(FAM150A) lines were described previously (Guan et al., 2015; Schonherr et al., 2012). UAS-ALK LF655del was synthesized (GenScript). Transgenic flies were obtained by injection (BestGene Inc.). Transgenic Drosophila lines carrying the UAS-ALK, UAS-ALKAL2, UAS-ALK LF655del or UAS-ALK^{F1174L} were crossed with the GMR-Gal4 transgenic driver line to drive ectopic expression of the ALK variants in the eye imaginal discs. Experiments were conducted at 25° C. Adult flies were collected and frozen at −25° C. prior to microscopic analysis with a Zeiss AxioZoom V16 stereomicroscope.

### CRISPR/Cas9-Mediated Gene Editing.

Genomic editing experiments were conducted using CRISPR-Cas9 editing as previously described (Ran et al., 2013). A single guide RNA (sgRNA) was designed using the GPP sgRNA Designer Tool (Broad Institute). An ALK-single stranded oligonucleotide (ssODN), purchased from Integrated DNA Technologies (IDT), was used to introduce precise genomic editing through homology-directed repair (HDR). The guide RNA was first cloned into the BbsI site of the pSpCas9(BB)-2A-Puro (PX459) V2.0 vector (Addgene) to generate plasmid PX459-ALK-gRNA. NGP cells were then transiently transfected with this plasmid and ALK-ssODNs using Lipofectamine 3000 (Thermo Fisher Scientific). Cells were incubated at 37ºC for 30 hr., selected for 48 hours in 2.5 μg/ml puromycin, expanded, and diluted to single cells into 96-well plates. Single-cell clones were identified and allowed to expand for 6 weeks, following which cell editing was validated by sequencing. gRNA and ssODN sequences available on request.

### Immunofluorescence Analysis

Cells (0.5×10⁶ per well) were seeded into six-well plates and grown on glass coverslips for 24 hr. before fixation in 4% paraformaldehyde in PBS for 15 min at room temperature (RT), washed with PBS, permeabilized in 0.1% Triton X-100, and blocked with PBS containing 0.05% Tween-20 and 5% BSA for 1 hr. to block nonspecific binding. Then, cells were incubated with the 8G7 antibody (1:100) overnight at 4° C. After washing with PBS, cells were incubated with AlexaFluor 568-conjugated secondary antibody for 1 hr. at 25° C. Images were collected on a Zeiss AXIO Imager Z1 fluorescence microscope. For β-catenin immunofluorescence measurement, cells were plated onto poly-lysine coated cover slips in a 12-well plate. After 24 hours, cells were washed once in PBS, fixed in 4% paraformaldehyde in PBS, washed, permeabilized in 0.1% Triton X-100, and blocked in PBS containing 10% horse serum. Cells were incubated in β-catenin primary antibody (1:500) overnight. The next day, cells were washed, incubated in goat anti-rabbit IgG secondary antibody (1:1000) for 1 hr. washed, and incubated in Streptavidin-TexasRed (1:1000) for 1 hr. Cover slips were mounted onto slides using mounting medium containing DAPI. Images were collected on a Zeiss AXIO Imager Z1 fluorescence microscope.

### Cell Growth Assay.

Cells (1×10³ per well) were plated in 96-well plates on day 0 and analyzed for growth at intervals using the CellTiter-Glo Luminescent Cell Viability Assay (Promega) according to the manufacturer's instructions. The results were collected using the FLUOstar Omega microplate reader (BMG Labtech) and analyzed using MARS software (BMG Labtech).

### Focus Formation Assay.

Cells (1×10³ per well) were seeded into 6-well plates, incubated at 37° C. in 5% CO₂ for 14 days, with media replaced every 4 days. After 14 days, the medium was removed, cells were fixed with 100% methanol and stained with 0.5% crystal violet for 20 min at room temperature, rinsed with ddH₂O and focus formation analyzed by eye after air-drying.

### Soft Agar Colony Formation.

For soft agar assays, experiments were carried out in 24 well plates coated with a base layer of RPMI containing 0.5% agar, and 10% FBS. Cells (5×10³ per well) were seeded in RPMI containing 0.35% agarose, and 10% FBS and incubated for 14 days during which 0.5 ml fresh medium was added to each well every 5 days. After incubation for 14 days at 37° C. in 5% CO₂, plates were subjected to 0.005% crystal violet staining for 2 hr. Colony formation was analyzed using an IX70 inverted optical microscope (Olympus).

### RNA Extraction and Gene Expression Analysis.

NGP^{CRISPR ctrl} or NGP^{ALK(LF655del)} cells (8×10⁶ cells per replicate) were collected and total RNA extracted using TRIzol Reagent followed by purification using the mirVana™ miRNA Isolation Kit (Thermo Scientific) according to the manufacturer's instructions. The quality of all RNA samples was checked using NanoDrop 2000 (Thermo Scientific). RNA samples were spiked-in with ERCC RNA Spike-In Mix (Ambion) for expression normalization. PrimeView™ Human Gene Expression Array GeneChip (Affymetrix) was used for the gene expression assays. Preparation of cDNA, hybridization, and scanning of microarrays were performed at the DFCI core facility according to the manufacturer's protocols (Affymetrix).

### Microarray Data Analysis.

Microarray data were analyzed using a custom CDF file (GPL16043) that contained the mapping information of the ERCC probes used in the spike-in RNAs. The arrays were normalized as previously described (Loven et al., 2012). In brief, all microarray chip data were imported in R (https://www.r-project.org/, v.3.5.1) using the affy package (v.1.44.0) (Gautier et al., 2004), converted into expression values using the expresso command, normalized to take into account the different numbers of cells and spike-ins used and renormalized using loess regression fitted to the spike-in probes. Sets of differentially expressed genes were obtained using the limma package (v.3.22.7) (Smyth et al., 2003) and an FDR value of 0.05. Gene set enrichment analysis (GSEA) was performed using GSEA v3.0 software (Broad Institute). Significantly enriched gene sets were evaluated as per standard criteria (FDR≤0.25 and nominal P-value≤0.05). Gene ontology analysis was performed using the PANTHER classification system.

### Quantitative RT-PCR.

Total RNA was extracted from cells (8×10⁶ cells per replicate) using TRIzol Reagent followed by purification using the mirVana™ miRNA Isolation Kit (Thermo Scientific). cDNA was synthesized from 1 μg of purified RNA using SuperScript III reverse transcriptase system (Invitrogen) following the manufacturer's protocol. qPCR was performed using the QuantiFast SYBR Green PCR kit (Qiagen) with a Biosystems ViiA 7 Real-Time PCR System (Life Technologies). The housekeeping gene GAPDH was used as an internal control to normalize the variability in expression levels. The ΔΔCt relative quantification method was performed to measure relative quantitation of mRNA.

### Cell Invasion Assay.

The QCM™ ECMatrix™ Cell Invasion Assay Kit (Millipore) was used with minor modifications. Cells were incubated in serum-free medium for 18 hr. at 37° C. in 5% CO₂. A cell suspension containing 5×10⁵ cells/mL in serum-free medium was added to the upper chamber. Serum-containing medium was added to the lower chamber. NGP and NIH3T3 cells were incubated for 72 and 24 hr. respectively and the protocol followed as per the manufacturer's instructions.

### Cell Migration Assay.

Cell migration was measured using transwell chambers (Falcon; cell culture inserts with 8 μm pores) and a 24-well plate as the lower chamber. A cell suspension containing 0.5×10⁶ cells/mL in serum-free medium was added to the upper chamber. Inserts were placed in the lower chamber containing medium with serum. NGP, NIH3T3, CHP-212 and SK-N-AS cells were incubated at 37° C. in 5% CO₂ for 48, 16, 24, and 8 hr. respectively. After incubation, cells remaining on the top surface of the insert were removed. Migrated cells were fixed with methanol, stained with crystal violet (Sigma-Aldrich), and photographed with a light microscope at 100× magnification.

### Wound Healing Assay.

Cells were seeded into 6-well plates to form a cell monolayer. When 70-80% confluence was reached, the monolayer was scratched using a pipette tip. The scratched area at time point 0 hr. was set as the initial wound width. Cells were incubated in 5% CO₂ at 37° C., and the area not covered was measured at 24 to 48 hr. and photographed.

### In Vivo Metastasis Study.

Animal experiments were performed on an approved Toronto SickKids Institutional protocol. NGP cells (NGP^{CRISPR ctrl} cells or NGP^{ALK(LF655del)} cells) were infected with the retroviral TGL (thymidine kinase-GFP-luciferase) reporter to generate stable cell lines (Ponomarev et al., 2004). GFP-positive cells were selected through FACS. Luciferase activity of sorted cells was confirmed using the Luciferase Assay System (Promega). 1×10⁵ cells were introduced into 9-week-old NOD/SCID female mice obtained from the University Health Network Immune-deficient Mouse Colony (Toronto), via intracardiac injections as previously described (Kang et al., 2003) (Seong et al., 2017). Animals were monitored for health, weight, appearance, and metastatic burden and sacrificed following the SickKids Institutional Animal Utilization Protocol. For imaging, mice were injected with D-luciferin (PerkinElmer) and imaged 10 and 12 minutes post-injection with the Xenogen IVIS imaging system. Bioluminescence was quantified by drawing a region of interest (ROI) around each animal and measured as photon flux per second.

### ShRNA Knockdown.

pLKO.1 plasmid containing shRNA sequences targeting MMP-9 (sh #1: TRCN0000373008; sh #6: TRCN0000051438), ADAM10 (sh #1: TRCN0000006674; sh #3: TRCN0000006674), and ADAM17 (sh #1: TRCN0000052171; sh #2: TRCN0000052170; sh #3: TRCN0000052172; sh #4: TRCN0000052168; sh #5: TRCN0000052169) were purchased from Sigma-Aldrich. pLKO.1 GFP shRNA was a gift from D. Sabatini, MIT (Addgene plasmid 30323). The lentivirus was packaged by co-transfection of the pLKO.1 shRNA, pCMV-dR8.91, and pMD2.G-VSV-G constructs into HEK293T cells using the TransIT-LT1 Transfection reagent (Mirus). The virus-containing supernatant was filtered with a 0.45 μm PVDF filter (VWR). Cells were then transduced with virus, followed by puromycin selection for two days.

### In Vitro Cleavage Assay.

Purified recombinant ALK or ALK LF655del proteins were incubated with recombinant human MMP-9 (Biolegend) in reaction buffer (50 mM Tris-HCl buffer, pH 7.4, 150 mM NaCl) containing 0.2 mM p-aminophenylmercuric acetate (Calbiochem) for 3 hr. at 37° C. The samples were then denatured using NuPAGE LDS sample buffer and NuPAGE sample reducing reagent (Invitrogen), loaded onto SDS-PAGE (4%-12% Bio-Tris) gels and WB performed as described herein.

### Chromatin Immunoprecipitation-Quantitative PCR (ChIP-qPCR)

Approximately 10-12×10⁷ NGP^{CRISPR ctrl} or NGP^{ALK(LF655del)} cells were crosslinked with 1% formaldehyde (Thermo Scientific) for 10 min at room temperature (RT) followed by quenching with 0.125 M glycine for 5 min. The cells were then washed twice in ice-cold 1× Phosphate Buffered Saline (PBS), and the cell pellet equivalent of 4×10⁷ cells were flash frozen and stored at −80° C. Crosslinked cells were lysed in lysis buffer 1 (50 mM HEPES-KOH PH7.5, 140 mM NaCl, 1 mM EDTA, 10% glycerol, 0.5% NP40, 0.25% Triton X-100). The resultant nuclear pellet was washed once in lysis buffer 2 (10 mM Tris-HCl pH 8, 200 mM NaCl, 1 mM EDTA, 0.5 mM EGTA) and then resuspended in sonication buffer (50 mM HEPES-KOH PH 7.5, 140 mM NaCl, 1 mM EDTA, 1 mM EGTA, 1% Triton X-100, 0.1% sodium deoxycholate, 0.2% SDS). Chromatin was sheared using a Misonix 3000 sonicator (Misonix) and at the following settings: 9 cycles, each for 30 s on, followed by 1 min off, at a power of approximately 20 W. The lysates were then diluted with an equal amount of sonication buffer without SDS to reach a final concentration of 0.1% SDS, centrifuged at 4000 rpm for 15 min at 4° C., and supernatants containing soluble chromatin were collected. For each ChIP, the chromatin equivalent of 1×10⁷ cells was used. 10 μl of Protein G Dynabeads per sample (Invitrogen) were blocked with 0.5% BSA (w/v) in 1×PBS and were incubated with the following antibodies overnight at 4° ° C.: 0.36 μg of anti-β-catenin (Cell Signaling Technology 8480S) or 1 μg of TCF4 (Cell Signaling Technology 2569S). The sonicated lysates were then incubated overnight at 4° C. with the antibody-bound magnetic beads, following which the beads were sequentially washed with low-salt buffer (50 mM HEPES-KOH (pH 7.5), 0.1% SDS, 1% Triton X-100, 0.1% sodium deoxycholate, 1 mM EGTA, 1 mM EDTA, 140 mM NaCl), high-salt buffer (50 mM HEPES-KOH (pH 7.5), 0.1% SDS, 1% Triton X-100, 0.1% sodium deoxycholate, 1 mM EGTA, 1 mM EDTA, 500 mM NaCl), LiCl buffer (20 mM Tris-HCl (pH 8), 0.5% NP-40, 0.5% sodium deoxycholate, 1 mM EDTA, 250 mM LiCl), and Tris-EDTA (TE) buffer. DNA was then eluted in elution buffer (50 mM Tris-HCl (pH 8.0), 10 mM EDTA, 1% SDS) by incubation at 65° C. for 40 min accompanied by constant shaking at 1000 rpm. Magnetic beads were pelleted by high-speed centrifugation and supernatants containing the eluted fraction were reverse crosslinked overnight at 65° C. in the presence of 300 mM NaCl. The following day, RNA and protein were digested using RNase A and proteinase K, respectively, and DNA was purified with phenol-chloroform extraction and ethanol precipitation. Purified ChIP DNA was dissolved in 50 μl of 1×TE buffer. Quantitative PCR was performed on a ViiA 7 Real-Time PCR system (Thermo Fisher Scientific) with 2 μl purified DNA, 1× PowerTrack SYBR Green PCR master mix (Thermo Fisher Scientific), and PCR primers (200 nM) against the genomic regions of interest. Each individual biological sample was amplified in technical duplicate. β-Catenin and TCF4 occupancies at target gene promoters (FN1, PITX2, and COL3A1) were calculated as fold-enrichment relative to a negative control region (GAPDH promoter lacking TCF4 binding sites) using the 2^{-ΔΔCt} quantification method. First, the A Ct for each primer set was calculated using the following formula: Δ Ct=ChIP-DNA replicate average−input replicate average. Next, the final Δ Ct (ΔΔ Ct) for each target primer ChIP-DNA was calculated using the following formula: ΔΔ Ct=Δ Ct target primer set−Δ Ct negative (GAPDH) primer set. Finally, fold-enrichment values at target genes were normalized to NGP^{CRISPR ctrl} to quantify the relative changes in β-Catenin and TCF4 binding in NGP^{ALK(LF655del)} cells.

### Subcellular Fractionation

Isolation of subcellular fraction was conducted with Nuclei EZ Prep Nuclei Isolation Kit (Sigma) following the manufacturer's instructions.

### Fluorescence-Activated Cell Sorting Analysis

For cell cycle analysis, cells were trypsinized and fixed in ice-cold 70% ethanol overnight at −20° C. After washing with ice-cold phosphate-buffered saline (PBS), cells were then treated with 0.5 mg/ml RNAse A (Sigma-Aldrich) in combination with 50 μg/ml propidium iodide (PI, BD Biosciences). All FACS samples were analyzed on a FACSCanto II cell analyzer (Becton Dickinson) using Cell Quest software (Becton Dickinson). A minimum of 50,000 events were counted per sample and used for further analysis. Data were analyzed using FlowJo v10 software.

### Quantification and Statistical Analysis

The two-tailed student t-test was used to generate statistical significance for pairwise comparisons unless stated otherwise. Survival analysis was performed using the Kaplan-Meier method. The two-sided log-rank test was used to analyze the differences between two group of mice.

### Key Resource Table:

| | | |
|---|---|---|
| REAGENT or RESOURCE | SOURCE | IDENTIFIER |

Antibodies

| | | |
|---|---|---|
| Rabbit monoclonal anti-GAPDH | Cell Signaling | Cat#2118; |
| | Technology | RRID:AB_561053 |
| Mouse monoclonal anti-ALK (4C5B8) | Life Technologies | Cat#354300; |
| | | RRID:AB_2533203 |
| Rabbit monoclonal anti-ALK (C26G7) | Cell Signaling | Cat#3333; |
| | Technology | RRID:AB_836862 |
| Rabbit polyclonal anti-Phospho-ALK (Tyr1507) | Abcam | Cat# ab73996; |
| | | RRID:AB_2226754 |
| Rabbit monoclonal anti-Phospho-ALK (Tyr1604) | Cell Signaling | Cat#3341; |
| | Technology | RRID:AB_331047 |
| Rabbit monoclonal anti-E-Cadherin (24E10) | Cell Signaling | Cat#3195; |
| | Technology | RRID:AB_2291471 |
| Rabbit Polyclonal anti-ß-Actin | Cell Signaling | Cat#4967; |
| | Technology | RRID:AB_330288 |
| Rabbit monoclonal anti-p44/42 MAPK | Cell Signaling | Cat#4695; |
| | Technology | RRID:AB_390779 |
| Rabbit monoclonal anti-Phospho-p44/42 | Cell Signaling | Cat#4377; |
| MAPK(Thr202/Tyr204) | Technology | RRID:AB_331775 |
| Rabbit monoclonal anti-Stat3 | Cell Signaling | Cat#4904; |
| | Technology | RRID:AB_331269 |
| Rabbit Polyclonal anti-Phospho-Stat3(Tyr705) | Cell Signaling | Cat#9131; |
| | Technology | RRID:AB_331586 |
| Rabbit monoclonal anti-Akt | Cell Signaling | Cat#4691; |
| | Technology | RRID:AB_915783 |
| Rabbit Polyclonal anti-Phospho-Akt (Ser473) | Cell Signaling | Cat#9271; |
| | Technology | RRID:AB_329825 |
| Rabbit Polyclonal anti-Phospho-Akt (Thr308) | Cell Signaling | Cat#9275; |
| | Technology | RRID:AB_329828 |
| Mouse monoclonal anti-ALK (F-12) | Santa Cruz | sc-398791; |
| | Biotechnology | |
| Rabbit monoclonal anti-Snail | Cell Signaling | Cat# 3879; |
| | Technology | RRID:AB_2255011 |
| Rabbit monoclonal anti-Slug | Cell Signaling | Cat# 9585; |
| | Technology | RRID:AB_2239535 |
| Rabbit monoclonal anti-N-Cadherin | Cell Signaling | Cat#13116; |
| | Technology | RRID:AB_2687616 |
| Rabbit monoclonal anti-MMP-9 | Cell Signaling | Cat# 13667; |
| | Technology | RRID:AB_2798289 |
| Rabbit monoclonal anti-HA-Tag (C29F4) | Cell Signaling | Cat# 3724; |
| | Technology | RRID:AB_1549585 |
| Rabbit monoclonal anti-DYKDDDDK (SEQ ID | Cell Signaling | Cat# 14793; |
| NO: 74) Tag(D6W5B) | Technology | RRID:AB_2572291 |
| Mouse monoclonal anti-ADAM10 | Abcam | Cat# ab73402; |
| | | RRID:AB_10562808 |
| Mouse monoclonal anti-ADAM17 | Abcam | Cat# ab57484; |
| | | RRID:AB_940131 |
| Rabbit monoclonal anti-ß-Catenin (D10A8) | Cell Signaling | Cat# 8480; |
| | Technology | RRID: |
| | | AB_11127855 |
| Goat anti-Rabbit IgG (H + L) Secondary | Thermo Fisher | Cat# 65-6140 |
| Antibody, Biotin Conjugate | | RRID: |
| | | AB_2533969 |
| Alexa Fluor 568 Donkey Anti-Mouse IgG | Thermo Fisher | Cat# A10037 |
| | | RRID: |
| | | AB_2534013 |
| Mouse monoclonal anti-ALK 8G7 | This paper | N/A |

Bacterial and Virus Strains

| | | |
|---|---|---|
| DH5α. E. coli | Thermo Fisher | Cat# 18265017 |
| Stbl3 E. coli | Life Technologies | Cat# C7373-03 |

Biological Samples

| | | |
|---|---|---|
| Patient-derived xenografts (PDX) tumor tissue | St. Jude Children's | SJNBL046148_X1 |
| | Research Hospital | |
| Patient-derived xenografts (PDX) tumor tissue | St. Jude Children's | SJNBL013761_1 |
| | Research Hospital | |

Chemicals, Peptides, and Recombinant Proteins

| | | |
|---|---|---|
| Full-length recombinant human MMP-9 | Biolegend | Cat# 551102 |
| CTK8G1150 | Millipore | Cat# 444278; CAS |
| | | 1177749-58-4 |
| CAS 204140-01-2 | Millipore | Cat# 444252 |
| GM6001 | Enzo Life Sciences | Cat# 89158-002 |
| MMP408 | Millipore | Cat# 444291; CAS |
| | | 1258003-93-8 |
| Streptavidin-TXRD | SouthernBiotech | Cat# 7100-07 |
| | | RRID: |
| | | AB_2533969 |

Critical Commercial Assays

| | | |
|---|---|---|
| CellTiter-Glo Luminescent Cell Viability Assay | Promega | Cat# G7573 |
| SuperScript III Reverse Transcriptase | Thermo Fisher | Cat# 18080044 |
| QIAamp DNA mini Kit | Qiagen | Cat# 51304 |
| QCM ECMatrix Cell Invasion Assay | MilliporeSigma | Cat# ECM554 |
| 24-Well Translucent Cell Culture Inserts | Falcon | Cat# 353097 |
| FLAG ® M Purification Kit-For Mammalian | Sigma-Aldrich | Cat# CELLMM2 |
| expression systems | | |
| Lipofectamine 3000 | Life Technologies | Cat# L3000015 |
| QuikChange XL II Site-Directed Mutagenesis | Agilent | Cat# 200522 |
| Kit | Technologies | |
| Pierce Cell surface protein isolation kit | Thermo Scientific | Cat# 89881 |
| mirVana miRNA Isolation Kit | Life Technologies | Cat# AM1561 |
| QuantiFast SYBR Green PCR kit | Qiagen | Cat# 204057 |
| Universal Tyrosine Kinase Assay Kit | Takara | Cat# MK410 |
| DC protein assay kit | Bio-Rad | Cat# 500-0111 |

Deposited Data

| | | |
|---|---|---|
| Microarray data | This paper | GEO: GSE140025 |

Experimental Models: Cell Lines

| | | |
|---|---|---|
| Human: HEK293T | ATCC | CRL-3216 |
| Human: NGP | Children's Oncology | N/A |
| | Group | |
| Human: Kelly | Children's Oncology | N/A |
| | Group | |
| Human: IMR-5 | Children's Oncology | N/A |
| | Group | |
| Human: BE(2)-C | ATCC | CRL-2268 |
| Human: NBL-S | Children's Oncology | N/A |
| | Group | |
| Human: SH-SY5Y | Children's Oncology | CRL-226 |
| | Group | |
| Mouse: NIH3T3 | ATCC | CRL-1658 |
| Human: SK-N-AS | Children's Oncology | N/A |
| | Group | |
| Human: CHP-212 | ATCC | CRL-2273 |

Experimental Models: Organisms/Strains

| | | |
|---|---|---|
| Mouse: C57BL/6J | The Jackson | JAX: 000664 |
| | Laboratory | |
| Mouse: NOD/SCID | UHN (University | N/A |
| | Health Network) | |

Oligonucleotides

| | | |
|---|---|---|
| shRNA targeting sequence: MMP-9 1#: | Sigma | TRCN0000373008 |
| CATTCAGGGAGACGCCCATTT (SEQ ID NO: | | |
| 75) | | |
| shRNA targeting sequence: MMP-9 2#: | Sigma | TRCN0000051438 |
| CCACAACATCACCTATTGGAT (SEQ ID NO: | | |
| 76) | | |
| shRNA targeting sequence: ADAM10 | Sigma | TRCN0000006674 |
| 1#: CCCTACAAATCCTTTCCGTTT (SEQ ID NO: | | |
| 77) | | |
| shRNA targeting sequence: ADAM10 | Sigma | TRCN0000006673 |
| 2#: GCAGGTTCTATCTGTGAGAAA (SEQ ID | | |
| NO: 78) | | |
| shRNA targeting sequence: ADAM17 | Sigma | TRCN0000052171 |
| 1#: CCTGGTTACAACTCATGAATT (SEQ ID | | |
| NO: 79) | | |
| shRNA targeting sequence: ADAM17 | Sigma | TRCN0000052170 |
| 2#: CCCATGAAGAACACGTGTAAA (SEQ ID | | |
| NO: 80) | | |
| shRNA targeting sequence: ADAM17 | Sigma | TRCN0000052172 |
| 3#: CCTATGTCGATGCTGAACAAA (SEQ ID | | |
| NO: 81) | | |
| shRNA targeting sequence: ADAM17 | Sigma | TRCN0000052168 |
| 4#: CCAGCAGCATTCGGTAAGAAA (SEQ ID | | |
| NO: 82) | | |
| shRNA targeting sequence: ADAM17 | Sigma | TRCN0000052169 |
| 5#: GCAGCGTCAGAATCGTGTTAA (SEQ ID | | |
| NO: 83) | | |
| shRNA targeting sequence: GFP | Sigma | SHC005 |
| Primers and Oligonucleotides see Table S1. | | |

Recombinant DNA

| | | |
|---|---|---|
| pcDNA3.1-ALK-HA | This paper | N/A |
| pcDNA3.1-ALK LF655del-HA | This paper | N/A |
| pTT-FAM150A-HA | Guan et al., 2015 | N/A |
| pcDNA3-FAM150B-HA | Guan et al., 2015 | N/A |
| TGL reporter | Seong, et al., 2017 | N/A |
| pSpCas9(BB)-2A-Puro (PX459) V2 | Ran, et al., 2013 | Addgene plasmid: |
| | | Cat# 62988; |
| | | RRID:Addgene_62988 |
| PX459-ALK-gRNA | This paper | N/A |
| pLKO.1-puro eGFP shRNA Control Plasmid | Sigma | Cat# SHC005 |
| MSCV-ALK | This paper | N/A |
| MSCV-ALK LF655del | This paper | N/A |
| MSCV-ALK 655-1604 | This paper | N/A |
| MSCV-ALK Δ1-654 | This paper | N/A |
| MSCV-ALK p140 | This paper | N/A |
| MSCV-ALK Δ636-682 | This paper | N/A |
| MSCV-FLAG-ALK | This paper | N/A |
| MSCV-FLAG-ALK LF655del | This paper | N/A |
| MSCV-ALK F656D | This paper | N/A |
| MSCV-LF655RD | This paper | N/A |
| MSCV-LF655FY | This paper | N/A |
| MSCV-NLF655KRD | This paper | N/A |
| MSCV-ALK KSRNLF651del | This paper | N/A |
| MSCV-ALK KSRNLFER651del | This paper | N/A |
| MSCV-ALK ECD del | This paper | N/A |
| pcDNA3.1-ALK | This paper | N/A |
| pcDNA3.1-ALK L655K | This paper | N/A |

Software and Algorithms

| | | |
|---|---|---|
| GraphPad Prism 7.0 and 8.0 | GraphPad | https://www.graphpad. |
| | | com/scientific- |
| | | software/prism/ |
| PANTHER Classification System | PANTHER | http://pantherdb.org/ |
| | Classification | |
| | System | |
| GSEA | Broad Institute | http://software. |
| | | broadinstitute.org/ |
| | | gsea/index.jsp |
| Scaffold | Proteome Software, | http://www. |
| | Inc | proteomesoftware.com/ |
| | | products/scaffold/ |
| Image J | Schneider et al., | https://imagej.nih. |
| | 2012 | gov/ij/download.html |
| PROSPER | Monash University; | https://prosper.erc. |
| | Song et al., 2012 | monash.edu.au/home.html |
| CleavPredict | Sanford Burnham | http://cleavpredict. |
| | Medical Research | sanfordburnham.org/ |
| | Institute; | |
| | Kumar et al., 2015 | |
| FlowJo | FlowJo LLC | https://www.flowjo.com/ |

Those skilled in the art will recognize, or be able to ascertain, using no more than routine experimentation, numerous equivalents to the specific substances and procedures described herein. Such equivalents are considered to be within the scope of this invention, and are covered by the following claims.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

1. An isolated monoclonal antibody or antigen-binding fragment thereof that specifically binds to Anaplastic Lymphoma Kinase (ALK), comprising:
a. a variable heavy (VH) chain having an amino acid sequence at least 90% identical to SEQ ID NO: 03, and a variable light (VL) chain having an amino acid sequence at least 90% identical to SEQ ID NO: 04;
b. a variable heavy (VH) chain having an amino acid sequence at least 90% identical to SEQ ID NO: 13, and a variable light (VL) chain having an amino acid sequence at least 90% identical to SEQ ID NO: 14; or
c. a variable heavy (VH) chain having an amino acid sequence at least 90% identical to SEQ ID NO: 23, and a variable light (VL) chain having an amino acid sequence at least 90% identical to SEQ ID NO: 24.

2. The monoclonal antibody or fragment of claim 1, wherein the ALK is human ALK.

3. (canceled)

4. The monoclonal antibody or fragment of claim 1, wherein the monoclonal antibody or fragment is:
a. fully human or humanized;
b. monospecific, bispecific, or multispecific,
c. an IgG or a single chain (sc) fragment.

5-7. (canceled)

8. The monoclonal antibody or fragment of claim 1, wherein the monoclonal antibody or fragment competes for binding of ALK with a monoclonal antibody comprising:
a. a variable heavy (VH) chain having an amino acid sequence identical to SEQ ID NO: 03, and a variable light (VL) chain having an amino acid sequence identical to SEQ ID NO: 04;
b. a variable heavy (VH) chain having an amino acid sequence identical to SEQ ID NO: 13, and a variable light (VL) chain having an amino acid sequence identical to SEQ ID NO: 14; or
c. a variable heavy (VH) chain having an amino acid sequence identical to SEQ ID NO: 23, and a variable light (VL) chain having an amino acid sequence identical to SEQ ID NO: 24.

9. (canceled)

10. The monoclonal antibody or fragment of claim 1, comprising:
a. a heavy chain with three CDRs comprising the amino acid sequences GYTFTDYEMH (SEQ ID NO:5), AIDPETGGSAYNQKFKA (SEQ ID NO:6), and TRYYGSSPAMDY (SEQ ID NO:7) respectively, and/or a light chain with three CDRs comprising the amino acid sequences SASQGIGNYLN (SEQ ID NO:8), YTSSLHS (SEQ ID NO:9), and QQYSKLPYT (SEQ ID NO:10) respectively;
b. a heavy chain with three CDRs comprising the amino acid sequences GYTFTDYE (SEQ ID NO:15), IDPETGGS (SEQ ID NO:16), and TRTRYYGSSPAMDY (SEQ ID NO:17) respectively, and/or a light chain with three CDRs comprising the amino acid sequences QGIGNY (SEQ ID NO:18), YTS (SEQ ID NO:19), and QQYSKLPYT (SEQ ID NO:20) respectively; and
c. heavy chain with three CDRs comprising the amino acid sequences GYTFTDYE (SEQ ID NO:25), IDPETGGT (SEQ ID NO:26), and TRCYYGSSWYFDV (SEQ ID NO:27) respectively, and/or a light chain with three CDRs comprising the amino acid sequences QGITNY (SEQ ID NO:28), YTS (SEQ ID NO:29), and QQYSKVPRT (SEQ ID NO:30) respectively.

11-12. (canceled)

13. The monoclonal antibody or fragment of claim 1, comprising:
a. a variable heavy (VH) chain having an amino acid sequence identical to SEQ ID NO: 03, and a variable light (VL) chain having an amino acid sequence identical to SEQ ID NO: 04;
b. a variable heavy (VH) chain having an amino acid sequence SEQ ID NO: 13, and a variable light (VL) chain having an amino acid sequence identical to SEQ ID NO: 14; or
c. a variable heavy (VH) chain having an amino acid sequence SEQ ID NO: 23, and a variable light (VL) chain having an amino acid sequence identical to SEQ ID NO: 24.

14-16. (canceled)

17. The monoclonal antibody or fragment of claim 1, wherein the monoclonal antibody or fragment is linked to a therapeutic agent, optionally wherein the therapeutic agent is a toxin, a radiolabel, a siRNA, a small molecule, or a cytokine.

18-19. (canceled)

20. A nucleic acid encoding the monoclonal antibody or fragment of claim 1, wherein the ALK is human ALK.

21. A vector comprising the nucleic acid of claim 20, the nucleic acid having:
a. a portion having a sequence at least 90% identical to SEQ ID NO: 01, and/or a portion having a sequence at least 90% identical to SEQ ID NO: 02;
b. a portion having a sequence at least 90% identical to SEQ ID NO: 11, and/or a portion having a sequence at least 90% identical to SEQ ID NO: 12; or
C. a portion having a sequence at least 90% identical to SEQ ID NO: 21, and/or a portion having a sequence at least 90% identical to SEQ ID NO: 22.

22. A cell comprising the vector of claim 21, wherein the vector is a viral vector.

23. (canceled)

24. A kit comprising:
the monoclonal antibody or fragment of claim 1, wherein the ALK is human ALK;
a syringe, needle, or applicator for administration of the monoclonal antibody or fragment to a subject; and
instructions for use.

25-40. (canceled)

41. A pharmaceutical composition comprising a monoclonal antibody or antigen-binding fragment thereof that specifically binds to Anaplastic Lymphoma Kinase (ALK), and a pharmaceutically acceptable carrier or excipient, wherein the monoclonal antibody or fragment comprises:
a. a variable heavy (VH) chain having an amino acid sequence at least 90% identical to SEQ ID NO: 03, and a variable light (VL) chain having an amino acid sequence at least 90% identical to SEQ ID NO: 04;
b. a variable heavy (VH) chain having an amino acid sequence at least 90% identical to SEQ ID NO: 13, and a variable light (VL) chain having an amino acid sequence at least 90% identical to SEQ ID NO: 14; or
c. a variable heavy (VH) chain having an amino acid sequence at least 90% identical to SEQ ID NO: 23, and a variable light (VL) chain having an amino acid sequence at least 90% identical to SEQ ID NO: 24.

42-43. (canceled)

44. An engineered cell comprising a chimeric antigen receptor, wherein:
the chimeric antigen receptor comprises an extracellular ligand binding domain that is specific for an antigen on the surface of a cancer cell, wherein the antigen comprises anaplastic lymphoma kinase (ALK);
the extracellular ligand binding domain comprises a monoclonal antibody or antigen binding fragment thereof; and
the monoclonal antibody or antigen binding fragment thereof comprises
a. a variable heavy (VH) chain having an amino acid sequence at least 90% identical to SEQ ID NO: 03, and a variable light (VL) chain having an amino acid sequence at least 90% identical to SEQ ID NO: 04;
b. a variable heavy (VH) chain having an amino acid sequence at least 90% identical to SEQ ID NO: 13, and a variable light (VL) chain having an amino acid sequence at least 90% identical to SEQ ID NO: 14; or
c. a variable heavy (VH) chain having an amino acid sequence at least 90% identical to SEQ ID NO: 23, and a variable light (VL) chain having an amino acid sequence at least 90% identical to SEQ ID NO: 24.

45-50. (canceled)

51. The engineered cell of claim 44, wherein the engineered cell is a T cell, an NK cell, or an NKT cell, optionally, wherein the T cell is CD4+, CD8+, CD3+ pan T cells, or any combination thereof.

52. (canceled)

53. A kit comprising:
the engineered cell of claim 44, wherein the engineered cell is a T cell, an NK cell, or an NKT cell;
a syringe, needle, or applicator for administration of the engineered cell to a subject; and
instructions for use.

54. A pharmaceutical composition comprising the engineered cell of claim 44, wherein the engineered cell is a T cell, an NK cell, or an NKT cell, and a pharmaceutically acceptable carrier or excipient.

55-56. (canceled)

57. A method of detecting the presence of a peripheral sympathetic nervous system cancer in a sample, the method comprising contacting the sample with the monoclonal antibody of claim 1, wherein the ALK is human ALK; and detecting the presence or absence of an antibody-antigen complex, thereby detecting the presence of ALK in the sample.

58-86. (canceled)

87. A method of treating cancer in a subject, the method comprising administering to a subject in need thereof a therapeutically effective amount of a composition comprising the engineered cell comprising a CAR of claim 44, wherein the engineered cell is a T cell, an NK cell, or an NKT cell.

88-91. (canceled)

92. A method of decreasing metastasis in a subject, the method comprising administering to a subject in need thereof a therapeutically effective amount of a composition comprising the engineered cell comprising a CAR of claim 44, wherein the engineered cell is a T cell, an NK cell, or an NKT cell.

93. The method of claim 92, wherein the subject is afflicted with a cancer that expresses ALK, optionally, wherein the ALK comprises full-length ALK and wherein the cancer comprises neuroblastoma, glioma, rhabdomyosarcoma, non-small cell lung cancer, inflammatory myofibroblastic tumors, anaplastic large cell lymphoma, or thyroid cancer.

94-96. (canceled)
